(19) United States
(12) Patent Application Publication
(10) Pub. No.: US 2014/0135482 A1
(43) Pub. Date: May 15, 2014

(54) Bispecific Anti ErbB3 / Anti cMet Antibodies

(71) Applicant: ROCHE GLYCART AG, SCHLIEREN (CH)

(72) Inventors: Birgit Bossenmaier, Seefeld (DE); Ulrich Brinkmann, Weilheim (DE); Wilma Dormeyer, Muenchen (DE); Eike Hoffmann, Seefeld (DE); Christian Klein, Bonstetten (CH); Gerhard Niederfellner, Oberhausen (DE); Juergen Michael Schanzer, Traunstein (DE); Jan Olaf Stracke, Muenchen (DE); Claudio Sustmann, Muenchen (DE); Pablo Umana, Zuerich (CH)

(73) Assignee: ROCHE GLYCART AG, SCHLIEREN (CH)

(21) Appl. No.: 14/157,332

(22) Filed: Jan. 16, 2014

Related U.S. Application Data
(63) Continuation of application No. 13/788,435, filed on Mar. 7, 2013, now abandoned.
Application No. 13/788,435 is a continuation of application No. 12/752,196, filed on Apr. 1, 2010, now abandoned.

(30) Foreign Application Priority Data
Apr. 7, 2009 (EP) 09005110.3

(51) Int. Cl. C07K 16/46

(52) U.S. Cl. CPC C07K 16/468
USPC 530/387.3; 536/23.4

(57) ABSTRACT

The present invention relates to bispecific antibodies against human ErbB-3 and against human c-Met, methods for their production, pharmaceutical compositions containing the antibodies, and uses thereof.

### PRIORITY TO RELATED APPLICATION(S)

This application is a continuation of U.S. patent application Ser. No. 13/788,435, filed Mar. 7, 2013, which is a continuation of U.S. patent application Ser. No. 12/752,196, filed Apr. 1, 2010, which claims the benefit of European Patent Application No. 09005110.3, filed Apr. 7, 2009, which are hereby incorporated by reference in their entirety.

### SEQUENCE LISTING

The instant application contains a Sequence Listing which has been submitted via EFS-Web and is hereby incorporated by reference in its entirety. Said ASCII copy, created on Jan. 15, 2014, is named 26064.txt and is 195,144 bytes in size.

### FIELD OF THE INVENTION

The present invention relates to bispecific antibodies against human ErbB-3 and against human c-Met, methods for their production, pharmaceutical compositions containing the antibodies, and uses thereof.

### BACKGROUND OF THE INVENTION

### ErbB Protein Family

The ErbB protein family consists of 4 members: ErbB-1, also named epidermal growth factor receptor (EGFR), ErbB-2, also named HER2 in humans and neu in rodents, ErbB-3, also named HER3 and ErbB-4, also named HER4.

### ErbB-3 and Anti-ErbB-3 Antibodies

ErbB-3 (also known as V-erb-b2 erythroblastic leukemia viral oncogene homolog 3 (avian), ERBB3, HER3; SEQ ID NO:46) is membrane-bound protein which has a neuregulin binding domain but not an active kinase domain (Kraus, M. H, et al., Proc. Natl. Acad. Sci. U.S.A. 86 (1989) 9193-7; Plowman, G. D., et al., Proc. Natl. Acad. Sci. U.S.A. 87 (1999) 4905-9; Katoh, M., et al., Biochem. Biophys. Res. Commun. 192 (1993) 1189-97). It therefore can bind this ligand but not convey the signal into the cell through protein phosphorylation. However, it does form heterodimers with other EGF receptor family members which do have kinase activity. Heterodimerization leads to the activation of pathways which lead to cell proliferation or differentiation. Amplification of this gene and/or overexpression of its protein have been reported in numerous cancers, including prostate, bladder, and breast tumors. Alternate transcriptional splice variants encoding different isoforms have been characterized. One isoform lacks the intermembrane region and is secreted outside the cell. This form acts to modulate the activity of the membrane-bound form (Corfas, G., et al., 7(6) (2004) 575-80). It is thought that ERBB3, when activated, becomes a substrate for dimerization and subsequent phosphorylation by ERBB1, ERBB2 and ERBB4. Like many of the receptor tyrosine-kinases, ERBB3 is activated by extracellular ligand. Ligands known to bind to ERBB3 include heregulin.

Anti-ErbB-3 antibodies for use in anti-cancer therapy are known e.g. from WO 97/35885, WO 2007/077028 or WO 2008/100624.

c-Met and Anti-c-Met Antibodies

MET (mesenchymal-epithelial transition factor) is a proto-oncogene that encodes a protein MET, (also known as c-Met; hepatocyte growth factor receptor HGFR; HGF receptor; scatter factor receptor; SF receptor; SEQ. ID. NO:45) (Dean, M., et al., Nature 318 (1985) 385-8 (1985); Chan, A. M., et al., Oncogene 1 (1987) 229-33; Bottaro, D. P., et al., Science 251 (1991) 802-4; Naldini, L., et al., EMBO J. 10 (1991) 2867-78; Maulik, G., et al, Cytokine Growth Factor Rev. 13 (2002) 41-59). MET is a membrane receptor that is essential for embryonic development and wound healing. Hepatocyte growth factor (HGF) is the only known ligand of the MET receptor. MET is normally expressed by cells of epithelial origin, while expression of HGF is restricted to cells of mesenchymal origin. Upon HGF stimulation, MET induces several biological responses that collectively give rise to a program known as invasive growth. Abnormal MET activation in cancer correlates with poor prognosis, where aberrantly active MET triggers tumor growth, formation of new blood vessels (angiogenesis) that supply the tumor with nutrients, and cancer spread to other organs (metastasis). MET is deregulated in many types of human malignancies, including cancers of kidney, liver, stomach, breast, and brain. Normally, only stem cells and progenitor cells express MET, which allows these cells to grow invasively in order to generate new tissues in an embryo or regenerate damaged tissues in an adult. However, cancer stem cells are thought to hijack the ability of normal stem cells to express MET, and thus become the cause of cancer persistence and spread to other sites in the body.

The proto-oncogene MET product is the hepatocyte growth factor receptor and encodes tyrosine-kinase activity. The primary single chain precursor protein is post-translationally cleaved to produce the alpha and beta subunits, which are disulfide linked to form the mature receptor. Various mutations in the MET gene are associated with papillary renal carcinoma.

Anti-c-Met antibodies are known e.g. from U.S. Pat. No. 5,686,292, U.S. Pat. No. 7,476,724, WO 2004072117, WO 2004108766, WO 2005016382, WO 2005063816, WO 2006015371, WO 2006104911, WO 2007126799, or WO 2009007427.

C-Met binding peptides are known e.g. from Matzke, A., et al, Cancer Res 2005; 65: (14) and Tam, E. M, et al., J. Mol. Biol. 385 (2009) 79-90.

### Bispecific Antibodies

A wide variety of recombinant antibody formats have been developed in the recent past, e.g. tetravalent bispecific antibodies by fusion of, e.g., an IgG antibody format and single chain domains (see e.g. Coloma, M. J., et al., Nature Biotech 15 (1997) 159-163; WO 2001/077342; and Morrison, S. L., Nature Biotech 25 (2007) 1233-1234).

Also several other new formats wherein the antibody core structure (IgA, IgD, IgE, IgG or IgM) is no longer retained such as dia-, tria- or tetrabodies, minibodies, several single chain formats (scFv, Bis-scFv), which are capable of binding two or more antigens, have been developed (Holliger, P, et al., Nature Biotech 23 (2005) 1126-1136; Fischer, N., Léger, O., Pathobiology 74 (2007) 3-14; Shen, J, et al., Journal of Immunological Methods 318 (2007) 65-74; Wu, C., et al., Nature Biotech. 25 (2007) 1290-1297).

All such formats use linkers either to fuse the antibody core (IgA, IgD, IgE, IgG or IgM) to a further binding protein (e.g. scFv) or to fuse e.g. two Fab fragments or scFvs (Fischer, N., Léger, O., Pathobiology 74 (2007) 3-14). It has to be kept in mind that one may want to retain effector functions, such as e.g. complement-dependent cytotoxicity (CDC) or antibody dependent cellular cytotoxicity (ADCC), which are mediated through the Fc receptor binding, by maintaining a high degree of similarity to naturally occurring antibodies.

In WO 2007/024715 are reported dual variable domain immunoglobulins as engineered multivalent and multispecific binding proteins. A process for the preparation of biologically active antibody dimers is reported in U.S. Pat. No. 6,897,044. Multivalent FV antibody construct having at least four variable domains which are linked with each over via peptide linkers are reported in U.S. Pat. No. 7,129,330. Dimeric and multimeric antigen binding structures are reported in US 2005/0079170. Tri- or tetra-valent monospecific antigen-binding protein comprising three or four Fab fragments bound to each other covalently by a connecting structure, which protein is not a natural immunoglobulin are reported in U.S. Pat. No. 6,511,663. In WO 2006/020258 tetravalent bispecific antibodies are reported that can be efficiently expressed in prokaryotic and eukaryotic cells, and are useful in therapeutic and diagnostic methods. A method of separating or preferentially synthesizing dimers which are linked via at least one interchain disulfide linkage from dimers which are not linked via at least one interchain disulfide linkage from a mixture comprising the two types of polypeptide dimers is reported in US 2005/0163782. Bispecific tetravalent receptors are reported in U.S. Pat. No. 5,959,083. Engineered antibodies with three or more functional antigen binding sites are reported in WO 2001/077342.

Multispecific and multivalent antigen-binding polypeptides are reported in WO 1997/001580. WO 1992/004053 reports homoconjugates, typically prepared from monoclonal antibodies of the IgG class which bind to the same antigenic determinant are covalently linked by synthetic cross-linking Oligomeric monoclonal antibodies with high avidity for antigen are reported in WO 1991/06305 whereby the oligomers, typically of the IgG class, are secreted having two or more immunoglobulin monomers associated together to form tetravalent or hexavalent IgG molecules. Sheep-derived antibodies and engineered antibody constructs are reported in U.S. Pat. No. 6,350,860, which can be used to treat diseases wherein interferon gamma activity is pathogenic. In US 2005/0100543 are reported targetable constructs that are multivalent carriers of bi-specific antibodies, i.e., each molecule of a targetable construct can serve as a carrier of two or more bi-specific antibodies. Genetically engineered bispecific tetravalent antibodies are reported in WO 1995/009917. In WO 2007/109254 stabilized binding molecules that consist of or comprise a stabilized scFv are reported. US 2007/0274985 relates to antibody formats comprising single chain Fab (scFab) fragments.

WO2009111707(A1) relates to a combination therapy with Met and HER antagonists. WO2009111691(A2A3) to a combination therapy with Met and EGFR antagonists.

WO 2008/100624 relates to anti-ErbB-3 antibodies with increased ErbB-3 receptor internalization and their use in bispecific antibodies inter alia with c-Met as second antigen.

### SUMMARY OF THE INVENTION

A first aspect of the current invention is a bispecific antibody specifically binding to human ErbB-3 and human c-Met comprising a first antigen-binding site that specifically binds to human ErbB-3 and a second antigen-binding site that specifically binds to human c-Met, characterized in that the bispecific antibody shows an internalization of ErbB-3 of no more than 15% when measured after 2 hours in a flow cytometry assay on A431 cells, as compared to internalization of ErbB-3 in the absence of antibody.

In one embodiment of the invention the antibody is a bivalent or trivalent, bispecific antibody specifically binding to human ErbB-3 and human c-Met comprising one or two antigen-binding sites that specifically bind to human ErbB-3 and one antigen-binding site that specifically binds to human c-Met.

In one embodiment of the invention the antibody is a bivalent, bispecific antibody specifically binding to human ErbB-3 and human c-Met comprising one antigen-binding site that specifically binds to human ErbB-3 and one antigen-binding site that specifically binds to human c-Met.

In one preferred embodiment of the invention the antibody is a trivalent, bispecific antibody specifically binding to human ErbB-3 and human c-Met comprising two antigen-binding sites that specifically bind to human ErbB-3 and a third antigen-binding site that specifically binds to human c-Met.

One aspect of the invention is a bispecific antibody specifically binding to human ErbB-3 and human c-Met comprising a first antigen-binding site that specifically binds to human ErbB-3 and a second antigen-binding site that specifically binds to human c-Met, characterized in that
i) the first antigen-binding site comprises in the heavy chain variable domain a CDR3H region of SEQ ID NO: 53, a CDR2H region of SEQ ID NO: 54, and a CDR1H region of SEQ ID NO:55, and in the light chain variable domain a CDR3L region of SEQ ID NO: 56, a CDR2L region of SEQ ID NO:57, and a CDR1L region of SEQ ID NO:58 or a CDR1L region of SEQ ID NO:59; and
   the second antigen-binding site comprises in the heavy chain variable domain a CDR3H region of SEQ ID NO: 66, a CDR2H region of, SEQ ID NO: 67, and a CDR1H region of SEQ ID NO: 68, and in the light chain variable domain a CDR3L region of SEQ ID NO: 69, a CDR2L region of SEQ ID NO: 70, and a CDR1L region of SEQ ID NO: 71.
ii) the first antigen-binding site comprises in the heavy chain variable domain a CDR3H region of SEQ ID NO: 60, a CDR2H region of SEQ ID NO: 61, and a CDR1H region of SEQ ID NO:62, and in the light chain variable domain a CDR3L region of SEQ ID NO: 63, a CDR2L region of SEQ ID NO:64, and a CDR1L region of SEQ ID NO:65 or a CDR1L region of SEQ ID NO:66; and
   the second antigen-binding site comprises in the heavy chain variable domain a CDR3H region of SEQ ID NO: 66, a CDR2H region of, SEQ ID NO: 67, and a CDR1H region of SEQ ID NO: 68, and in the light chain variable domain a CDR3L region of SEQ ID NO: 69, a CDR2L region of SEQ ID NO: 70, and a CDR1L region of SEQ ID NO: 71.

The bispecific antibody is preferably, characterized in that
i) the first antigen-binding site comprises as heavy chain variable domain SEQ ID NO: 47, and as light chain variable domain SEQ ID NO: 48, and the second antigen-binding site comprises as heavy chain variable domain SEQ ID NO: 3, and as light chain variable domain a SEQ ID NO: 4;
ii) the first antigen-binding site comprises as heavy chain variable domain SEQ ID NO: 49, and as light chain variable domain SEQ ID NO: 50, and the second antigen-binding site comprises as heavy chain variable domain SEQ ID NO: 3, and as light chain variable domain a SEQ ID NO: 4;
iii) the first antigen-binding site comprises as heavy chain variable domain SEQ ID NO: 49, and as light chain variable domain SEQ ID NO: 51, and the second antigen-binding site comprises as heavy chain variable domain SEQ ID NO: 3, and as light chain variable domain a SEQ ID NO: 4;
iv) the first antigen-binding site comprises as heavy chain variable domain SEQ ID NO: 49, and as light chain variable domain SEQ ID NO: 52, and the second antigen-binding site comprises as heavy chain variable domain SEQ ID NO: 3, and as light chain variable domain a SEQ ID NO: 4; or
v) the first antigen-binding site comprises as heavy chain variable domain SEQ ID NO: 1, and as light chain variable domain SEQ ID NO: 2, and the second antigen-binding site comprises as heavy chain variable domain SEQ ID NO: 3, and as light chain variable domain a SEQ ID NO: 4; or

Preferably the bispecific antibody is characterized in that

the first antigen-binding site comprises as heavy chain variable domain SEQ ID NO: 49, and as light chain variable domain SEQ ID NO: 51, and the second antigen-binding site comprises as heavy chain variable domain SEQ ID NO: 3, and as light chain variable domain a SEQ ID NO: 4.

In one embodiment the bispecific antibody according to the invention is characterized in comprising a constant region of IgG1 or IgG3 subclass.

In one embodiment the bispecific antibody according to the invention is characterized in that the antibody is glycosylated with a sugar chain at Asn297 whereby the amount of fucose within the sugar chain is 65% or lower.

A further aspect of the invention is a nucleic acid molecule encoding a chain of the bispecific antibody.

Still further aspects of the invention are a pharmaceutical composition comprising the bispecific antibody according to the invention, the composition for the treatment of cancer, the use of the bispecific antibody for the manufacture of a medicament for the treatment of cancer, a method of treatment of patient suffering from cancer by administering the bispecific antibody to a patient in the need of such treatment.

The antibodies according to the invention show highly valuable properties like growth inhibition of cancer cells expressing both receptors <ErbB3> and <c-Met>, antitumor efficacy causing a benefit for a patient suffering from cancer. The bispecific <ErbB3-c-Met> antibodies according to the invention show reduced internalization of ErbB3/antibody complex when compared to their parent monospecific <ErbB3> antibodies on cancer cells expressing both receptors <ErbB3> and <c-Met>.

### BRIEF DESCRIPTION OF THE SEVERAL VIEWS OF THE DRAWINGS

FIG. 1 Schematic structure of a full length antibody without CH4 domain specifically binding to a first antigen 1 with two pairs of heavy and light chain which comprise variable and constant domains in a typical order.

FIG. 2a Schematic structure of a bivalent, bispecific <ErbB3-c-Met> antibody, comprising: the light chain and heavy chain of a full length antibody specifically binding to human ErbB-3; and the light chain and heavy chain of a full length antibody specifically binding to human c-Met, wherein the constant domains CL and CH1, and/or the variable domains VL and VH are replaced by each other, which are modified with knobs-into hole technology

FIG. 2b Schematic structure of a bivalent, bispecific <ErbB3-c-Met> antibody, comprising: the light chain and heavy chain of a full length antibody specifically binding to human ErbB-3; and the light chain and heavy chain of a full length antibody specifically binding to human c-Met, wherein the constant domains CL and CH1, and/or the variable domains VL and VH are replaced by each other, which are modified with knobs-into hole technology

FIG. 2c Schematic structure of a bivalent, bispecific <ErbB3-c-Met> antibody, comprising: the light chain and heavy chain of a full length antibody specifically binding to human ErbB-3; and the light chain and heavy chain of a full length antibody specifically binding to human c-Met, wherein the constant domains CL and CH1, and/or the variable domains VL and VH are replaced by each other, which are modified with knobs-into hole technology

FIG. 3a Schematic representation of a trivalent, bispecific <ErbB3-c-Met> antibody according to the invention, comprising a full length antibody specifically binding to a first antigen 1 to which two polypeptides VH and VL are fused (the VH and VL domains of both together forming a antigen binding site specifically binding to a second antigen 2;

FIG. 3b Schematic representation of a trivalent, bispecific <ErbB3-c-Met> antibody according to the invention, comprising a full length antibody specifically binding to a first antigen 1 to which two polypeptides VH-CH1 and VL-CL are fused (the VH and VL domains of both together forming a antigen binding site specifically binding to a second antigen 2)

FIG. 3c Schematic representation of a trivalent, bispecific antibody according to the invention, comprising a full length antibody specifically binding to a first antigen 1 to which two polypeptides VH and VL are fused (the VH and VL domains of both together forming a antigen binding site specifically binding to a second antigen 2) with “knobs and holes”.

FIG. 3d Schematic representation of a trivalent, bispecific antibody according to the invention, comprising a full length antibody specifically binding to a first antigen 1 to which two polypeptides VH and VL are fused (the VH and VL domains of both together forming a antigen binding site specifically binding to a second antigen 2, wherein these VH and VL domains comprise an interchain disulfide bridge between positions VH44 and VL100) with “knobs and holes”.

FIG. 4a Schematic structure of the four possible single chain Fab fragments

FIG. 4b Schematic structure of the two single chain Fv fragments

FIG. 5a Schematic structure of a trivalent, bispecific <ErbB3-c-Met> antibody comprising a full length antibody and one single chain Fab fragment

FIG. 5b Schematic structure of a trivalent, bispecific <ErbB3-c-Met> antibody comprising one single chain Fv fragment—bispecific trivalent example with knobs and holes

FIG. 6a Schematic structure of a tetravalent, bispecific <ErbB3-c-Met> antibody comprising a full length antibody and two single chain Fab fragments

FIG. 6b Schematic structure of a tetravalent, bispecific <ErbB3-c-Met> antibody comprising two single chain Fv fragments—the c-Met binding sites are derived from c-Met dimerisation inhibiting antibodies

FIG. 7 Schematic structure of a bivalent, bispecific <ErbB3-c-Met> antibody in which one Fab arm is replaced with a scFab fragment.

FIG. 8 Binding of bispecific antibodies to the cell surface of cancer cells

FIG. 9a Inhibition of HGF-induced c-Met receptor phosphorylation by bispecific Her3/c-Met antibody formats

FIG. 9b Inhibition of HGF-induced c-Met receptor phosphorylation by bispecific Her3/c-Met antibody formats

FIG. 9c Inhibition of HGF-induced c-Met receptor phosphorylation by bispecific Her3/c-Met antibody formats

FIG. 10a Inhibition of HRG-induced Her3 receptor phosphorylation by bispecific Her3/c-Met antibody formats.

FIG. 10b Inhibition of HRG-induced Her3 receptor phosphorylation by bispecific Her3/c-Met antibody formats.

FIG. 11 Inhibition of HGF-induced HUVEC proliferation by bispecific Her3/c-Met antibody formats

FIG. 12 Inhibition of HGF-induced HUVEC proliferation by bispecific Her3/c-Met antibody formats

FIG. 13 Inhibition of HGF-induced HUVEC proliferation by bispecific Her3/c-Met antibody formats

FIG. 14 Inhibition of proliferation in the cancer cell line A431 by bispecific Her3/c-Met antibody formats.

FIG. 15 Analysis of inhibition of HGF-induced cell-cell dissemination (scattering) in the cancer cell line A431 by bispecific Her3/c-Met antibody formats.

FIG. 16 Analysis of inhibition of HGF-induced cell-cell dissemination (scattering) in the cancer cell line A431 by bispecific Her3/c-Met antibody formats.

FIG. 17 Analysis of Her3 and c-Met cell surface expression in four different cancer cell lines.

FIG. 18 Analysis of antibody-mediated receptor internalization in the cancer cell lines A431, A549, and DU145.

FIG. 19 Analysis of HGF-induced cellular migration of A431 cells. A. Migration of A431 cancer cells was measured as a function of impedance in the presence of an increasing dose of the bispecific antibody MH_TvAb18. Displayed is the endpoint readout after 24 h. B. As control an unspecific human IgG control was added in a similar concentration range as the bispecific antibody.

FIG. 20a Analysis of cell-cell crosslinking by the bispecific Her3/c-Met_scFv_SSKH antibody in HT29 cells (Staining with PKH26 & PKH67 (SIGMA))

FIG. 20b Analysis of cell-cell crosslinking by the bispecific Her3/c-Met_scFv_SSKH antibody in HT29 cells (Staining with PKH26 & PKH67 (SIGMA))

FIG. 20c Analysis of cell-cell crosslinking by the bispecific Her3/c-Met_scFv_SSKH antibody in HT29 cells (Staining with PKH26 & PKH67 (SIGMA))

FIG. 20d Analysis of cell-cell crosslinking by the bispecific Her3/c-Met_scFv_SSKH antibody in HT29 cells (Staining with PKH26 & PKH67 (SIGMA))

FIG. 21 SDS page of bispecific Her3/c-Met antibodies Her3/Met_scFvSS_KH (left side) and Her3/Met_scFv_KH (right side)

FIG. 22a HP SEC Analysis (Purified Protein) of bispecific Her3/c-Met antibodies Her3/Met_scFvSSKH

FIG. 22b HP SEC Analysis (Purified Protein) of bispecific Her3/MetscFv_KH

### DETAILED DESCRIPTION OF THE INVENTION

A first aspect of the current invention is a bispecific antibody specifically binding to human ErbB-3 and human c-Met comprising a first antigen-binding site that specifically binds to human ErbB-3 and a second antigen-binding site that specifically binds to human c-Met, characterized in that the bispecific antibody shows an internalization of ErbB-3 of no more than 15% when measured after 2 hours in a flow cytometry assay on A431 cells, as compared to internalization of ErbB-3 in the absence of the bispecific antibody.

In one embodiment the a bispecific antibody specifically binding to human ErbB-3 and human c-Met comprising a first antigen-binding site that specifically binds to human ErbB-3 and a second antigen-binding site that specifically binds to human c-Met is characterized in that the bispecific antibody shows an internalization of ErbB-3 of no more than 10% when measured after 2 hours in a flow cytometry assay on A431 cells, as compared to internalization of ErbB-3 in the absence of the bispecific antibody.

In one embodiment the a bispecific antibody specifically binding to human ErbB-3 and human c-Met comprising a first antigen-binding site that specifically binds to human ErbB-3 and a second antigen-binding site that specifically binds to human c-Met is characterized in that the bispecific antibody shows an internalization of ErbB-3 of no more than 7% when measured after 2 hours in a flow cytometry assay on A431 cells, as compared to internalization of ErbB-3 in the absence of the bispecific antibody.

In one embodiment the a bispecific antibody specifically binding to human ErbB-3 and human c-Met comprising a first antigen-binding site that specifically binds to human ErbB-3 and a second antigen-binding site that specifically binds to human c-Met is characterized in that the bispecific antibody shows an internalization of ErbB-3 of no more than 5% when measured after 2 hours in a flow cytometry assay on A431 cells, as compared to internalization of ErbB-3 in the absence of the bispecific antibody.

The term “the internalization of ErbB-3” refers to the antibody-induced ErbB-3 receptor internalization on A431 cells (ATCC No. CRL-1555). as compared to the internalization of ErbB-3 in the absence of antibody. Such internalization of the ErbB-3 receptor is induced by the bispecific antibodies according to the invention and is measured after 2 hours in a flow cytometry assay (FACS) as described in Example 8. A bispecific antibody according the invention shows an internalization of ErbB-3 of no more than 15% on A431 cells after 2 hours of antibody exposure as compared to the internalization of ErbB-3 in the absence of antibody. In one embodiment the antibody shows an internalization of ErbB-3 of no more than 10%. In one embodiment the antibody shows an internalization of ErbB-3 of no more than 7%. In one embodiment the antibody shows an internalization of ErbB-3 of no more than 5% To determine whether a bispecific ErbB3/c-Met antibody shows an internalization of ErbB-3 of 10% or less after 2 hours on A431 cells it can be compared in a flow cytometry assay (FACS) with the bispecific ErbB3/c-Met antibody MH_TvAb24 described below. To determine whether a bispecific ErbB3/c-Met antibody shows an internalization of ErbB-3 of 5% or less after 2 hours on A431 cells it can be compared in a flow cytometry assay (FACS) with the bispecific ErbB3/c-Met antibody MH_TvAb29 described below.

Another aspect of the current invention is a bispecific antibody specifically binding to human ErbB-3 and human c-Met comprising a first antigen-binding site that specifically binds to human ErbB-3 and a second antigen-binding site that specifically binds to human c-Met, characterized in that the bispecific antibody reduces the internalization of ErbB-3, compared to the internalization of ErbB-3 induced by the (corresponding) monospecific parent ErbB-3 antibody, 50% or more (in one embodiment 60% or more; in another embodiment 70% or more, in one embodiment 80% or more), when measured after 2 hours in a flow cytometry assay on A431 cells (ATCC No. CRL-1555). The reduction of internalization of ErbB-3 is calculated (using the values measured after 2 hours in a flow cytometry assay on A431 cells) as follows: 100×(% internalization of ErbB induced by monospecific parent ErbB-3 antibody−% internalization of ErbB induced by bispecific ErbB-3/c-Met antibody)/% internalization of ErbB induced by monospecific parent ErbB-3 antibody. For example: a) the bispecific ErbB-3/c-Met antibody MH_TvAb21 shows an internalization of ErbB-3 of 1%, and the monospecific parent ErbB-3 antibody Mab 205 shows an internalization of ErbB-3 of 40%. Thus the bispecific ErbB-3/c-Met antibody MH_TvAb21 shows a reduction of the internalization of ErbB-3 of 100×(40−1)/40%=97.5%; b) the bispecific ErbB-3/c-Met antibody MH_TvAb25 shows an internalization of ErbB-3 of 11%, and the monospecific parent ErbB-3 antibody Mab 205 shows an internalization of ErbB-3 of 40%. Thus the bispecific ErbB-3/c-Met antibody MH_TvAb21 shows a reduction of the internalization of ErbB-3 of 100×(40−11)/40%=72.5%;.or c) the bispecific ErbB-3/c-Met antibody HER3/Met_C6 shows an internalization of ErbB-3 of 11%, and the monospecific parent ErbB-3 antibody HER3clone 29 shows an internalization of ErbB-3 of 54%. Thus the bispecific ErbB-3/c-Met antibody HER3/Met_C6 shows a reduction of the internalization of ErbB-3 of 100×(54−6)/40%=88.9%. (see internalization values measured after 2 hours in a flow cytometry assay on A431 cells in Example 8).

In one embodiment of the invention the antibody is a trivalent, bispecific antibody specifically binding to human ErbB-3 and human c-Met comprising two antigen-binding sites that specifically bind to human ErbB-3 and a third antigen-binding site that specifically binds to human c-Met.

In one embodiment of the invention the antibody is a bivalent, bispecific antibody specifically binding to human ErbB-3 and human c-Met comprising a first antigen-binding site that specifically binds to human ErbB-3 and a second antigen-binding site that specifically binds to human c-Met.

In one embodiment of the invention the antibody is a tetravalent, bispecific antibody specifically binding to human ErbB-3 and human c-Met comprising two antigen-binding sites that specifically bind to human ErbB-3 and two antigen-binding sites that specifically bind to human c-Met, wherein the antigen-binding sites that specifically bind to human c-Met inhibit the c-Met dimerisation (as described e.g. in WO 2009007427).

As used herein, “antibody” refers to a binding protein that comprises antigen-binding sites. The terms “binding site” or “antigen-binding site” as used herein denotes the region(s) of an antibody molecule to which a ligand actually binds and is derived from an antibody. The term “antigen-binding site” include antibody heavy chain variable domains (VH) and/or an antibody light chain variable domains (VL), or pairs of VH/VL, and can be derived from whole antibodies or antibody fragments such as single chain Fv, a VH domain and/or a VL domain, Fab, or (Fab)₂. In one embodiment of the current invention each of the antigen-binding sites comprises an antibody heavy chain variable domain (VH) and/or an antibody light chain variable domain (VL), and preferably is formed by a pair consisting of an antibody light chain variable domain (VL) and an antibody heavy chain variable domain (VH).

Further to antibody derived antigen-binding sites also binding peptides as described e.g. in Matzke, A., et al., Cancer Res 65 2005 (14). Jul. 15, 2005, can specifically bind to an antigen (e.g. c-Met). Thus a further aspect of the current invention is a bispecific binding molecule specifically binding to human ErbB-3 and human c-Met comprising a antigen-binding site that specifically binds to human ErbB-3 and a binding peptide that specifically binds to human c-Met. Thus a further aspect of the current invention is a bispecific binding molecule specifically binding to human ErbB-3 and human c-Met comprising a antigen-binding site that specifically binds to human c-Met and a binding peptide that specifically binds to human ErbB-3.

ErbB-3 (also known asV-erb-b2 erythroblastic leukemia viral oncogene homolog 3 (avian), ERBB3, HER3; SEQ ID NO:46) is membrane-bound protein which has a neuregulin binding domain but not an active kinase domain (Kraus, M. H., et al., Proc. Natl. Acad. Sci. U.S.A. 86 (1989) 9193-7; Plowman, G. D., et al., Proc. Natl. Acad. Sci. U.S.A. 87 (1990) 4905-9; Katoh, M., et al., Biochem. Biophys. Res. Commun. 192 (1993) 1189-97). It therefore can bind this ligand but not convey the signal into the cell through protein phosphorylation. However, it does form heterodimers with other EGF receptor family members which do have kinase activity. Heterodimerization leads to the activation of pathways which lead to cell proliferation or differentiation. Amplification of this gene and/or overexpression of its protein have been reported in numerous cancers, including prostate, bladder, and breast tumors. Alternate transcriptional splice variants encoding different isoforms have been characterized. One isoform lacks the intermembrane region and is secreted outside the cell. This form acts to modulate the activity of the membrane-bound form (Corfas, G., et al., 7 (6) (2004) 575-80). It is thought that ERBB3, when activated, becomes a substrate for dimerization and subsequent phosphorylation by ERBB1, ERBB2 and ERBB4. Like many of the receptor tyrosine-kinases, ERBB3 is activated by extracellular ligand. Ligands known to bind to ERBB3 include heregulin.

The antigen-binding site, and especially heavy chain variable domains (VH) and/or antibody light chain variable domains (VL), that specifically bind to human ErbB-3 can be derived a) from known anti-ErbB-3 antibodies as described e.g. WO 97/35885, WO 2007/077028 or WO 2008/100624 b) from new anti-ErbB-3 antibodies obtained by de novo immunization methods using inter alia either the human ErbB-3 protein or nucleic acid or fragments thereof or by phage display.

MET (mesenchymal-epithelial transition factor) is a proto-oncogene that encodes a protein MET, (also known as c-Met; hepatocyte growth factor receptor HGFR; HGF receptor; scatter factor receptor; SF receptor; SEQ. ID. NO:45) (Dean, M., et al., Nature 318 (1985) 385-8; Chan, A. M., et al., Oncogene 1 (1987) 229-33; Bottaro, D. P., et al., Science 251 (1991) 802-4; Naldini, L., et al., EMBO J. 10 (1991) 2867-78; Maulik, G., et al., Cytokine Growth Factor Rev. 13 (2002) 41-59). MET is a membrane receptor that is essential for embryonic development and wound healing. Hepatocyte growth factor (HGF) is the only known ligand of the MET receptor. MET is normally expressed by cells of epithelial origin, while expression of HGF is restricted to cells of mesenchymal origin. Upon HGF stimulation, MET induces several biological responses that collectively give rise to a program known as invasive growth. Abnormal MET activation in cancer correlates with poor prognosis, where aberrantly active MET triggers tumor growth, formation of new blood vessels (angiogenesis) that supply the tumor with nutrients, and cancer spread to other organs (metastasis). MET is deregulated in many types of human malignancies, including cancers of kidney, liver, stomach, breast, and brain. Normally, only stem cells and progenitor cells express MET, which allows these cells to grow invasively in order to generate new tissues in an embryo or regenerate damaged tissues in an adult. However, cancer stem cells are thought to hijack the ability of normal stem cells to express MET, and thus become the cause of cancer persistence and spread to other sites in the body.

The antigen-binding site, and especially heavy chain variable domains (VH) and/or antibody light chain variable domains (VL), that specifically bind to human c-Met can be derived a) from known anti-c-Met antibodies as describe e.g. in U.S. Pat. No. 5,686,292, U.S. Pat. No. 7,476,724, WO 2004072117, WO 2004108766, WO 2005016382, WO 2005063816, WO 2006015371, WO 2006104911, WO 2007126799, or WO 2009007427 b) from new anti-c-Met antibodies obtained e.g. by de novo immunization methods using inter alia either the human anti-c-Met protein or nucleic acid or fragments thereof or by phage display.

Another aspect of the invention is a method for the selection of a bispecific antibody according to the invention, comprising the steps of
a) measuring the internalization of ErbB-3 on A431 cells (ATCC No. CRL-1555) induced by a bispecific anti-ErbB-3/anti-c-Met antibody after 2 hours in a flow cytometry assay (FACS) as compared to the internalization of ErbB-3 in the absence of antibody
b) measuring the internalization of ErbB-3 on A431 cells (ATCC No. CRL-1555) in a flow cytometry assay (FACS) in the absence of antibody
c) selecting a bispecific antibody which shows an internalization of ErbB-3 of no more than 15% on A431 cells after 2 hours of antibody exposure as compared to the internalization of ErbB-3 in the absence of antibody.

In one embodiment a bispecific antibody which shows an internalization of ErbB-3 of no more than 10% is selected. In one embodiment a bispecific antibody which shows an internalization of ErbB-3 of no more than 7% is selected. In one embodiment a bispecific antibody which shows an internalization of ErbB-3 of no more than 5% is selected.

Another aspect of the invention is a method for the selection of a bispecific antibody according to the invention, comprising the steps of
a) measuring the internalization of ErbB-3 on A431 cells (ATCC No. CRL-1555) induced by a bispecific anti-ErbB-3/anti-c-Met antibody after 2 hours in a flow cytometry assay (FACS) as compared to the internalization of ErbB-3 in the absence of antibody
b) measuring the internalization of ErbB-3 on A431 cells (ATCC No. CRL-1555) induced by the corresponding monospecific anti-ErbB-3 antibody after 2 hours in a flow cytometry assay (FACS)
c) selecting a bispecific antibody which reduces the internalization of ErbB-3, compared to internalization of ErbB-3 induced by the corresponding monospecific parent ErbB-3 antibody, 50% or more (on A431 cells after 2 hours).

In one embodiment a bispecific antibody which reduces the internalization of ErbB-3, compared to internalization of ErbB-3 induced by the corresponding monospecific parent ErbB-3 antibody, 60% or more is selected. In one embodiment a bispecific antibody which reduces the internalization of ErbB-3, compared to internalization of ErbB-3 induced by the corresponding monospecific parent ErbB-3 antibody, 70% or more is selected. In one embodiment a bispecific antibody which reduces the internalization of ErbB-3, compared to internalization of ErbB-3 induced by the corresponding monospecific parent ErbB-3 antibody, 80% or more is selected.

Another aspect of the invention is a bispecific antibody specifically binding to human ErbB-3 and human c-Met comprising a first antigen-binding site that specifically binds to human ErbB-3 and a second antigen-binding site that specifically binds to human c-Met, characterized in that
i) the first antigen-binding site comprises in the heavy chain variable domain a CDR3H region of SEQ ID NO: 53, a CDR2H region of SEQ ID NO: 54, and a CDR1H region of SEQ ID NO:55, and in the light chain variable domain a CDR3L region of SEQ ID NO: 56, a CDR2L region of SEQ ID NO:57, and a CDR1L region of SEQ ID NO:58 or a CDR1L region of SEQ ID NO:59; and
   the second antigen-binding site comprises in the heavy chain variable domain a CDR3H region of SEQ ID NO: 66, a CDR2H region of, SEQ ID NO: 67, and a CDR1H region of SEQ ID NO: 68, and in the light chain variable domain a CDR3L region of SEQ ID NO: 69, a CDR2L region of SEQ ID NO: 70, and a CDR1L region of SEQ ID NO: 71.
ii) the first antigen-binding site comprises in the heavy chain variable domain a CDR3H region of SEQ ID NO: 60, a CDR2H region of SEQ ID NO: 61, and a CDR1H region of SEQ ID NO:62, and in the light chain variable domain a CDR3L region of SEQ ID NO: 63, a CDR2L region of SEQ ID NO:64, and a CDR1L region of SEQ ID NO:65 or a CDR1L region of SEQ ID NO:66; and
   the second antigen-binding site comprises in the heavy chain variable domain a CDR3H region of SEQ ID NO: 66, a CDR2H region of, SEQ ID NO: 67, and a CDR1H region of SEQ ID NO: 68, and in the light chain variable domain a CDR3L region of SEQ ID NO: 69, a CDR2L region of SEQ ID NO: 70, and a CDR1L region of SEQ ID NO: 71.
iii)

Another aspect of the invention is a bispecific antibody specifically binding to human ErbB-3 and human c-Met comprising a first antigen-binding site that specifically binds to human ErbB-3 and a second antigen-binding site that specifically binds to human c-Met, characterized in that

the first antigen-binding site comprises in the heavy chain variable domain a CDR3H region of SEQ ID NO: 53, a CDR2H region of SEQ ID NO: 54, and a CDR1H region of SEQ ID NO:55, and in the light chain variable domain a CDR3L region of SEQ ID NO: 56, a CDR2L region of SEQ ID NO:57, and a CDR1L region of SEQ ID NO:58 or a CDR1L region of SEQ ID NO:59; and
the second antigen-binding site comprises in the heavy chain variable domain a CDR3H region of SEQ ID NO: 66, a CDR2H region of, SEQ ID NO: 67, and a CDR1H region of SEQ ID NO: 68, and in the light chain variable domain a CDR3L region of SEQ ID NO: 69, a CDR2L region of SEQ ID NO: 70, and a CDR1L region of SEQ ID NO: 71.

Another aspect of the invention is a bispecific antibody specifically binding to human ErbB-3 and human c-Met comprising a first antigen-binding site that specifically binds to human ErbB-3 and a second antigen-binding site that specifically binds to human c-Met, characterized in that

the first antigen-binding site comprises in the heavy chain variable domain a CDR3H region of SEQ ID NO: 60, a CDR2H region of SEQ ID NO: 61, and a CDR1H region of SEQ ID NO:62, and in the light chain variable domain a CDR3L region of SEQ ID NO: 63, a CDR2L region of SEQ ID NO:64, and a CDR1L region of SEQ ID NO:65 or a CDR1L region of SEQ ID NO:66; and

the second antigen-binding site comprises in the heavy chain variable domain a CDR3H region of SEQ ID NO: 66, a CDR2H region of, SEQ ID NO: 67, and a CDR1H region of SEQ ID NO: 68, and in the light chain variable domain a CDR3L region of SEQ ID NO: 69, a CDR2L region of SEQ ID NO: 70, and a CDR1L region of SEQ ID NO: 71.

The bispecific antibody is preferably, characterized in that
i) the first antigen-binding site comprises as heavy chain variable domain SEQ ID NO: 47, and as light chain variable domain SEQ ID NO: 48, and the second antigen-binding site comprises as heavy chain variable domain SEQ ID NO: 3, and as light chain variable domain a SEQ ID NO: 4;
ii) the first antigen-binding site comprises as heavy chain variable domain SEQ ID NO: 49, and as light chain variable domain SEQ ID NO: 50, and the second antigen-binding site comprises as heavy chain variable domain SEQ ID NO: 3, and as light chain variable domain a SEQ ID NO: 4;
iii) the first antigen-binding site comprises as heavy chain variable domain SEQ ID NO: 49, and as light chain variable domain SEQ ID NO: 51, and the second antigen-binding site comprises as heavy chain variable domain SEQ ID NO: 3, and as light chain variable domain a SEQ ID NO: 4;
iv) the first antigen-binding site comprises as heavy chain variable domain SEQ ID NO: 49, and as light chain variable domain SEQ ID NO: 52, and the second antigen-binding site comprises as heavy chain variable domain SEQ ID NO: 3, and as light chain variable domain a SEQ ID NO: 4; or
v) the first antigen-binding site comprises as heavy chain variable domain SEQ ID NO: 1, and as light chain variable domain SEQ ID NO: 2, and the second antigen-binding site comprises as heavy chain variable domain SEQ ID NO: 3, and as light chain variable domain a SEQ ID NO: 4; or

Preferably the bispecific antibody is characterized in that

the first antigen-binding site comprises as heavy chain variable domain SEQ ID NO: 49, and as light chain variable domain SEQ ID NO: 51, and the second antigen-binding site comprises as heavy chain variable domain SEQ ID NO: 3, and as light chain variable domain a SEQ ID NO: 4.

Antibody specificity refers to selective recognition of the antibody for a particular epitope of an antigen. Natural antibodies, for example, are monospecific. “Bispecific antibodies” according to the invention are antibodies which have two different antigen-binding specificities. Where an antibody has more than one specificity, the recognized epitopes may be associated with a single antigen or with more than one antigen. Antibodies of the present invention are specific for two different antigens, i.e. ErbB-3 as first antigen and c-Met as second antigen.

The term “monospecific” antibody as used herein denotes an antibody that has one or more binding sites each of which bind to the same epitope of the same antigen.

The term “valent” as used within the current application denotes the presence of a specified number of binding sites in an antibody molecule. As such, the terms “bivalent”, “tetravalent”, and “hexavalent” denote the presence of two binding site, four binding sites, and six binding sites, respectively, in an antibody molecule. The bispecific antibodies according to the invention are at least “bivalent” and may be “trivalent” or “multivalent” (e.g.(“tetravalent” or “hexavalent”).

An antigen-binding site of an antibody of the invention can contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences. Also included within the scope of the invention are functional antigen binding sites comprised of fewer CDRs (i.e., where binding specificity is determined by three, four or five CDRs). For example, less than a complete set of 6 CDRs may be sufficient for binding. In some cases, a VH or a VL domain will be sufficient.

IgG like bispecific, bivalent antibodies against human ErbB-3 and human c-Met comprising the immunoglobulin constant regions can be used as described e.g. in EP Appl. No. 07024867.9, EP Appl. No. 07024864.6, EP Appl. No. 07024865.3 or Ridgway, J. B., Protein Eng. 9 (1996) 617-621; WO 96/027011; Merchant, A. M, et al., Nature Biotech 16 (1998) 677-681; Atwell, S., et al., J. Mol. Biol. 270 (1997) 26-35 and EP 1 870 459A1.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties according to the invention, especially in regard to C1q binding and/or Fc receptor (FcR) binding. Such chimeric antibodies are also referred to as “class-switched antibodies.”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See, e.g., Morrison, S. L., et al., Proc. Natl. Acad. Sci. USA 81 (1984) 6851-6855; U.S. Pat. No. 5,202,238 and U.S. Pat. No. 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric antibodies. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Brüggemann, M. D., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole, A., et al. and Boerner, P., et al. are also available for the preparation of human monoclonal antibodies (Cole, A., et al., Monoclonal Antibodies and Cancer Therapy, Liss, A. L., p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation).

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The “variable domain” (variable domain of a light chain (VL), variable region of a heavy chain (VH) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies according to the invention and therefore provide a further object of the invention.

The terms “hypervariable region” or “antigen-binding portion of an antibody or an antigen binding site” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. CDRs on each chain are separated by such framework amino acids. Especially, CDR3 of the heavy chain is the region which contributes most to antigen binding. CDR and FR regions are determined according to the standard definition of Kabat et al., Sequences of Proteins of Immunological Interest, 5th ed., Public Health Service, National Institutes of Health, Bethesda, Md. (1991).

As used herein, the term “binding” or “specifically binding” refers to the binding of the antibody to an epitope of the antigen (either human ErbB-3 or human c-Met) in an in vitro assay, preferably in an plasmon resonance assay (BIAcore, GE-Healthcare Uppsala, Sweden) with purified wild-type antigen. The affinity of the binding is defined by the terms ka (rate constant for the association of the antibody from the antibody/antigen complex), k_{D} (dissociation constant), and K_{D} (k_{D}/ka). Binding or specifically binding means a binding affinity (K_{D}) of 10⁻⁸ mol/l or less, preferably 10⁻⁹ M to 10⁻¹³ mol/l. Thus, an bispecific <ErbB3-c-Met> antibody according to the invention is specifically binding to each antigen for which it is specific with a binding affinity (K_{D}) of 10⁻⁸ mol/l or less, preferably 10⁻⁹ M to 10⁻¹³ mol/l.

Binding of the antibody to the FcγRIII can be investigated by a BIAcore assay (GE-Healthcare Uppsala, Sweden). The affinity of the binding is defined by the terms ka (rate constant for the association of the antibody from the antibody/antigen complex), k_{D} (dissociation constant), and K_{D} (k_{D}/ka).

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

In certain embodiments, an antibody is said to specifically bind an antigen when it preferentially recognizes its target antigen in a complex mixture of proteins and/or macromolecules.

The term “constant region” as used within the current applications denotes the sum of the domains of an antibody other than the variable region. The constant region is not involved directly in binding of an antigen, but exhibits various effector functions. Depending on the amino acid sequence of the constant region of their heavy chains, antibodies are divided in the classes: IgA, IgD, IgE, IgG and IgM, and several of these may be further divided into subclasses, such as IgG1, IgG2, IgG3, and IgG4, IgA1 and IgA2. The heavy chain constant regions that correspond to the different classes of antibodies are called α, δ, ε, γ, and μ, respectively. The light chain constant regions which can be found in all five antibody classes are called κ (kappa) and λ (lambda).

The term “constant region derived from human origin” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., (see e.g. Johnson, G. and Wu, T. T., Nucleic Acids Res. 28 (2000) 214-218; Kabat, E. A., et al., Proc. Natl. Acad. Sci. USA 72 (1975) 2785-2788).

In one embodiment the bispecific antibodies according to the invention comprise a constant region of IgG1 or IgG3 subclass (preferably of IgG1 subclass), which is preferably derived from human origin. In one embodiment the bispecific antibodies according to the invention comprise a Fc part of IgG1 or IgG3 subclass (preferably of IgG1 subclass), which is preferably derived from human origin.

The constant region of an antibody is directly involved in ADCC (antibody-dependent cell-mediated cytotoxicity) and CDC (complement-dependent cytotoxicity). Complement activation (CDC) is initiated by binding of complement factor C1q to the constant region of most IgG antibody subclasses. Binding of C1q to an antibody is caused by defined protein-protein interactions at the so called binding site. Such constant region binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such constant region binding sites are, e.g., characterized by the amino acids L234, L235, D270, N297, E318, K320, K322, P331, and P329 (numbering according to EU index of Kabat).

The term “antibody-dependent cellular cytotoxicity (ADCC)” refers to lysis of human target cells by an antibody according to the invention in the presence of effector cells. ADCC is measured preferably by the treatment of a preparation of ErbB3 and c-Met expressing cells with an antibody according to the invention in the presence of effector cells such as freshly isolated PBMC or purified effector cells from buffy coats, like monocytes or natural killer (NK) cells or a permanently growing NK cell line. The term “complement-dependent cytotoxicity (CDC)” denotes a process initiated by binding of complement factor C1q to the Fc part of most IgG antibody subclasses. Binding of C1q to an antibody is caused by defined protein-protein interactions at the so called binding site. Such Fc part binding sites are known in the state of the art (see above). Such Fc part binding sites are, e.g., characterized by the amino acids L234, L235, D270, N297, E318, K320, K322, P331, and P329 (numbering according to EU index of Kabat). Antibodies of subclass IgG1, IgG2, and IgG3 usually show complement activation including C1q and C3 binding, whereas IgG4 does not activate the complement system and does not bind C1q and/or C3.

Cell-mediated effector functions of monoclonal antibodies can be enhanced by engineering their oligosaccharide component as described in Umana, P., et al., Nature Biotechnol. 17 (1999) 176-180, and U.S. Pat. No. 6,602,684. IgG1 type antibodies, the most commonly used therapeutic antibodies, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A., and Morrison, S. L., Trends Biotechnol. 15 (1997) 26-32). Umana, P., et al. Nature Biotechnol. 17 (1999) 176-180 and WO 99/54342 showed that overexpression in Chinese hamster ovary (CHO) cells of β(1,4)-N-acetylglucosaminyltransferase III (“GnTIII”), a glycosyltransferase catalyzing the formation of bisected oligosaccharides, significantly increases the in vitro ADCC activity of antibodies. Alterations in the composition of the Asn297 carbohydrate or its elimination affect also binding to FcγR and C1q (Umana, P., et al., Nature Biotechnol. 17 (1999) 176-180; Davies, J., et al., Biotechnol. Bioeng. 74 (2001) 288-294; Mimura, Y., et al., J. Biol. Chem. 276 (2001) 45539-45547; Radaev, S., et al., J. Biol. Chem. 276 (2001) 16478-16483; Shields, R. L., et al., J. Biol. Chem. 276 (2001) 6591-6604; Shields, R. L., et al., J. Biol. Chem. 277 (2002) 26733-26740; Simmons, L. C., et al., J. Immunol. Methods 263 (2002) 133-147).

Methods to enhance cell-mediated effector functions of monoclonal antibodies by reducing the amount of fucose are described e.g. in WO 2005/018572, WO 2006/116260, WO 2006/114700, WO 2004/065540, WO 2005/011735, WO 2005/027966, WO 1997/028267, US 2006/0134709, US 2005/0054048, US 2005/0152894, WO 2003/035835, WO 2000/061739, Niwa, R., et al., J. Immunol. Methods 306 (2005) 151-160; Shinkawa, T., et al, J Biol Chem, 278 (2003) 3466-3473; WO 03/055993 or US 2005/0249722.

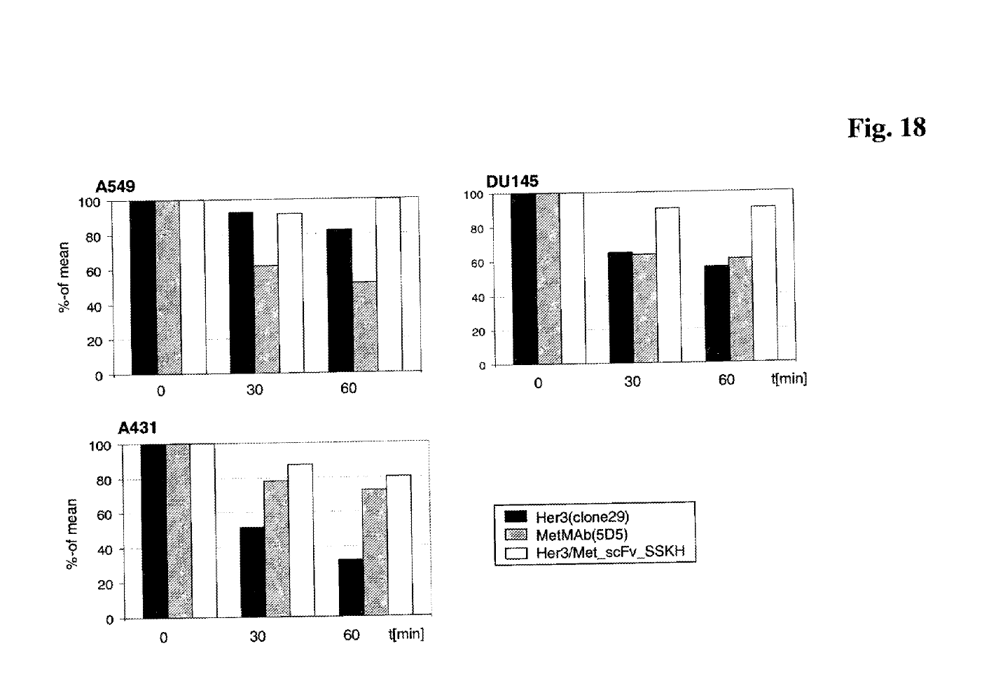

Surprisingly the bispecific <ErbB3-c-Met> antibodies according to the invention show a strong reduction of the internalization of ErbB-3 receptor compared to their parent <ErbB3> and/or <c-Met> antibodies. (see FIG. 18 Example 8 and Table). Therefore in one preferred embodiment of the invention, the bispecific antibody is glycosylated (IgG1 or IgG3 subclass) with a sugar chain at Asn297 whereby the amount of fucose within the sugar chain is 65% or lower (Numbering according to Kabat). In another embodiment is the amount of fucose within the sugar chain is between 5% and 65%, preferably between 20% and 40%. “Asn297” according to the invention means amino acid asparagine located at about position 297 in the Fc region. Based on minor sequence variations of antibodies, Asn297 can also be located some amino acids (usually not more than ±3 amino acids) upstream or downstream of position 297, i.e. between position 294 and 300. Such glycoengineered antibodies are also refer to as afousylated antibodies herein.

Glycosylation of human IgG1 or IgG3 occurs at Asn297 as core fucosylated biantennary complex oligosaccharide glycosylation terminated with up to two Gal residues. Human constant heavy chain regions of the IgG1 or IgG3 subclass are reported in detail by Kabat, E. A., et al., Sequences of Proteins of Immunological Interest, 5th Ed. Public Health Service, National Institutes of Health, Bethesda, Md. (1991), and by Brüggemann, M., et al., J. Exp. Med. 166 (1987) 1351-1361; Love, T. W., et al., Methods Enzymol. 178 (1989) 515-527. These structures are designated as G0, G1 (α-1,6- or α-1,3-), or G2 glycan residues, depending from the amount of terminal Gal residues (Raju, T. S., Bioprocess Int. 1 (2003) 44-53). CHO type glycosylation of antibody Fc parts is e.g. described by Routier, F. H., Glycoconjugate J. 14 (1997) 201-207. Antibodies which are recombinantly expressed in non-glycomodified CHO host cells usually are fucosylated at Asn297 in an amount of at least 85%. The modified oligosaccharides of the full length parent antibody may be hybrid or complex. Preferably the bisected, reduced/not-fucosylated oligosaccharides are hybrid. In another embodiment, the bisected, reduced/not-fucosylated oligosaccharides are complex.

According to the invention “amount of fucose” means the amount of the sugar within the sugar chain at Asn297, related to the sum of all glycostructures attached to Asn297 (e.g. complex, hybrid and high mannose structures) measured by MALDI-TOF mass spectrometry and calculated as average value. The relative amount of fucose is the percentage of fucose-containing structures related to all glycostructures identified in an N-Glycosidase F treated sample (e.g. complex, hybrid and oligo- and high-mannose structures, resp.) by MALDI-TOF (for a detailed procedure to determine the amount of fucose, see Example 14).

The afucosylated bispecific antibody according to the invention can be expressed in a glycomodified host cell engineered to express at least one nucleic acid encoding a polypeptide having GnTIII activity in an amount sufficient to partially fucosylate the oligosaccharides in the Fc region. In one embodiment, the polypeptide having GnTIII activity is a fusion polypeptide. Alternatively α1,6-fucosyltransferase activity of the host cell can be decreased or eliminated according to U.S. Pat. No. 6,946,292 to generate glycomodified host cells. The amount of antibody fucosylation can be predetermined e.g. either by fermentation conditions (e.g. fermentation time) or by combination of at least two antibodies with different fucosylation amount. Such afucosylated antibodies and respective glycoengineering methods are described in WO 2005/044859, WO 2004/065540, WO2007/031875, Umana, P., et al., Nature Biotechnol. 17 (1999) 176-180, WO 99/154342, WO 2005/018572, WO 2006/116260, WO 2006/114700, WO 2005/011735, WO 2005/027966, WO 97/028267, US 2006/0134709, US 2005/0054048, US 2005/0152894, WO 2003/035835, WO 2000/061739. These glycoengineered antibodies have an increased ADCC. Other glycoengineering methods yielding afucosylated antibodies according to the invention are described e.g. in Niwa, R., et al., J. Immunol. Methods 306 (2005) 151-160; Shinkawa, T., et al, J Biol Chem, 278 (2003) 3466-3473; WO 03/055993 or US 2005/0249722.

One embodiment is a method of preparation of the bispecific antibody of IgG1 or IgG3 subclass which is glycosylated (of) with a sugar chain at Asn297 whereby the amount of fucose within the sugar chain is 65% or lower, using the procedure described in WO 2005/044859, WO 2004/065540, WO 2007/031875, Umana, P., et al., Nature Biotechnol. 17 (1999) 176-180, WO 99/154342, WO 2005/018572, WO 2006/116260, WO 2006/114700, WO 2005/011735, WO 2005/027966, WO 97/028267, US 2006/0134709, US 2005/0054048, US 2005/0152894, WO 2003/035835 or WO 2000/061739.

One embodiment is a method of preparation of the bispecific antibody of IgG1 or IgG3 subclass which is glycosylated (of) with a sugar chain at Asn297 whereby the amount of fucose within the sugar chain is 65% or lower, using the procedure described in Niwa, R., et al., J. Immunol. Methods 306 (2005) 151-160; Shinkawa, T., et al, J Biol Chem, 278 (2003) 3466-3473; WO 03/055993 or US 2005/0249722.

In one embodiment the antibodies according to the invention inhibit HGF-induced c-Met receptor phosphorylation in A549 cells (as described in Example 2).

In one embodiment the antibodies according to the invention inhibit HRG(Herregulin)-induced Her3 receptor phosphorylation in MCF7 cells by at least 70% at a concentration of 1 μg/ml (as described in Example 3) (compared to HRG as control).

In one embodiment the antibodies according to the invention inhibit HGF-induced proliferation of HUVEC cells by at least 40% at a concentration of 12.5 μg/ml (as described in Example 4) (compared to HGF alone as a control).

### Bispecific Antibody Formats

Antibodies of the present invention have two or more binding sites and are bispecific. That is, the antibodies may be bispecific even in cases where there are more than two binding sites (i.e. that the antibody is trivalent or multivalent). Bispecific antibodies of the invention include, for example, multivalent single chain antibodies, diabodies and triabodies, as well as antibodies having the constant domain structure of full length antibodies to which further antigen-binding sites (e.g., single chain Fv, a VH domain and/or a VL domain, Fab, or (Fab)2,) are linked via one or more peptide-linkers. The antibodies can be full length from a single species, or be chimerized or humanized. For an antibody with more than two antigen binding sites, some binding sites may be identical, so long as the protein has binding sites for two different antigens. That is, whereas a first binding site is specific for a ErbB-3, a second binding site is specific for c-Met, and vice versa.

In a preferred embodiment the bispecific antibody specifically binding to human ErbB-3 and human c-Met according to the invention comprises the Fc region of an antibody. Such an antibody retains the properties of which means that In one embodiment a full length an

[symbol]

### Bivalent Bispecific Formats

Bispecific, bivalent antibodies against human ErbB-3 and human c-Met comprising the immunoglobulin constant regions can be used as described e.g. in WO 2009/080251, WO 2009/080252, WO 2009/080253 or Ridgway, J. B., Protein Eng. 9 (1996) 617-621; WO 96/027011; Merchant, A. M., et al., Nature Biotech 16 (1998) 677-681; Atwell, S., et al., J. Mol. Biol. 270 (1997) 26-35 and EP 1 870 459A1.

Thus in one embodiment of the invention the bispecific <ErbB3-c-Met> antibody according to the invention is a bivalent, bispecific antibody, comprising:

a) the light chain and heavy chain of a full length antibody specifically binding to human ErbB-3; and
b) the light chain and heavy chain of a full length antibody specifically binding to human c-Met,
wherein the constant domains CL and CH1, and/or the variable domains VL and VH are replaced by each other.

In another embodiment of the invention the bispecific <ErbB3-c-Met> antibody according to the invention is a bivalent, bispecific antibody, comprising:

a) the light chain and heavy chain of a full length antibody specifically binding to human c-Met; and
b) the light chain and heavy chain of a full length antibody specifically binding to human ErbB-3,
wherein the constant domains CL and CH1, and/or the variable domains VL and VH are replaced by each other.

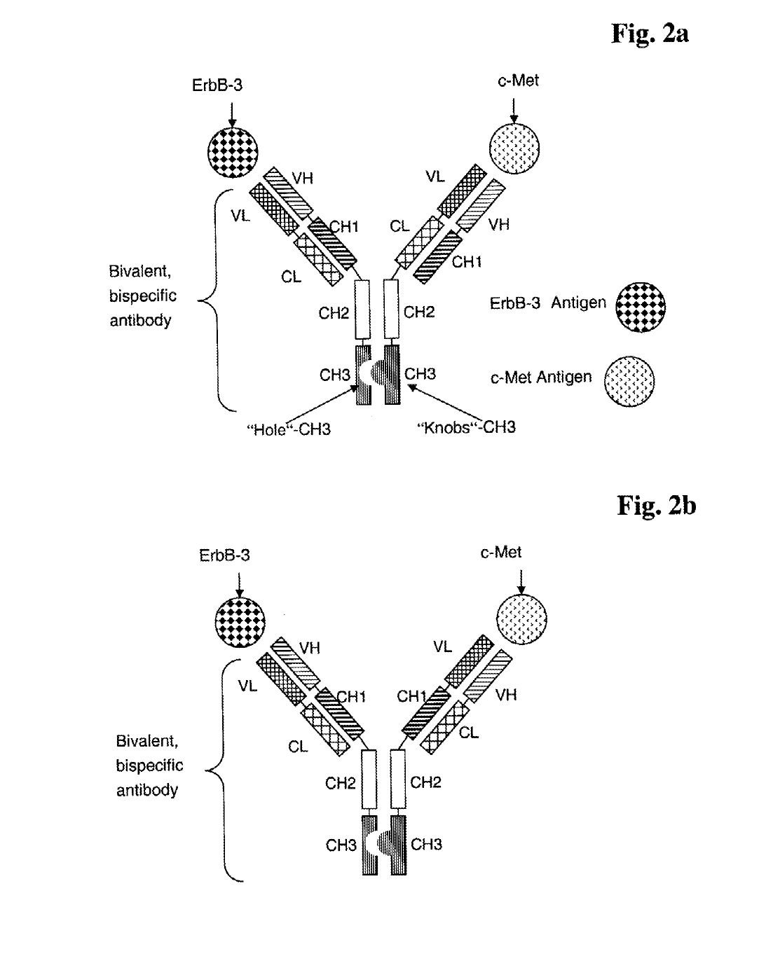

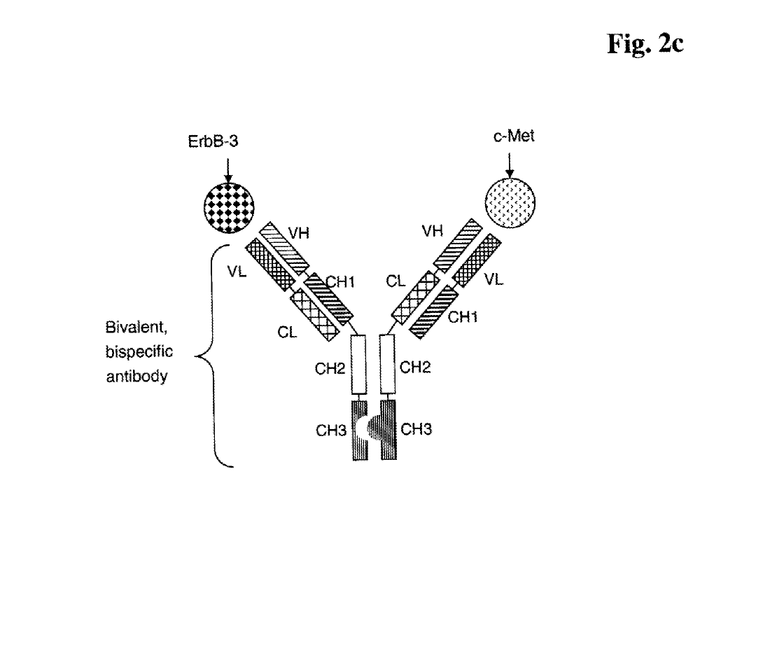

For an exemplary schematic structure with the “knob-into-holes” technology as described below see FIG. 2a-c.

To improve the yields of such hetrodimeric bivalent, bispecific anti-ErbB-3/anti-c-Met antibodies, the CH3 domains of the full length antibody can be altered by the “knob-into-holes” technology which is described in detail with several examples in e.g. WO 96/027011, Ridgway, J. B., et al., Protein Eng 9 (1996) 617-621; and Merchant, A. M., et al., Nat Biotechnol 16 (1998) 677-681. In this method the interaction surfaces of the two CH3 domains are altered to increase the heterodimerisation of both heavy chains containing these two CH3 domains. Each of the two CH3 domains (of the two heavy chains) can be the “knob”, while the other is the “hole”. The introduction of a disulfide bridge stabilizes the heterodimers (Merchant, A. M., et al., Nature Biotech 16 (1998) 677-681; Atwell, S., et al. J. Mol. Biol. 270 (1997) 26-35) and increases the yield.

Thus in one aspect of the invention the bivalent, bispecific antibody is further is characterized in that the CH3 domain of one heavy chain and the CH3 domain of the other heavy chain each meet at an interface which comprises an original interface between the antibody CH3 domains;

wherein the interface is altered to promote the formation of the bivalent, bispecific antibody,
wherein the alteration is characterized in that:
a) the CH3 domain of one heavy chain is altered,
so that within the original interface the CH3 domain of one heavy chain that meets the original interface of the CH3 domain of the other heavy chain within the bivalent, bispecific antibody,
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and
b) the CH3 domain of the other heavy chain is altered,
so that within the original interface of the second CH3 domain that meets the original interface of the first CH3 domain within the bivalent, bispecific antibody
an amino acid residue is replaced with an amino acid residue having a smaller side chain volume, thereby generating a cavity within the interface of the second CH3 domain within which a protuberance within the interface of the first CH3 domain is positionable.

Preferably the amino acid residue having a larger side chain volume is selected from the group consisting of arginine (R), phenylalanine (F), tyrosine (Y), tryptophan (W).

Preferably the amino acid residue having a smaller side chain volume is selected from the group consisting of alanine (A), serine (S), threonine (T), valine (V).

In one aspect of the invention both CH3 domains are further altered by the introduction of cysteine (C) as amino acid in the corresponding positions of each CH3 domain such that a disulfide bridge between both CH3 domains can be formed.

In a preferred embodiment, the bivalent, bispecific comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M., et al., Nature Biotech 16 (1998) 677-681) e.g. by introducing a Y349C mutation into the CH3 domain of the “knobs chain” and a E356C mutation or a S354C mutation into the CH3 domain of the “hole chain”. Thus in a another preferred embodiment, the bivalent, bispecific antibody comprises Y349C, T366W mutations in one of the two CH3 domains and E356C, T366S, L368A, Y407V mutations in the other of the two CH3 domains or the bivalent, bispecific antibody comprises Y349C, T366W mutations in one of the two CH3 domains and S354C, T366S, L368A, Y407V mutations in the other of the two CH3 domains (the additional Y349C mutation in one CH3 domain and the additional E356C or S354C mutation in the other CH3 domain forming a interchain disulfide bridge) (numbering always according to EU index of Kabat). But also other knobs-in-holes technologies as described by EP 1 870 459 A1, can be used alternatively or additionally. A preferred example for the bivalent, bispecific antibody are R409D; K370E mutations in the CH3 domain of the “knobs chain” and D399K; E357K mutations in the CH3 domain of the “hole chain” (numbering always according to EU index of Kabat).

In another preferred embodiment the bivalent, bispecific antibody comprises a T366W mutation in the CH3 domain of the “knobs chain” and T366S, L368A, Y407V mutations in the CH3 domain of the “hole chain” and additionally R409D; K370E mutations in the CH3 domain of the “knobs chain” and D399K; E357K mutations in the CH3 domain of the “hole chain”.

In another preferred embodiment the bivalent, bispecific antibody comprises Y349C, T366W mutations in one of the two CH3 domains and S354C, T366S, L368A, Y407V mutations in the other of the two CH3 domains or the bivalent, bispecific antibody comprises Y349C, T366W mutations in one of the two CH3 domains and S354C, T366S, L368A, Y407V mutations in the other of the two CH3 domains and additionally R409D; K370E mutations in the CH3 domain of the “knobs chain” and D399K; E357K mutations in the CH3 domain of the “hole chain”.

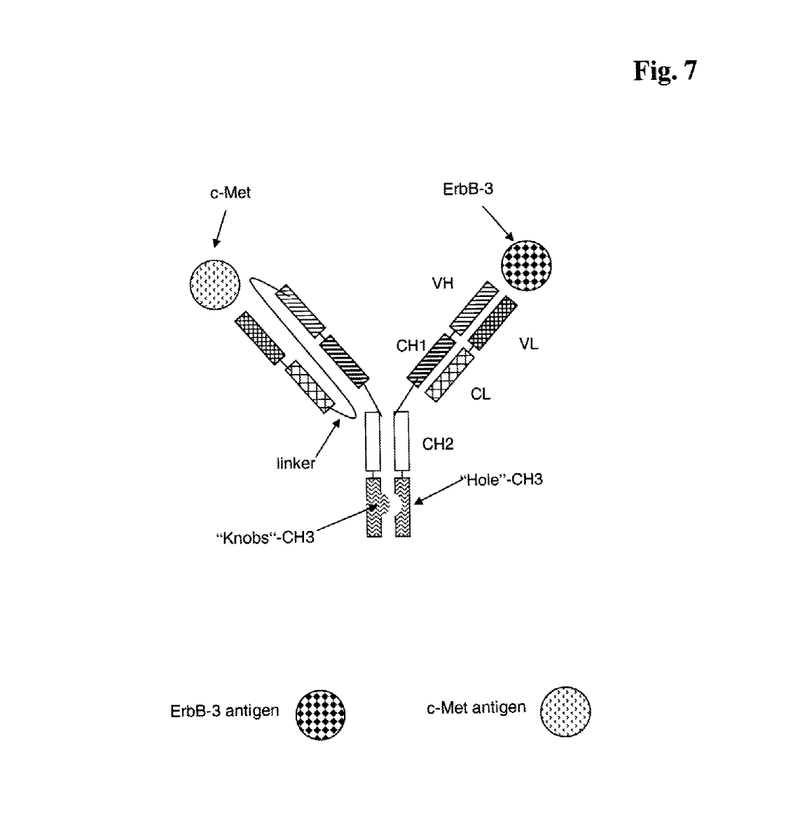

Examples of bivalent, bispecific antibody in a format described in Table 5 and FIG. 7 which were expressed and purified are described in the Examples below (See e.g. Table 5 and FIG. 7).

### Trivalent Bispecific Formats

Another preferred aspect of the current invention is a trivalent, bispecific antibody comprising

a) a full length antibody specifically binding to human ErbB-3 and consisting of two antibody heavy chains and two antibody light chains; and
b) one single chain Fab fragment specifically binding to human c-Met,
wherein the single chain Fab fragment under b) is fused to the full length antibody under a) via a peptide connector at the C- or N-terminus of the heavy or light chain of the full length antibody.

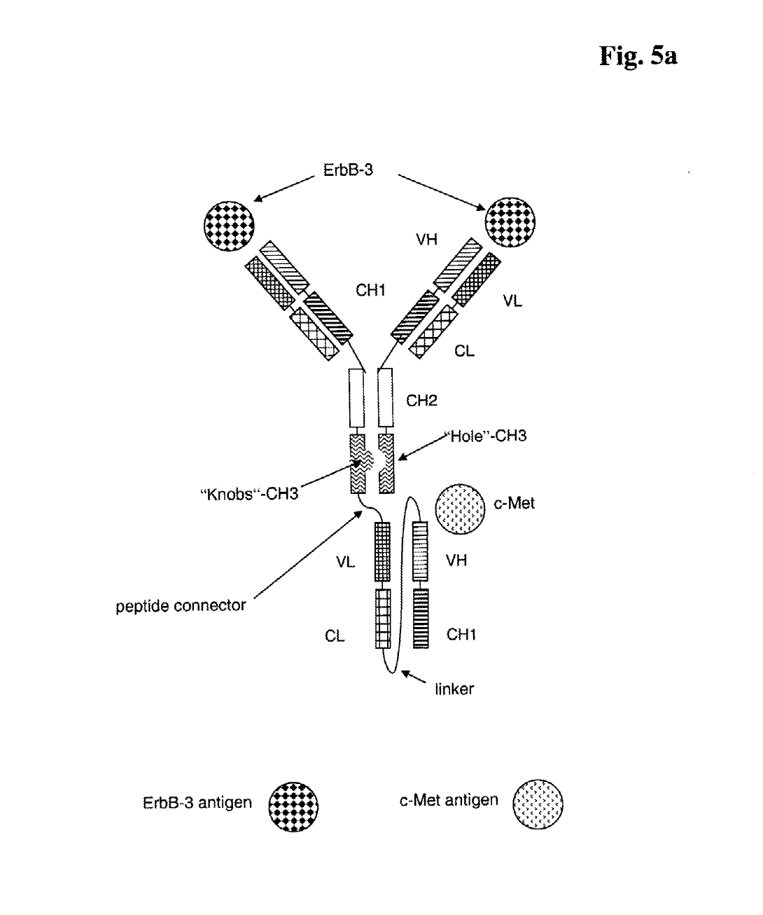

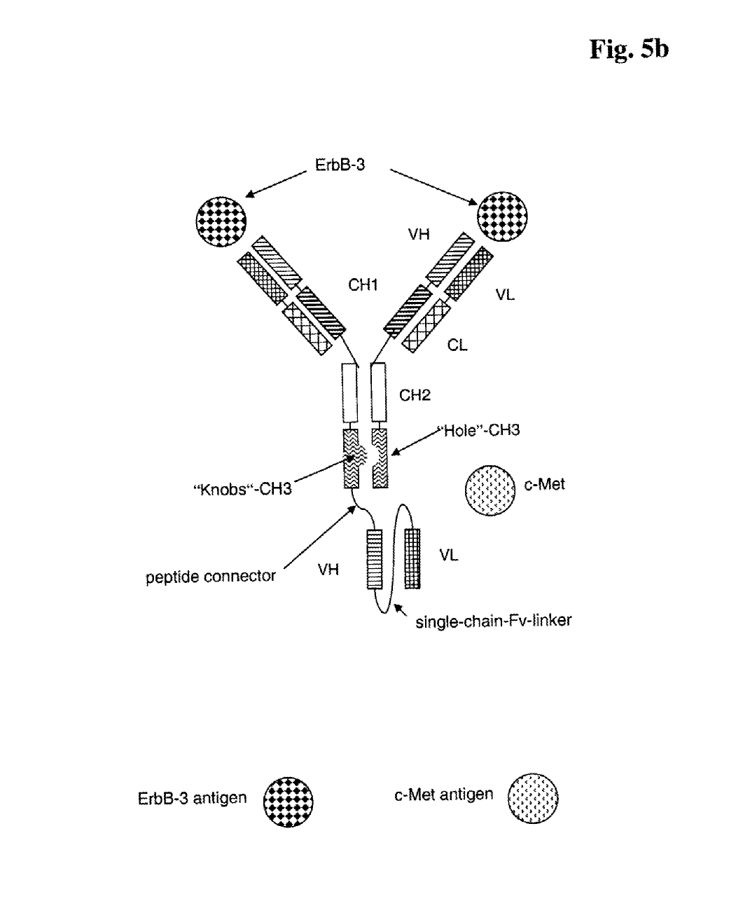

For an exemplary schematic structure with the “knob-into-holes” technology as described below see FIG. 5a.

Another preferred aspect of the current invention is a trivalent, bispecific antibody comprising

a) a full length antibody specifically binding to human ErbB-3 and consisting of two antibody heavy chains and two antibody light chains; and
b) one single chain Fv fragment specifically binding to human c-Met,
wherein the single chain Fv fragment under b) is fused to the full length antibody under a) via a peptide connector at the C- or N-terminus of the heavy or light chain of the full length antibody.

For an exemplary schematic structure with the “knob-into-holes” technology as described below see FIG. 5b.

Accordingly the corresponding trivalent, bispecific antibody with the scFv-Ab-nomenclature in Table 1 were expressed and purified (see in the Examples below)

In one preferred embodiment the single chain Fab or Fv fragments binding human c-Met are fused to the full length antibody via a peptide connector at the C-terminus of the heavy chains of the full length antibody.

Another preferred aspect of the current invention is a trivalent, bispecific antibody comprising

a) a full length antibody specifically binding to human ErbB-3 and consisting of two antibody heavy chains and two antibody light chains;
b) a polypeptide consisting of
ba) an antibody heavy chain variable domain (VH); or
bb) an antibody heavy chain variable domain (VH) and an antibody constant domain 1 (CH1),
wherein the polypeptide is fused with the N-terminus of the VH domain via a peptide connector to the C-terminus of one of the two heavy chains of the full length antibody
c) a polypeptide consisting of
ca) an antibody light chain variable domain (VL), or
cb) an antibody light chain variable domain (VL) and an antibody light chain constant domain (CL);
wherein the polypeptide is fused with the N-terminus of the VL domain via a peptide connector to the C-terminus of the other of the two heavy chains of the full length antibody;
and wherein the antibody heavy chain variable domain (VH) of the polypeptide under b) and the antibody light chain variable domain (VL) of the polypeptide under c) together form an antigen-binding site specifically binding to human c-Met.

Preferably the peptide connectors under b) and c) are identical and are a peptide of at least 25 amino acids, preferably between 30 and 50 amino acids.

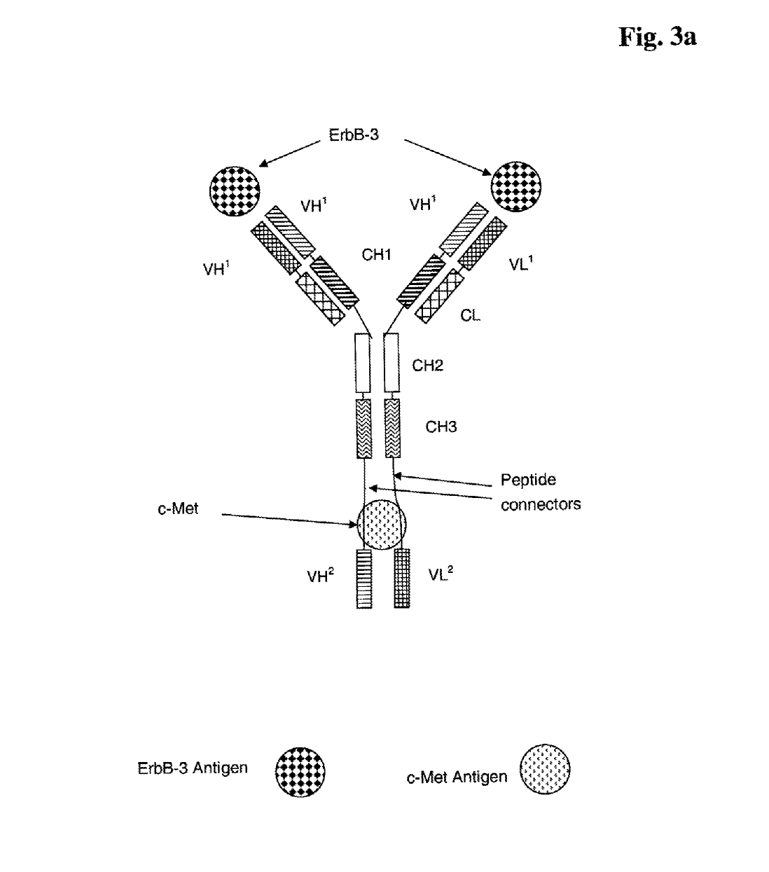

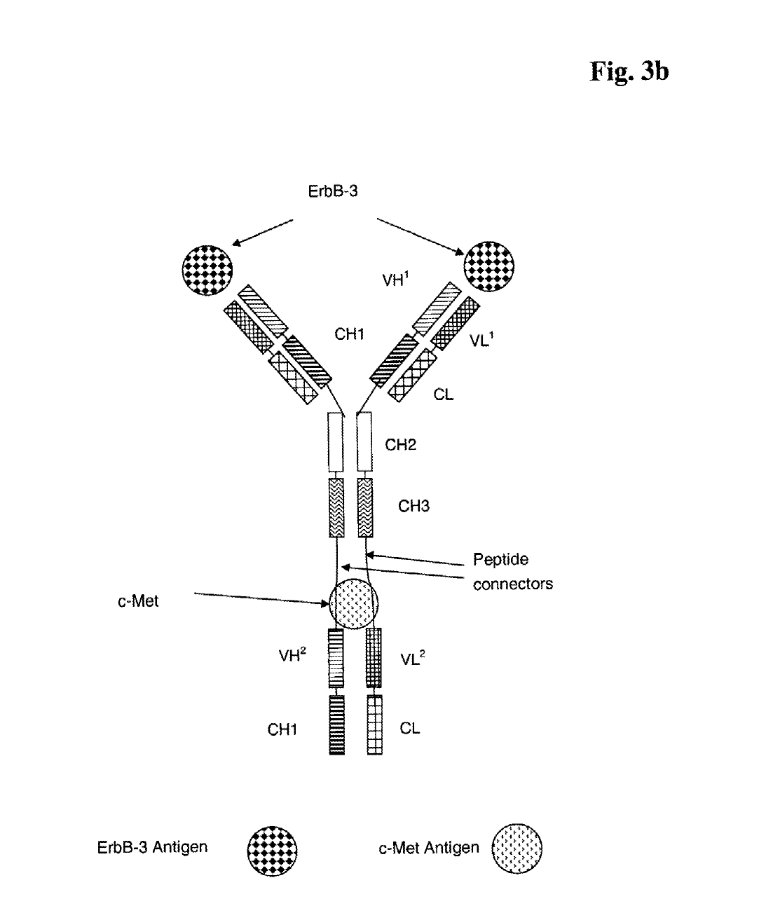

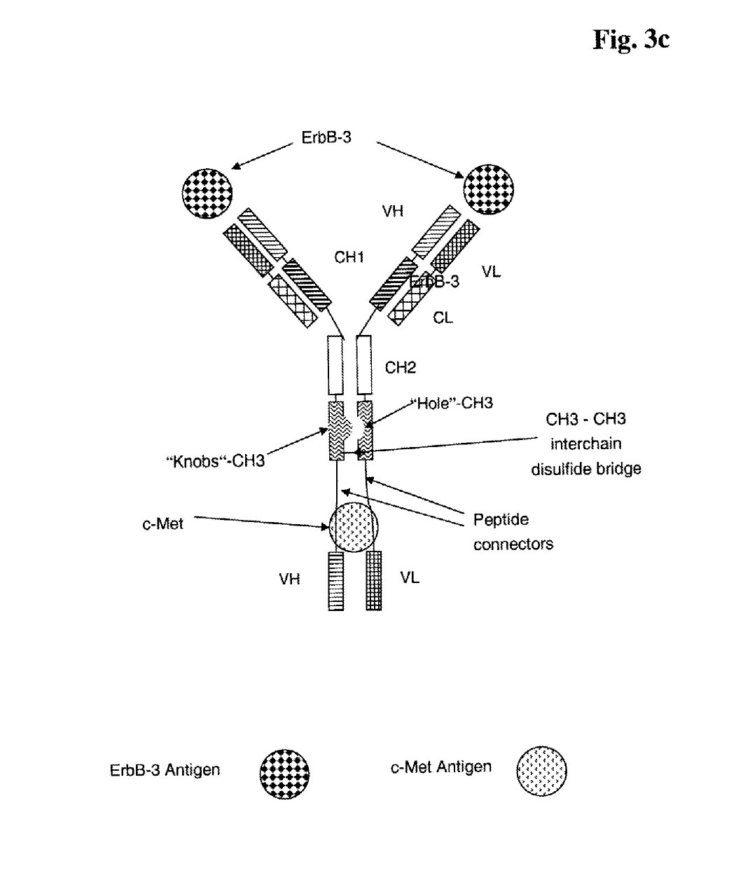

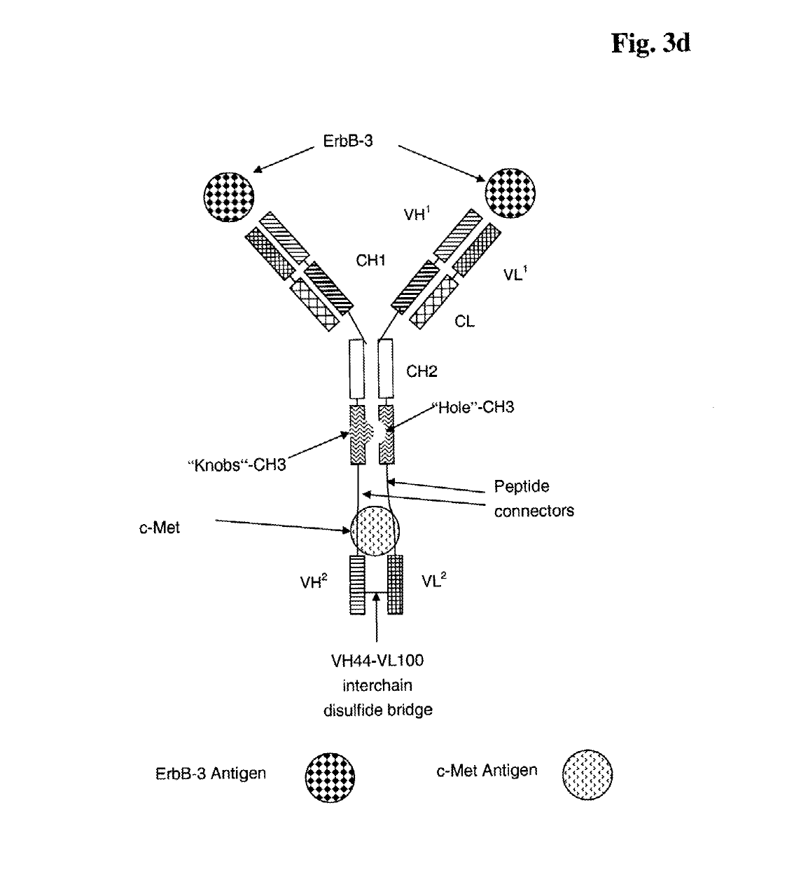

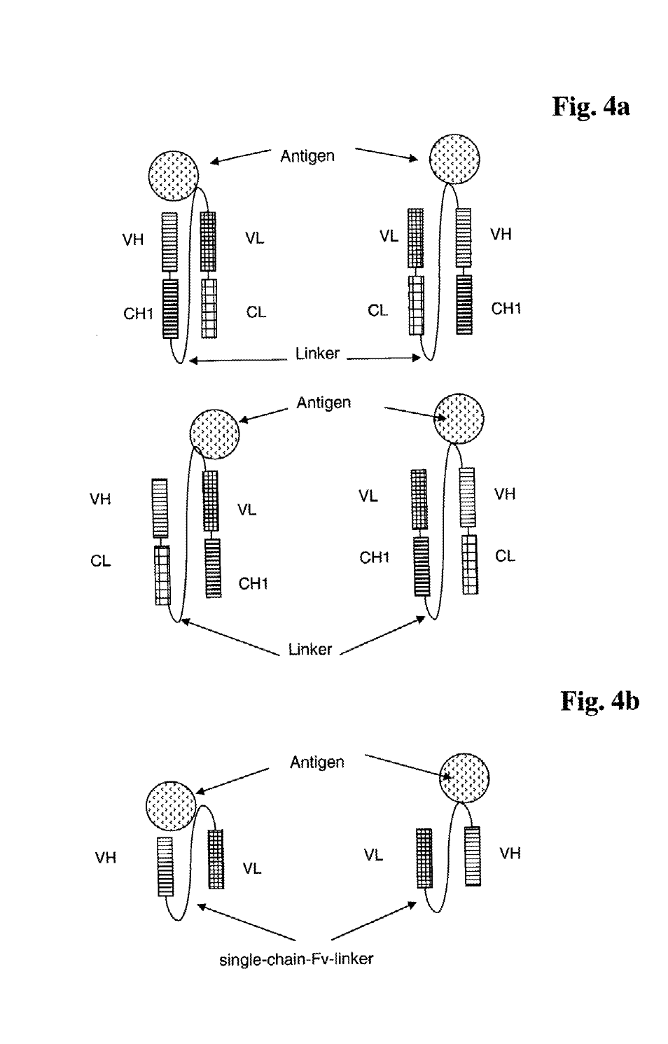

For exemplary schematic structures see FIG. 3a-c.

Accordingly the corresponding trivalent, bispecific antibody with the VHVL-Ab-nomenclature in Table 4 were expressed and purified (see in the Examples below and FIG. 3c).

Optionally the antibody heavy chain variable domain (VH) of the polypeptide under b) and the antibody light chain variable domain (VL) of the polypeptide under c) are linked and stabilized via a interchain disulfide bridge by introduction of a disulfide bond between the following positions:

i) heavy chain variable domain position 44 to light chain variable domain position 100,
ii) heavy chain variable domain position 105 to light chain variable domain position 43, or
iii) heavy chain variable domain position 101 to light chain variable domain position 100 (numbering always according to EU index of Kabat).

Techniques to introduce unnatural disulfide bridges for stabilization are described e.g. in WO 94/029350, Rajagopal, V., et al., Prot. Engin. (1997) 1453-59; Kobayashi, H., et al; Nuclear Medicine & Biology, Vol. 25, (1998) 387-393; or Schmidt, M., et al., Oncogene (1999) 18, 1711-1721. In one embodiment the optional disulfide bond between the variable domains of the polypeptides under b) and c) is between heavy chain variable domain position 44 and light chain variable domain position 100. In one embodiment the optional disulfide bond between the variable domains of the polypeptides under b) and c) is between heavy chain variable domain position 105 and light chain variable domain position 43. (numbering always according to EU index of Kabat) In one embodiment a trivalent, bispecific antibody without the optional disulfide stabilization between the variable domains VH and VL of the single chain Fab fragments is preferred.

By the fusion of a single chain Fab, Fv fragment to one of the heavy chains (FIG. 5a or 5b) or by the fusion of the different polypeptides to both heavy chains of the full lengths antibody (FIG. 3a-c) a heterodimeric, trivalent bispecific antibody results. To improve the yields of such heterodimeric trivalent, bispecific anti-ErbB-3/anti-c-Met antibodies, the CH3 domains of the full length antibody can be altered by the “knob-into-holes” technology which is described in detail with several examples in e.g. WO 96/027011, Ridgway, J. B., et al., Protein Eng 9 (1996) 617-621; and Merchant, A. M., et al., Nat Biotechnol 16 (1998) 677-681. In this method the interaction surfaces of the two CH3 domains are altered to increase the heterodimerisation of both heavy chains containing these two CH3 domains. Each of the two CH3 domains (of the two heavy chains) can be the “knob”, while the other is the “hole”. The introduction of a disulfide bridge stabilizes the heterodimers (Merchant, A. M, et al., Nature Biotech 16 (1998) 677-681; Atwell, S., et al. J. Mol. Biol. 270 (1997) 26-35) and increases the yield.

Thus in one aspect of the invention the trivalent, bispecific antibody is further is characterized in that the CH3 domain of one heavy chain of the full length antibody and the CH3 domain of the other heavy chain of the full length antibody each meet at an interface which comprises an original interface between the antibody CH3 domains;

wherein the interface is altered to promote the formation of the bivalent, bispecific antibody,
wherein the alteration is characterized in that:
a) the CH3 domain of one heavy chain is altered,
so that within the original interface the CH3 domain of one heavy chain that meets the original interface of the CH3 domain of the other heavy chain within the bivalent, bispecific antibody,
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and
b) the CH3 domain of the other heavy chain is altered,
so that within the original interface of the second CH3 domain that meets the original interface of the first CH3 domain within the trivalent, bispecific antibody
an amino acid residue is replaced with an amino acid residue having a smaller side chain volume, thereby generating a cavity within the interface of the second CH3 domain within which a protuberance within the interface of the first CH3 domain is positionable.

Preferably the amino acid residue having a larger side chain volume is selected from the group consisting of arginine (R), phenylalanine (F), tyrosine (Y), tryptophan (W).

Preferably the amino acid residue having a smaller side chain volume is selected from the group consisting of alanine (A), serine (S), threonine (T), valine (V).

In one aspect of the invention both CH3 domains are further altered by the introduction of cysteine (C) as amino acid in the corresponding positions of each CH3 domain such that a disulfide bridge between both CH3 domains can be formed.

In a preferred embodiment, the trivalent, bispecific comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M., et al., Nature Biotech 16 (1998) 677-681) e.g. by introducing a Y349C mutation into the CH3 domain of the “knobs chain” and a E356C mutation or a S354C mutation into the CH3 domain of the “hole chain”. Thus in a another preferred embodiment, the trivalent, bispecific antibody comprises Y349C, T366W mutations in one of the two CH3 domains and E356C, T366S, L368A, Y407V mutations in the other of the two CH3 domains or the trivalent, bispecific antibody comprises Y349C, T366W mutations in one of the two CH3 domains and S354C, T366S, L368A, Y407V mutations in the other of the two CH3 domains (the additional Y349C mutation in one CH3 domain and the additional E356C or S354C mutation in the other CH3 domain forming a interchain disulfide bridge) (numbering always according to EU index of Kabat). But also other knobs-in-holes technologies as described by EP 1870459A1, can be used alternatively or additionally. A preferred example for the trivalent, bispecific antibody are R409D; K370E mutations in the CH3 domain of the “knobs chain” and D399K; E357K mutations in the CH3 domain of the “hole chain” (numbering always according to EU index of Kabat).

In another preferred embodiment the trivalent, bispecific antibody comprises a T366W mutation in the CH3 domain of the “knobs chain” and T366S, L368A, Y407V mutations in the CH3 domain of the “hole chain” and additionally R409D; K370E mutations in the CH3 domain of the “knobs chain” and D399K; E357K mutations in the CH3 domain of the “hole chain”.

In another preferred embodiment the trivalent, bispecific antibody comprises Y349C, T366W mutations in one of the two CH3 domains and S354C, T366S, L368A, Y407V mutations in the other of the two CH3 domains or the trivalent, bispecific antibody comprises Y349C, T366W mutations in one of the two CH3 domains and S354C, T366S, L368A, Y407V mutations in the other of the two CH3 domains and additionally R409D; K370E mutations in the CH3 domain of the “knobs chain” and D399K; E357K mutations in the CH3 domain of the “hole chain”.

Another embodiment of the current invention is a trivalent, bispecific antibody comprising

a) a full length antibody specifically binding to human ErbB-3 and consisting of:
aa) two antibody heavy chains consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3); and
ab) two antibody light chains consisting in N-terminal to C-terminal direction of an antibody light chain variable domain (VL), and an antibody light chain constant domain (CL) (VL-CL).; and
b) one single chain Fab fragment specifically binding to human c-Met),
wherein the single chain Fab fragment consist of an antibody heavy chain variable domain (VH) and an antibody constant domain 1 (CH1), an antibody light chain variable domain (VL), an antibody light chain constant domain (CL) and a linker, and wherein the antibody domains and the linker have one of the following orders in N-terminal to C-terminal direction:
ba) VH-CH1-linker-VL-CL, or bb) VL-CL-linker-VH-CH1;
wherein the linker is a peptide of at least 30 amino acids, preferably between 32 and 50 amino acids;
and wherein the single chain Fab fragment under b) is fused to the full length antibody under a) via a peptide connector at the C- or N-terminus of the heavy or light chain (preferably at the C-terminus of the heavy chain) of the full length antibody;
wherein the peptide connector is a peptide of at least 5 amino acids, preferably between 10 and 50 amino acids.

Within this embodiment, preferably the trivalent, bispecific antibody comprises a T366W mutation in one of the two CH3 domains of and T366S, L368A, Y407V mutations in the other of the two CH3 domains and more preferably the trivalent, bispecific antibody comprises Y349C, T366W mutations in one of the two CH3 domains of and S354C (or E356C), T366S, L368A, Y407V mutations in the other of the two CH3 domains. Optionally in the embodiment the trivalent, bispecific antibody comprises R409D; K370E mutations in the CH3 domain of the “knobs chain” and D399K; E357K mutations in the CH3 domain of the “hole chain”.

Another embodiment of the current invention is a trivalent, bispecific antibody comprising

a) a full length antibody specifically binding to human ErbB-3 and consisting of:
aa) two antibody heavy chains consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3); and
ab) two antibody light chains consisting in N-terminal to C-terminal direction of an antibody light chain variable domain (VL), and an antibody light chain constant domain (CL) (VL-CL).; and
b) one single chain Fv fragment specifically binding to human c-Met),
wherein the single chain Fv fragment under b) is fused to the full length antibody under a) via a peptide connector at the C- or N-terminus of the heavy or light chain (preferably at the C-terminus of the heavy chain) of the full length antibody; and
wherein the peptide connector is a peptide of at least 5 amino acids, preferably between 10 and 50 amino acids.

Within this embodiment, preferably the trivalent, bispecific antibody comprises a T366W mutation in one of the two CH3 domains of and T366S, L368A, Y407V mutations in the other of the two CH3 domains and more preferably the trivalent, bispecific antibody comprises Y349C, T366W mutations in one of the two CH3 domains of and S354C (or E356C), T366S, L368A, Y407V mutations in the other of the two CH3 domains. Optionally in the embodiment the trivalent, bispecific antibody comprises R409D; K370E mutations in the CH3 domain of the “knobs chain” and D399K; E357K mutations in the CH3 domain of the “hole chain”.

Thus a preferred embodiment is a trivalent, bispecific antibody comprising

a) a full length antibody specifically binding to human ErbB-3 and consisting of:
aa) two antibody heavy chains consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3); and
ab) two antibody light chains consisting in N-terminal to C-terminal direction of an antibody light chain variable domain (VL), and an antibody light chain constant domain (CL) (VL-CL).; and
b) one single chain Fv fragment specifically binding to human c-Met),
wherein the single chain Fv fragment under b) is fused to the full length antibody under a) via a peptide connector at the C-terminus of the heavy chain of the full length antibody (resulting in two antibody heavy chain-single chain Fv fusion peptides); and
wherein the peptide connector is a peptide of at least 5 amino acids.

In a preferred embodiment the trivalent, bispecific antibody comprises as first antibody heavy chain-single chain Fv fusion peptide a polypeptide of SEQ ID NO:26, as second antibody heavy chain-single chain Fv fusion peptide a polypeptide of SEQ ID NO:27, and two antibody light chains of SEQ ID NO:28.

In a preferred embodiment the trivalent, bispecific antibody comprises as first antibody heavy chain-single chain Fv fusion peptide a polypeptide of SEQ ID NO:29, as second antibody heavy chain-single chain Fv fusion peptide a polypeptide of SEQ ID NO:30, and two antibody light chains of SEQ ID NO:31.

In a preferred embodiment the trivalent, bispecific antibody comprises as first antibody heavy chain-single chain Fv fusion peptide a polypeptide of SEQ ID NO:32, as second antibody heavy chain-single chain Fv fusion peptide a polypeptide of SEQ ID NO:33, and two antibody light chains of SEQ ID NO:34.

In a preferred embodiment the trivalent, bispecific antibody comprises as first antibody heavy chain-single chain Fv fusion peptide a polypeptide of SEQ ID NO:35, as second antibody heavy chain-single chain Fv fusion peptide a polypeptide of SEQ ID NO:36, and two antibody light chains of SEQ ID NO:37.

In a preferred embodiment the trivalent, bispecific antibody comprises as first antibody heavy chain-single chain Fv fusion peptide a polypeptide of SEQ ID NO:38, as second antibody heavy chain-single chain Fv fusion peptide a polypeptide of SEQ ID NO:39, and two antibody light chains of SEQ ID NO:40.

Another embodiment of the current invention is a trivalent, bispecific antibody comprising

a) a full length antibody specifically binding to human ErbB-3 and consisting of:
aa) two antibody heavy chains consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3); and
ab) two antibody light chains consisting in N-terminal to C-terminal direction of an antibody light chain variable domain (VL), and an antibody light chain constant domain (CL); and
b) a polypeptide consisting of
ba) an antibody heavy chain variable domain (VH); or
bb) an antibody heavy chain variable domain (VH) and an antibody constant domain 1 (CH1),
wherein the polypeptide is fused with the N-terminus of the VH domain via a peptide connector to the C-terminus of one of the two heavy chains of the full length antibody (resulting in an antibody heavy chain-VH fusion peptide) wherein the peptide connector is a peptide of at least 5 amino acids, preferably between 25 and 50 amino acids;
c) a polypeptide consisting of
ca) an antibody light chain variable domain (VL), or
cb) an antibody light chain variable domain (VL) and an antibody light chain constant domain (CL);
wherein the polypeptide is fused with the N-terminus of the VL domain via a peptide connector to the C-terminus of the other of the two heavy chains of the full length antibody (resulting in an antibody heavy chain—VL fusion peptide);
wherein the peptide connector is identical to the peptide connector under b);
and wherein the antibody heavy chain variable domain (VH) of the polypeptide under b) and the antibody light chain variable domain (VL) of the polypeptide under c) together form an antigen-binding site specifically binding to human c-Met.

Within this embodiment, preferably the trivalent, bispecific antibody comprises a T366W mutation in one of the two CH3 domains of and T366S, L368A, Y407V mutations in the other of the two CH3 domains and more preferably the trivalent, bispecific antibody comprises Y349C, T366W mutations in one of the two CH3 domains of and S354C (or E356C), T366S, L368A, Y407V mutations in the other of the two CH3 domains. Optionally in the embodiment the trivalent, bispecific antibody comprises R409D; K370E mutations in the CH3 domain of the “knobs chain” and D399K; E357K mutations in the CH3 domain of the “hole chain”.

In a preferred embodiment the trivalent, bispecific antibody comprises as antibody heavy chain-VH fusion peptide a polypeptide of SEQ ID NO:11, as antibody heavy chain-VL fusion peptide a polypeptide of SEQ ID NO:12, and two antibody light chains of SEQ ID NO:13.

In a preferred embodiment the trivalent, bispecific antibody comprises as antibody heavy chain-VH fusion peptide a polypeptide of SEQ ID NO:14, as antibody heavy chain-VL fusion peptide a polypeptide of SEQ ID NO:15, and two antibody light chains of SEQ ID NO:16.

In a preferred embodiment the trivalent, bispecific antibody comprises as antibody heavy chain-VH fusion peptide a polypeptide of SEQ ID NO:17, as antibody heavy chain-VL fusion peptide a polypeptide of SEQ ID NO:18, and two antibody light chains of SEQ ID NO:19.

In a preferred embodiment the trivalent, bispecific antibody comprises as antibody heavy chain-VH fusion peptide a polypeptide of SEQ ID NO:20, as antibody heavy chain-VL fusion peptide a polypeptide of SEQ ID NO:21, and two antibody light chains of SEQ ID NO:22.

In a preferred embodiment the trivalent, bispecific antibody comprises as antibody heavy chain-VH fusion peptide a polypeptide of SEQ ID NO:23, as antibody heavy chain-VL fusion peptide a polypeptide of SEQ ID NO:24, and two antibody light chains of SEQ ID NO:25.

In another aspect of the current invention the trivalent, bispecific antibody according to the invention comprises

a) a full length antibody binding to human ErbB-3 consisting of two antibody heavy chains VH-CH1-HR-CH2-CH3 and two antibody light chains VL-CL;
(wherein preferably one of the two CH3 domains comprises Y349C, T366W mutations and the other of the two CH3 domains comprises S354C (or E356C), T366S, L368A, Y407V mutations);
b) a polypeptide consisting of
ba) an antibody heavy chain variable domain (VH); or
bb) an antibody heavy chain variable domain (VH) and an antibody constant domain 1 (CH1),
wherein the polypeptide is fused with the N-terminus of the VH domain via a peptide connector to the C-terminus of one of the two heavy chains of the full length antibody
c) a polypeptide consisting of
ca) an antibody light chain variable domain (VL), or
cb) an antibody light chain variable domain (VL) and an antibody light chain constant domain (CL);
wherein the polypeptide is fused with the N-terminus of the VL domain via a peptide connector to the C-terminus of the other of the two heavy chains of the full length antibody;
and wherein the antibody heavy chain variable domain (VH) of the polypeptide under b) and the antibody light chain variable domain (VL) of the polypeptide under c) together form an antigen-binding site specifically binding to human c-Met.

### Tetravalent Bispecific Formats

In one embodiment the bispecific antibody according to the invention is tetravalent, wherein the antigen-binding site(s) that specifically bind to human c-Met, inhibit the c-Met dimerisation (as described e.g. in WO 2009/007427).

Another aspect of the current invention therefore is a tetravalent, bispecific antibody comprising

a) a full length antibody specifically binding to human ErbB-3 and consisting of two antibody heavy chains and two antibody light chains; and
b) two identical single chain Fab fragments specifically binding to human c-Met,
wherein the single chain Fab fragments under b) are fused to the full length antibody under a) via a peptide connector at the C- or N-terminus of the heavy or light chain of the full length antibody.

Another aspect of the current invention therefore is a tetravalent, bispecific antibody comprising

a) a full length antibody specifically binding to human c-Met and consisting of two antibody heavy chains and two antibody light chains; and
b) two identical single chain Fab fragments specifically binding to human ErbB-3,
wherein the single chain Fab fragments under b) are fused to the full length antibody under a) via a peptide connector at the C- or N-terminus of the heavy or light chain of the full length antibody.

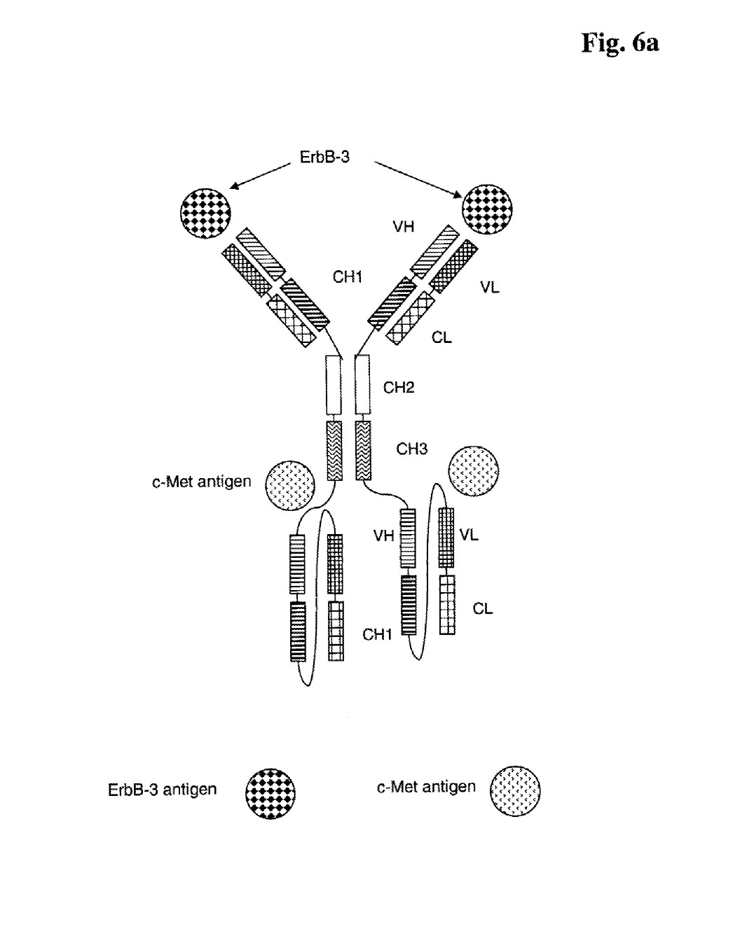

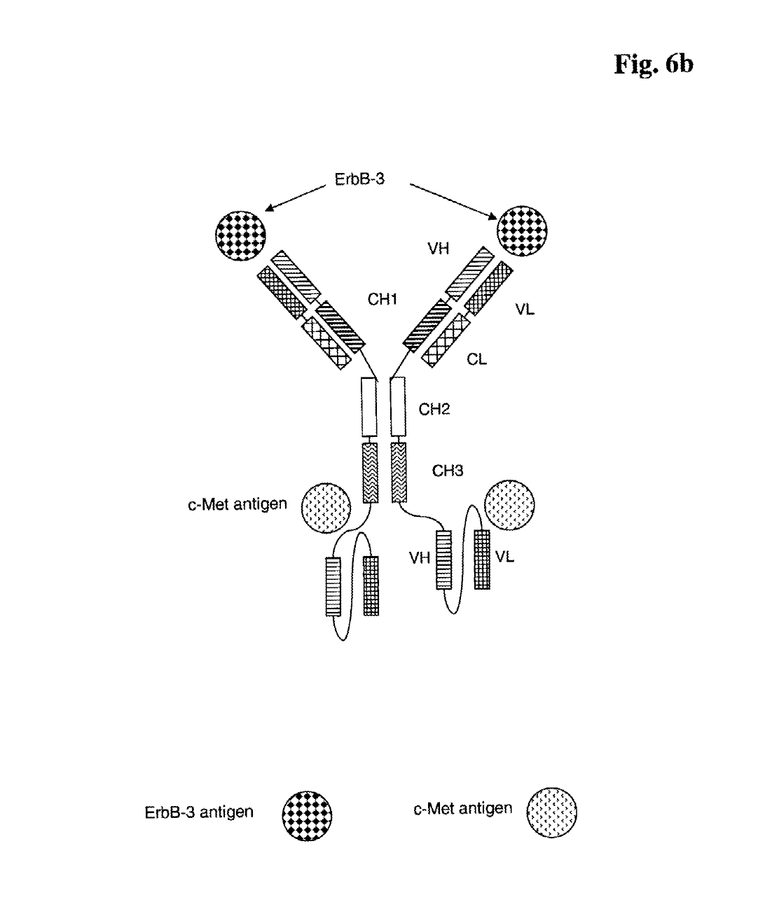

For an exemplary schematic structure see FIG. 6a.

Another aspect of the current invention therefore is a tetravalent, bispecific antibody comprising

a) a full length antibody specifically binding to human ErbB-3 and consisting of two antibody heavy chains and two antibody light chains; and
b) two identical single chain Fv fragments specifically binding to human c-Met,
wherein the single chain Fv fragments under b) are fused to the full length antibody under a) via a peptide connector at the C- or N-terminus of the heavy or light chain of the full length antibody.

Another aspect of the current invention therefore is a tetravalent, bispecific antibody comprising

a) a full length antibody specifically binding to human c-Met and consisting of two antibody heavy chains and two antibody light chains; and
b) two identical single chain Fv fragments specifically binding to human ErbB-3,
wherein the single chain Fv fragments under b) are fused to the full length antibody under a) via a peptide connector at the C- or N-terminus of the heavy or light chain of the full length antibody.

For an exemplary schematic structure see FIG. 6b.

In one preferred embodiment the single chain Fab or Fv fragments binding human c-Met or human ErbB-3 are fused to the full length antibody via a peptide connector at the C-terminus of the heavy chains of the full length antibody.

Another embodiment of the current invention is a tetravalent, bispecific antibody comprising

a) a full length antibody specifically binding to human ErbB-3 and consisting of:
aa) two identical antibody heavy chains consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3); and
ab) two identical antibody light chains consisting in N-terminal to C-terminal direction of an antibody light chain variable domain (VL), and an antibody light chain constant domain (CL) (VL-CL).; and
b) two single chain Fab fragments specifically binding to human c-Met,
wherein the single chain Fab fragments consist of an antibody heavy chain variable domain (VH) and an antibody constant domain 1 (CH1), an antibody light chain variable domain (VL), an antibody light chain constant domain (CL) and a linker, and wherein the antibody domains and the linker have one of the following orders in N-terminal to C-terminal direction: ba) VH-CH1-linker-VL-CL, or bb) VL-CL-linker-VH-CH1;
wherein the linker is a peptide of at least 30 amino acids, preferably between 32 and 50 amino acids;
and wherein the single chain Fab fragments under b) are fused to the full length antibody under a) via a peptide connector at the C- or N-terminus of the heavy or light chain of the full length antibody;
wherein the peptide connector is a peptide of at least 5 amino acids, preferably between 10 and 50 amino acids.

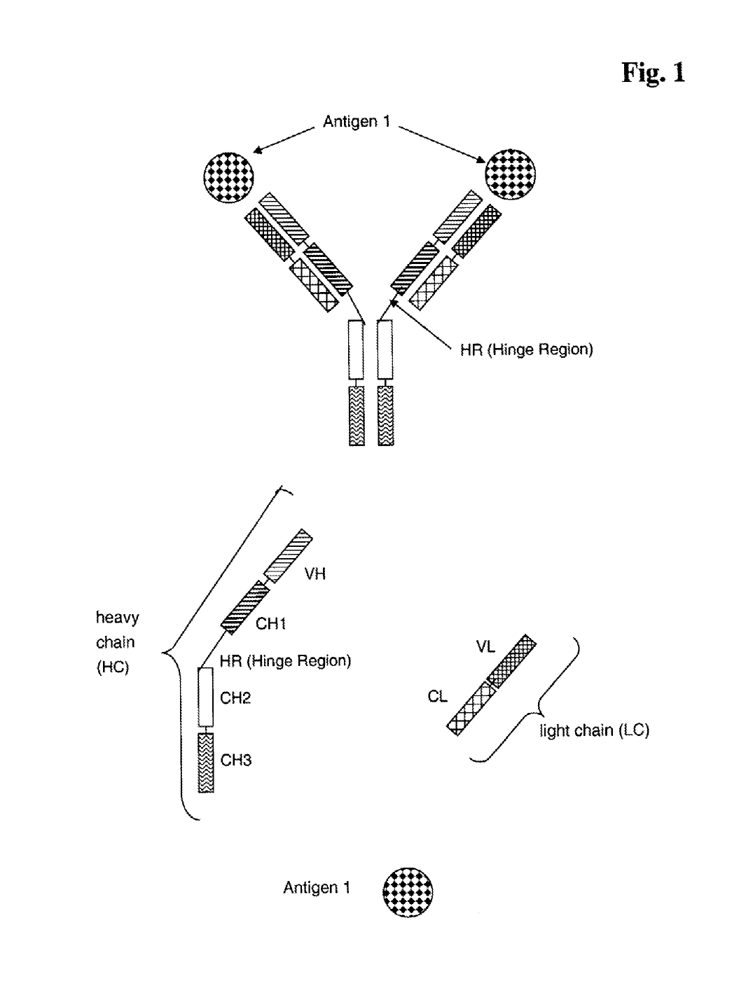

The term “full length antibody” as used either in the trivalent or tetravalent format denotes an antibody consisting of two “full length antibody heavy chains” and two “full length antibody light chains” (see FIG. 1). A “full length antibody heavy chain” is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. A “full length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be κ (kappa) or λ(lambda). The two full length antibody chains are linked together via inter-polypeptide disulfide bonds between the CL domain and the CH1 domain and between the hinge regions of the full length antibody heavy chains. Examples of typical full length antibodies are natural antibodies like IgG (e.g. IgG 1 and IgG2), IgM, IgA, IgD, and IgE. The full length antibodies according to the invention can be from a single species e.g. human, or they can be chimerized or humanized antibodies. The full length antibodies according to the invention comprise two antigen binding sites each formed by a pair of VH and VL, which both specifically bind to the same antigen. The C-terminus of the heavy or light chain of the full length antibody denotes the last amino acid at the C-terminus of the heavy or light chain. The N-terminus of the heavy or light chain of the full length antibody denotes the last amino acid at the N-terminus of the heavy or light chain.

The term “peptide connector” as used within the invention denotes a peptide with amino acid sequences, which is preferably of synthetic origin. These peptide connectors according to invention are used to fuse the single chain Fab fragments to the C- or N-terminus of the full length antibody to form a multispecific antibody according to the invention. Preferably the peptide connectors under b) are peptides with an amino acid sequence with a length of at least 5 amino acids, preferably with a length of 5 to 100, more preferably of 10 to 50 amino acids In one embodiment the peptide connector is (G×S)n or (G×S)nGm with G=glycine,

S=serine, and (x=3, n=3, 4, 5 or 6, and m=0, 1, 2 or 3) or (x=4,n=2, 3, 4 or 5 and m=0, 1, 2 or 3), preferably x=4 and n=2 or 3, more preferably with x=4, n=2. Preferably in the trivalent, bispecific antibodies wherein a VH or a VH-CH1 polypeptide and a VL or a VL-C L polypeptide (FIG. 7a-c) are fused via two identical peptide connectors to the C-terminus of a full length antibody, the peptide connectors are peptides of at least 25 amino acids, preferably peptides between 30 and 50 amino acids and more preferably the peptide connector is (G×S)n or (G×S)nGm with G=glycine, S=serine, and (x=3, n=6, 7 or 8, and m=0, 1, 2 or 3) or (x=4,n=5, 6, or 7 and m=0, 1, 2 or 3), preferably x=4 and n=5, 6, 7.

A “single chain Fab fragment” (see FIG. 2a) is a polypeptide consisting of an antibody heavy chain variable domain (VH), an antibody constant domain 1 (CH1), an antibody light chain variable domain (VL), an antibody light chain constant domain (CL) and a linker, wherein the antibody domains and the linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein the linker is a polypeptide of at least 30 amino acids, preferably between 32 and 50 amino acids. The single chain Fab fragments a) VH-CH1-linker-VL-CL, b) VL-CL-linker-VH-CH1, c) VH-CL-linker-VL-CH1 and d) VL-CH1-linker-VH-CL, are stabilized via the natural disulfide bond between the CL domain and the CH1 domain. The term “N-terminus denotes the last amino acid of the N-terminus, The term “C-terminus denotes the last amino acid of the C-terminus.

The term “linker” is used within the invention in connection with single chain Fab fragments and denotes a peptide with amino acid sequences, which is preferably of synthetic origin. These peptides according to invention are used to link a) VH-CH1 to VL-CL, b) VL-CL to VH-CH1, c) VH-CL to VL-CH1 or d) VL-CH1 to VH-CL to form the following single chain Fab fragments according to the invention a) VH-CH1-linker-VL-CL, b) VL-CL-linker-VH-CH1, c) VH-CL-linker-VL-CH1 or d) VL-CH1-linker-VH-CL. The linker within the single chain Fab fragments is a peptide with an amino acid sequence with a length of at least 30 amino acids, preferably with a length of 32 to 50 amino acids. In one embodiment the linker is (G×S)n with G=glycine, S=serine, (x=3, n=8, 9 or 10 and m=0, 1, 2 or 3) or (x=4 and n=6, 7 or 8 and m=0, 1, 2 or 3), preferably with x=4, n=6 or 7 and m=0, 1, 2 or 3, more preferably with x=4, n=7 and m=2. In one embodiment the linker is (G₄S)₆G₂.

In a preferred embodiment the antibody domains and the linker in the single chain Fab fragment have one of the following orders in N-terminal to C-terminal direction:

a) VH-CH1-linker-VL-CL, or b) VL-CL-linker-VH-CH1, more preferably VL-CL-linker-VH-CH1.

In another preferred embodiment the antibody domains and the linker in the single chain Fab fragment have one of the following orders in N-terminal to C-terminal direction:

a) VH-CL-linker-VL-CH1 or b) VL-CH1-linker-VH-CL.

Optionally in the single chain Fab fragment, additionally to the natural disulfide bond between the CL-domain and the CH1 domain, also the antibody heavy chain variable domain (VH) and the antibody light chain variable domain (VL) are disulfide stabilized by introduction of a disulfide bond between the following positions:

i) heavy chain variable domain position 44 to light chain variable domain position 100,
ii) heavy chain variable domain position 105 to light chain variable domain position 43, or
iii) heavy chain variable domain position 101 to light chain variable domain position 100 (numbering always according to EU index of Kabat).

Such further disulfide stabilization of single chain Fab fragments is achieved by the introduction of a disulfide bond between the variable domains VH and VL of the single chain Fab fragments. Techniques to introduce unnatural disulfide bridges for stabilization for a single chain Fv are described e.g. in WO 94/029350, Rajagopal, V., et al, Prot. Engin. (1997) 1453-59; Kobayashi, H., et al., Nuclear Medicine & Biology, Vol. 25, (1998) 387-393; or Schmidt, M., et al., Oncogene (1999) 18, 1711-1721. In one embodiment the optional disulfide bond between the variable domains of the single chain Fab fragments comprised in the antibody according to the invention is between heavy chain variable domain position 44 and light chain variable domain position 100. In one embodiment the optional disulfide bond between the variable domains of the single chain Fab fragments comprised in the antibody according to the invention is between heavy chain variable domain position 105 and light chain variable domain position 43 (numbering always according to EU index of Kabat).

In an embodiment single chain Fab fragment without the optional disulfide stabilization between the variable domains VH and VL of the single chain Fab fragments are preferred.

A “single chain Fv fragment” (see FIG. 2b) is a polypeptide consisting of an antibody heavy chain variable domain (VH), an antibody light chain variable domain (VL), and a single-chain-Fv-linker, wherein the antibody domains and the single-chain-Fv-linker have one of the following orders in N-terminal to C-terminal direction: a) VH-single-chain-Fv-linker-VL, b) VL-single-chain-Fv-linker-VH; preferably a) VH-single-chain-Fv-linker-VL, and wherein the single-chain-Fv-linker is a polypeptide of with an amino acid sequence with a length of at least 15 amino acids, in one embodiment with a length of at least 20 amino acids. The term “N-terminus denotes the last amino acid of the N-terminus, The term “C-terminus denotes the last amino acid of the C-terminus.

The term “single-chain-Fv-linker” as used within single chain Fv fragment denotes a peptide with amino acid sequences, which is preferably of synthetic origin. The single-chain-Fv-linker is a peptide with an amino acid sequence with a length of at least 15 amino acids, in one embodiment with a length of at least 20 amino acids and preferably with a length between 15 and 30 amino acids. In one embodiment the single-chain-linker is (G×S)n with G=glycine, S=serine, (x=3 and n=4, 5 or 6) or (x=4 and n=3, 4, 5 or 6), preferably with x=4, n=3, 4 or 5, more preferably with x=4, n=3 or 4. In one embodiment the ingle-chain-Fv-linker is (G₄S)₃ or (G₄S)₄.

Furthermore the single chain Fv fragments are preferably disulfide stabilized. Such further disulfide stabilization of single chain antibodies is achieved by the introduction of a disulfide bond between the variable domains of the single chain antibodies and is described e.g. in WO 94/029350, Rajagopal, V., et al., Prot. Engin. 10 (1997) 1453-59; Kobayashi, H., et al., Nuclear Medicine & Biology, Vol. 25 (1998) 387-393; or Schmidt, M., et al, Oncogene 18 (1999) 1711-1721.

In one embodiment of the disulfide stabilized single chain Fv fragments, the disulfide bond between the variable domains of the single chain Fv fragments comprised in the antibody according to the invention is independently for each single chain Fv fragment selected from:
i) heavy chain variable domain position 44 to light chain variable domain position 100,
ii) heavy chain variable domain position 105 to light chain variable domain position 43, or
iii) heavy chain variable domain position 101 to light chain variable domain position 100.

In one embodiment the disulfide bond between the variable domains of the single chain Fv fragments comprised in the antibody according to the invention is between heavy chain variable domain position 44 and light chain variable domain position 100.

The antibody according to the invention is produced by recombinant means. Thus, one aspect of the current invention is a nucleic acid encoding the antibody according to the invention and a further aspect is a cell comprising the nucleic acid encoding an antibody according to the invention. Methods for recombinant production are widely known in the state of the art and comprise protein expression in prokaryotic and eukaryotic cells with subsequent isolation of the antibody and usually purification to a pharmaceutically acceptable purity. For the expression of the antibodies as aforementioned in a host cell, nucleic acids encoding the respective modified light and heavy chains are inserted into expression vectors by standard methods. Expression is performed in appropriate prokaryotic or eukaryotic host cells like CHO cells, NS0 cells, SP2/0 cells, HEK293 cells, COS cells, PER.C6 cells, yeast, or E. coli cells, and the antibody is recovered from the cells (supernatant or cells after lysis). General methods for recombinant production of antibodies are well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

The bispecific antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies is readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant monoclonal antibodies in the host cells.

Amino acid sequence variants (or mutants) of the bispecific antibody are prepared by introducing appropriate nucleotide changes into the antibody DNA, or by nucleotide synthesis. Such modifications can be performed, however, only in a very limited range, e.g. as described above. For example, the modifications do not alter the above mentioned antibody characteristics such as the IgG isotype and antigen binding, but may improve the yield of the recombinant production, protein stability or facilitate the purification.

The term “host cell” as used in the current application denotes any kind of cellular system which can be engineered to generate the antibodies according to the current invention. In one embodiment HEK293 cells and CHO cells are used as host cells. As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included.

Expression in NS0 cells is described by, e.g., Barnes, L. M., et al., Cytotechnology 32 (2000) 109-123; Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; and Norderhaug, L., et al., J. Immunol. Methods 204 (1997) 77-87. A preferred transient expression system (HEK 293) is described by Schlaeger, E.-J., and Christensen, K., in Cytotechnology 30 (1999) 71-83 and by Schlaeger, E.-J., in J. Immunol. Methods 194 (1996) 191-199.

The control sequences that are suitable for prokaryotes, for example, include a promoter, optionally an operator sequence, and a ribosome binding site. Eukaryotic cells are known to utilize promoters, enhancers and polyadenylation signals.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

Purification of antibodies is performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987). Different methods are well established and widespread used for protein purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A., Appl. Biochem. Biotech. 75 (1998) 93-102).

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

The term “transformation” as used herein refers to process of transfer of a vectors/nucleic acid into a host cell. If cells without formidable cell wall barriers are used as host cells, transfection is carried out e.g. by the calcium phosphate precipitation method as described by Graham, F. L., and van der Eh, A. J., Virology 52 (1973) 456-467. However, other methods for introducing DNA into cells such as by nuclear injection or by protoplast fusion may also be used. If prokaryotic cells or cells which contain substantial cell wall constructions are used, e.g. one method of transfection is calcium treatment using calcium chloride as described by Cohen, F. N., et al, PNAS. 69 (1972) 7110ff.

As used herein, “expression” refers to the process by which a nucleic acid is transcribed into mRNA and/or to the process by which the transcribed mRNA (also referred to as transcript) is subsequently being translated into peptides, polypeptides, or proteins. The transcripts and the encoded polypeptides are collectively referred to as gene product. If the polynucleotide is derived from genomic DNA, expression in a eukaryotic cell may include splicing of the mRNA.

A “vector” is a nucleic acid molecule, in particular self-replicating, which transfers an inserted nucleic acid molecule into and/or between host cells. The term includes vectors that function primarily for insertion of DNA or RNA into a cell (e.g., chromosomal integration), replication of vectors that function primarily for the replication of DNA or RNA, and expression vectors that function for transcription and/or translation of the DNA or RNA. Also included are vectors that provide more than one of the functions as described.

An “expression vector” is a polynucleotide which, when introduced into an appropriate host cell, can be transcribed and translated into a polypeptide. An “expression system” usually refers to a suitable host cell comprised of an expression vector that can function to yield a desired expression product.

### Pharmaceutical Composition

One aspect of the invention is a pharmaceutical composition comprising an antibody according to the invention. Another aspect of the invention is the use of an antibody according to the invention for the manufacture of a pharmaceutical composition. A further aspect of the invention is a method for the manufacture of a pharmaceutical composition comprising an antibody according to the invention. In another aspect, the present invention provides a composition, e.g. a pharmaceutical composition, containing an antibody according to the present invention, formulated together with a pharmaceutical carrier.

One embodiment of the invention is the bispecific antibody according to the invention for the treatment of cancer.

Another aspect of the invention is a pharmaceutical composition for the treatment of cancer.

Another aspect of the invention is the use of an antibody according to the invention for the manufacture of a medicament for the treatment of cancer.

Another aspect of the invention is method of treatment of patient suffering from cancer by administering an antibody according to the invention to a patient in the need of such treatment.

As used herein, “pharmaceutical carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion).

A composition of the present invention can be administered by a variety of methods known in the art. As will be appreciated by the skilled artisan, the route and/or mode of administration will vary depending upon the desired results. To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion.

The term cancer as used herein refers to proliferative diseases, such as lymphomas, lymphocytic leukemias, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

Another aspect of the invention is the bispecific antibody according to the invention or the pharmaceutical composition as anti-angiogenic agent. Such anti-angiogenic agent can be used for the treatment of cancer, especially solid tumors, and other vascular diseases.

One embodiment of the invention is the bispecific antibody according to the invention for the treatment of vascular diseases.

Another aspect of the invention is the use of an antibody according to the invention for the manufacture of a medicament for the treatment of vascular diseases.

Another aspect of the invention is method of treatment of patient suffering from vascular diseases by administering an antibody according to the invention to a patient in the need of such treatment.

The term “vascular diseases” includes Cancer, Inflammatory diseases, Atherosclerosis, Ischemia, Trauma, Sepsis, COPD, Asthma, Diabetes, AMD, Retinopathy, Stroke, Adipositas, Acute lung injury, Hemorrhage, Vascular leak e.g. Cytokine induced, Allergy, Graves' Disease, Hashimoto's Autoimmune Thyroiditis, Idiopathic Thrombocytopenic Purpura, Giant Cell Arteritis, Rheumatoid Arthritis, Systemic Lupus Erythematosus (SLE), Lupus Nephritis, Crohn's Disease, Multiple Sclerosis, Ulcerative Colitis, especially to solid tumors, intraocular neovascular syndromes such as proliferative retinopathies or age-related macular degeneration (AMD), rheumatoid arthritis, and psoriasis (Folkman, J., and Shing, Y., et al., J. Biol. Chem. 267 (1992) 10931-10934; Klagsbrun, M., et al., Annu Rev. Physiol. 53 (1991) 217-239; and Garner, A., Vascular diseases, In: Pathobiology of ocular disease, A dynamic approach, Garner, A., and Klintworth, G. K., (eds.), 2nd edition, Marcel Dekker, New York (1994), pp 1625-1710).

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The composition must be sterile and fluid to the extent that the composition is deliverable by syringe. In addition to water, the carrier preferably is an isotonic buffered saline solution.

Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

It has now been found that the bispecific antibodies against human ErbB-3 and human c-Met according to the current invention have valuable characteristics such as biological or pharmacological activity, pharmacokinetic properties. The bispecific <ErbB3-c-Met> antibodies according to the invention show reduced internalization compared to their parent <ErbB3> and/or <c-Met> antibodies.

The following examples, sequence listing and figures are provided to aid the understanding of the present invention, the true scope of which is set forth in the appended claims. It is understood that modifications can be made in the procedures set forth without departing from the spirit of the invention.

### Description of the Amino Acid Sequences

SEQ ID NO:1 heavy chain variable domain <ErbB3> HER3 clone 29
SEQ ID NO:2 light chain variable domain <ErbB3> HER3 clone 29
SEQ ID NO:3 heavy chain variable domain <c-Met> Mab 5D5
SEQ ID NO:4 light chain variable domain <c-Met> Mab 5D5
SEQ ID NO:5 heavy chain <ErbB3> HER3 clone 29
SEQ ID NO:6 light chain <ErbB3> HER3 clone 29
SEQ ID NO:7 heavy chain <c-Met> Mab 5D5
SEQ ID NO:8 light chain <c-Met> Mab 5D5
SEQ ID NO:9 heavy chain <c-Met> Fab 5D5
SEQ ID NO:10 light chain <c-Met> Fab 5D5
SEQ ID NO:11 heavy chain 1 <ErbB3-c-Met> Her3/Met_KHSS
SEQ ID NO:12 heavy chain 2 <ErbB3-c-Met> Her3/Met)KHSS
SEQ ID NO:13 light chain <ErbB3-c-Met> Her3/Met_KHSS
SEQ ID NO:14 heavy chain 1 <ErbB3-c-Met> Her3/Met_SSKH
SEQ ID NO:15 heavy chain 2 <ErbB3-c-Met> Her3/Met_SSKH
SEQ ID NO:16 light chain <ErbB3-c-Met> Her3/Met_SSKH
SEQ ID NO:17 heavy chain 1 <ErbB3-c-Met> Her3/Met_SSKHSS
SEQ ID NO:18 heavy chain 2 <ErbB3-c-Met> Her3/Met_SSKHSS
SEQ ID NO:19 light chain <ErbB3-c-Met> Her3/Met_SSKHSS
SEQ ID NO:20 heavy chain 1 <ErbB3-c-Met> Her3/Met_{—}1C
SEQ ID NO:21 heavy chain 2 <ErbB3-c-Met> Her3/Met_{—}1C
SEQ ID NO:22 light chain <ErbB3-c-Met> Her3/Met_{—}1C
SEQ ID NO:23 heavy chain 1 <ErbB3-c-Met> Her3/Met_{—}6C
SEQ ID NO:24 heavy chain 2 <ErbB3-c-Met> Her3/Met_{—}6C
SEQ ID NO:25 light chain <ErbB3-c-Met> Her3/Met_{—}6C
SEQ ID NO:26 heavy chain 1 <ErbB3-c-Met> Her3/Met_scFvSSKHSS
SEQ ID NO:27 heavy chain 2 <ErbB3-c-Met> Her3/Met_scFvSSKHSS
SEQ ID NO:28 light chain <ErbB3-c-Met> Her3/Met_scFvSSKHSS
SEQ ID NO:29 heavy chain 1 <ErbB3-c-Met> Her3/Me_scFvSSKH
SEQ ID NO:30 heavy chain 2 <ErbB3-c-Met> Her3/Me_scFvSSKH
SEQ ID NO:31 light chain <ErbB3-c-Met> Her3/Me_scFvSSKH
SEQ ID NO:32 heavy chain 1 <ErbB3-c-Met> Her3/Me_scFvKH
SEQ ID NO:33 heavy chain 2 <ErbB3-c-Met> Her3/Me_scFvKH
SEQ ID NO:34 light chain <ErbB3-c-Met> Her3/Me_scFvKH
SEQ ID NO:35 heavy chain 1 <ErbB3-c-Met> Her3/Me_scFvKHSB
SEQ ID NO:36 heavy chain 2 <ErbB3-c-Met> Her3/Me_scFvKHSB
SEQ ID NO:37 light chain <ErbB3-c-Met> Her3/Me_scFvKHSB
SEQ ID NO:38 heavy chain 1 <ErbB3-c-Met> Her3/Met_scFvKHSBSS
SEQ ID NO:39 heavy chain 2 <ErbB3-c-Met> Her3/Met_scFvKHSBSS
SEQ ID NO:40 light chain <ErbB3-c-Met> Her3/Met_scFvKHSBSS
SEQ ID NO:41 heavy chain constant region of human IgG1
SEQ ID NO:42 heavy chain constant region of human IgG3
SEQ ID NO:43 human light chain kappa constant region
SEQ ID NO:44 human light chain lambda constant region
SEQ ID NO:45 human c-Met
SEQ ID NO:46 human ErbB-3
SEQ ID NO:47 heavy chain variable domain VH, <ErbB3> Mab 205 (murine)
SEQ ID NO:48 light chain variable domain VL, <ErbB3> Mab 205 (murine)
SEQ ID NO:49 heavy chain variable domain VH, <ErbB3> Mab 205.10 (humanized)
SEQ ID NO:50 light chain variable domain VL, <ErbB3> Mab 205.10.1 (humanized)
SEQ ID NO:51 light chain variable domain VL, <ErbB3> Mab 205.10.2 (humanized)
SEQ ID NO:52 light chain variable domain VL, <ErbB3> Mab 205.10.3 (humanized)
SEQ ID NO:53 heavy chain CDR3H, <ErbB3> Mab 205.10
SEQ ID NO:54 heavy chain CDR2H, <ErbB3> Mab 205.10
SEQ ID NO:55 heavy chain CDR1H, <ErbB3> Mab 205.10
SEQ ID NO:56 light chain CDR3L, <ErbB3> Mab 205.10
SEQ ID NO:57 light chain CDR2L, <ErbB3> Mab 205.10
SEQ ID NO:58 light chain CDR1L (variant 1), <ErbB3> Mab 205.10
SEQ ID NO:59 light chain CDR1L (variant 2), <ErbB3> Mab 205.10
SEQ ID NO:60 heavy chain CDR3H, <ErbB3> HER3 clone 29
SEQ ID NO: 61 heavy chain CDR2H, <ErbB3> HER3 clone 29
SEQ ID NO: 62 heavy chain CDR1H, <ErbB3> HER3 clone 29
SEQ ID NO: 63 light chain CDR3L, <ErbB3> HER3 clone 29
SEQ ID NO: 64 light chain CDR2L, <ErbB3> HER3 clone 29
SEQ ID NO: 65 light chain CDR1L<ErbB3> HER3 clone 29
SEQ ID NO: 66 heavy chain CDR3H, <c-Met> Mab 5D5
SEQ ID NO: 67 heavy chain CDR2H, <c-Met> Mab 5D5
SEQ ID NO: 68 heavy chain CDR1H, <c-Met> Mab 5D5
SEQ ID NO: 69 light chain CDR3L, <c-Met> Mab 5D5
SEQ ID NO: 70 light chain CDR2L, <c-Met> Mab 5D5
SEQ ID NO: 71 light chain CDR1L<c-Met> Mab 5D5

### EXPERIMENTAL PROCEDURE

### Examples

### Materials & Methods

### Recombinant DNA Techniques

Standard methods were used to manipulate DNA as described in Sambrook, J. et al., Molecular cloning: A laboratory manual; Cold Spring Harbor Laboratory Press, Cold Spring Harbor, N.Y., 1989. The molecular biological reagents were used according to the manufacturer's instructions.

### DNA and Protein Sequence Analysis and Sequence Data Management

General information regarding the nucleotide sequences of human immunoglobulins light and heavy chains is given in: Kabat, E. A. et al., (1991) Sequences of Proteins of Immunological Interest, Fifth Ed., NIH Publication No 91-3242. Amino acids of antibody chains are numbered according to EU numbering (Edelman, G. M., et al., PNAS 63 (1969) 78-85; Kabat, E. A., et al., (1991) Sequences of Proteins of Immunological Interest, Fifth Ed., NIH Publication No 91-3242). The GCG's (Genetics Computer Group, Madison, Wis.) software package version 10.2 and Infomax's Vector NTI Advance suite version 8.0 was used for sequence creation, mapping, analysis, annotation and illustration.

### DNA Sequencing

DNA sequences were determined by double strand sequencing performed at SequiServe (Vaterstetten, Germany) and Geneart AG (Regensburg, Germany).

### Gene Synthesis

Desired gene segments were prepared by Geneart AG (Regensburg, Germany) from synthetic oligonucleotides and PCR products by automated gene synthesis. The gene segments which are flanked by singular restriction endonuclease cleavage sites were cloned into pGA18 (ampR) plasmids. The plasmid DNA was purified from transformed bacteria and concentration determined by UV spectroscopy. The DNA sequence of the subcloned gene fragments was confirmed by DNA sequencing. Gene Segments coding “knobs-into-hole” Her3 (clone 29), antibody heavy chain carrying a T366W mutation in the CH3 domain with a C-terminal 5D5 VH region linked by a (G₄S)ₙ peptide connector as well as “knobs-into-hole” Her3 (clone 29) antibody heavy chain carrying T366S, L368A and Y407V mutations with a C-terminal 5D5 VL region linked by a (G₄S)ₙ peptide connector were synthesized with 5′-BamHI and 3′-XbaI restriction sites. In a similar manner, DNA sequences coding “knobs-into-hole” Her3 (clone 29) antibody heavy chain carrying S354C and T366W mutations in the CH3 domain with a C-terminal 5D5 VH region linked by a (G₄S)ₙ peptide connector as well as “knobs-into-hole” Her3 (clone 29) antibody heavy chain carrying Y349C, T366S, L368A and Y407V mutations with a C-terminal 5D5 VL region linked by a (G₄S)ₙ peptide connector were prepared by gene synthesis with flanking BamHI and XbaI restriction sites. Finally, DNA sequences encoding unmodified heavy and light chains of the Her3 (clone 29) and 5D5 antibody were synthesized with flanking BamHI and XbaI restriction sites. All constructs were designed with a 5′-end DNA sequence coding for a leader peptide (MGWSCIILFLVATATGVHS), which targets proteins for secretion in eukaryotic cells. Gene synthesis for other bispecific antibodies described below, was performed analogously using the respective element of the variable and constant region (e.g. specified in the design section below and Tables 1 to 5).

### Construction of the Expression Plasmids

A Roche expression vector was used for the construction of all heavy and light chain scFv fusion protein encoding expression plasmids. The vector is composed of the following elements:
a hygromycin resistance gene as a selection marker,
an origin of replication, oriP, of Epstein-Barr virus (EBV),
an origin of replication from the vector pUC18 which allows replication of this plasmid in E. coli
a beta-lactamase gene which confers ampicillin resistance in E. coli,
the immediate early enhancer and promoter from the human cytomegalovirus (HCMV),
the human 1-immunoglobulin polyadenylation (“poly A”) signal sequence, and
unique BamHI and XbaI restriction sites.

The immunoglobulin fusion genes comprising the heavy or light chain constructs as well as “knobs-into-hole” constructs with C-terminal VH and VL domains were prepared by gene synthesis and cloned into pGA18 (ampR) plasmids as described. The pG18 (ampR) plasmids carrying the synthesized DNA segments and the Roche expression vector were digested with BamHI and XbaI restriction enzymes (Roche Molecular Biochemicals) and subjected to agarose gel electrophoresis. Purified heavy and light chain coding DNA segments were then ligated to the isolated Roche expression vector BamHI/XbaI fragment resulting in the final expression vectors. The final expression vectors were transformed into E. coli cells, expression plasmid DNA was isolated (Miniprep) and subjected to restriction enzyme analysis and DNA sequencing. Correct clones were grown in 150 ml LB-Amp medium, again plasmid DNA was isolated (Maxiprep) and sequence integrity confirmed by DNA sequencing.

### Transient Expression of Immunoglobulin Variants in HEK293 Cells

Recombinant immunoglobulin variants were expressed by transient transfection of human embryonic kidney 293-F cells using the FreeStyle™ 293 Expression System according to the manufacturer's instruction (Invitrogen, USA). Briefly, suspension FreeStyle™ 293-F cells were cultivated in FreeStyle™ 293 Expression medium at 37° C./8% CO₂ and the cells were seeded in fresh medium at a density of 1−2×10⁶ viable cells/ml on the day of transfection. DNA-293Fectin™ complexes were prepared in Opti-MEM® I medium (Invitrogen, USA) using 325 μl of 293Fectin™ (Invitrogen, Germany) and 250 μg of heavy and light chain plasmid DNA in a 1:1 molar ratio for a 250 ml final transfection volume. “Knobs-into-hole” DNA-293fectin complexes were prepared in Opti-MEM® I medium (Invitrogen, USA) using 325 μl of 293Fectin™ (Invitrogen, Germany) and 250 μg of “Knobs-into-hole” heavy chain 1 and 2 and light chain plasmid DNA in a 1:1:2 molar ratio for a 250 ml final transfection volume. Antibody containing cell culture supernatants were harvested 7 days after transfection by centrifugation at 14000 g for 30 minutes and filtered through a sterile filter (0.22 μm). Supernatants were stored at −20° C. until purification.

### Purification of Bispecific and Control Antibodies

Bispecific and control antibodies were purified from cell culture supernatants by affinity chromatography using Protein A-Sepharose™ (GE Healthcare, Sweden) and Superdex200 size exclusion chromatography. Briefly, sterile filtered cell culture supernatants were applied on a HiTrap ProteinA HP (5 ml) column equilibrated with PBS buffer (10 mM Na₂HPO₄, 1 mM KH₂PO₄, 137 mM NaCl and 2.7 mM KCl, pH 7.4). Unbound proteins were washed out with equilibration buffer. Antibody and antibody variants were eluted with 0.1 M citrate buffer, pH 2.8, and the protein containing fractions were neutralized with 0.1 ml 1 M Tris, pH 8.5. Then, the eluted protein fractions were pooled, concentrated with an Amicon Ultra centrifugal filter device (MWCO: 30 K, Millipore) to a volume of 3 ml and loaded on a Superdex200 HiLoad 120 ml 16/60 gel filtration column (GE Healthcare, Sweden) equilibrated with 20 mM Histidin, 140 mM NaCl, pH 6.0. Fractions containing purified bispecific and control antibodies with less than 5% high molecular weight aggregates were pooled and stored as 1.0 mg/ml aliquots at −80° C. Fab fragments were generated by a Papain digest of the purified 5D5 monoclonal antibody and subsequent removal of contaminating Fc domains by Protein A chromatography. Unbound Fab fragments were further purified on a Superdex200 HiLoad 120 ml 16/60 gel filtration column (GE Healthcare, Sweden) equilibrated with 20 mM Histidin, 140 mM NaCl, pH 6.0, pooled and stored as 1.0 mg/ml aliquots at −80° C.

### Analysis of Purified Proteins

The protein concentration of purified protein samples was determined by measuring the optical density (OD) at 280 nm, using the molar extinction coefficient calculated on the basis of the amino acid sequence. Purity and molecular weight of bispecific and control antibodies were analyzed by SDS-PAGE in the presence and absence of a reducing agent (5 mM

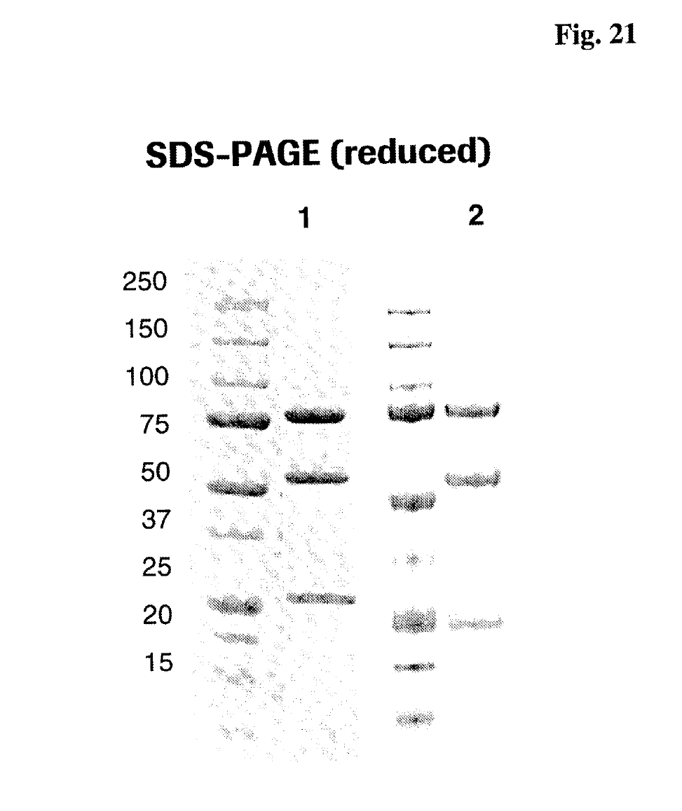

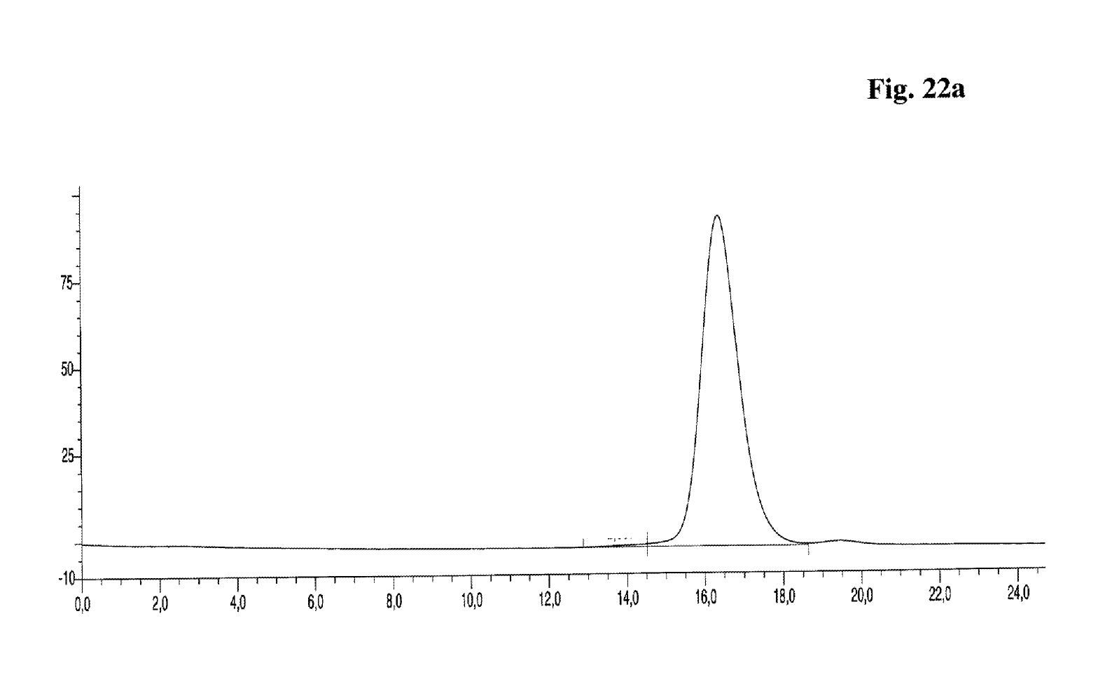

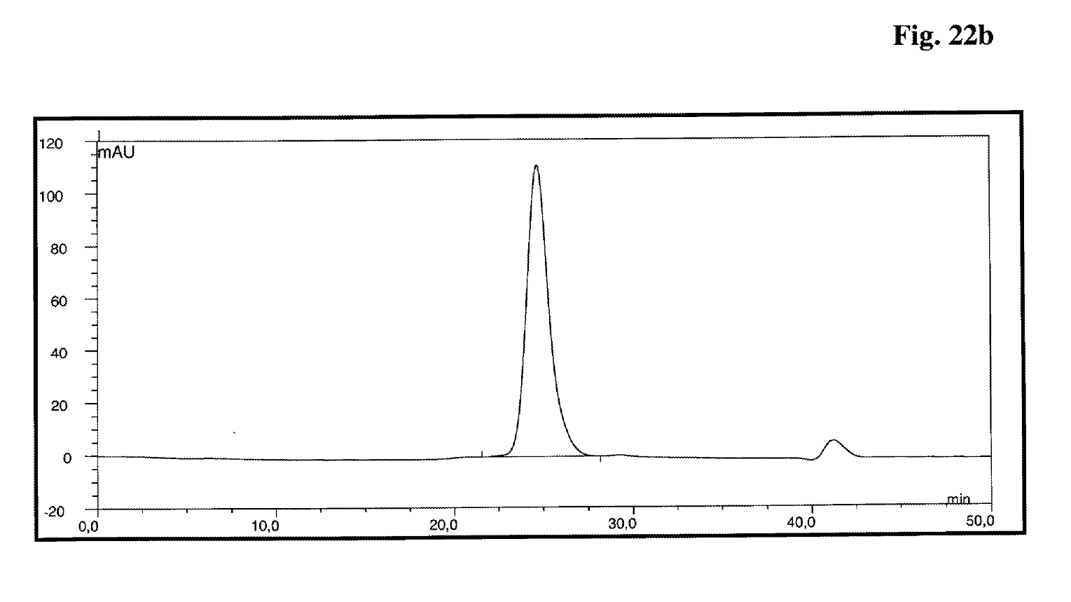

1,4-dithiotreitol) and staining with Coomassie brilliant blue (Exemplary FIG. 21 SDS-Page for bispecific Her3/c-Met antibodies Her3/MetscFvSS_KH (left side) and Her3/MetscFv_KH (right side)). The NuPAGE® Pre-Cast gel system (Invitrogen, USA) was used according to the manufacturer's instruction (4-20% Tris-Glycine gels). The aggregate content of bispecific and control antibody samples was analyzed by high-performance SEC using a Superdex 200 analytical size-exclusion column (GE Healthcare, Sweden) in 200 mM KH₂PO₄, 250 mM KCl, pH 7.0 running buffer at 25° C. 25 μg protein were injected on the column at a flow rate of 0.5 ml/min and eluted isocratic over 50 minutes. For stability analysis, concentrations of 1 mg/ml of purified proteins were incubated at 4° C. and 40° C. for 7 days and then evaluated by high-performance SEC (e.g. HP SEC Analysis (Purified Protein) of bispecific Her3/c-Met antibodies Her3/MetscFvSS_KH (FIG. 22a) and Her3/MetscFv_KH (FIG. 22b)). The integrity of the amino acid backbone of reduced bispecific antibody light and heavy chains was verified by NanoElectrospray Q-TOF mass spectrometry after removal of N-glycans by enzymatic treatment with Peptide-N-Glycosidase F (Roche Molecular Biochemicals). Yields were e.g. for the bispecific Her3/c-Met antibodies Her3/MetscFvSS_KH 28.8 mg/L (ProteinA and SEC) and Her3/MetscFv_KH 12.3 mg/L (ProteinA and SEC)).
c-Met Phosphorylation Assay

5×10e5 A549 cells were seeded per well of a 6-well plate the day prior HGF stimulation in RPMI with 0.5% FCS (fetal calf serum). The next day, growth medium was replaced for one hour with RPMI containing 0.2% BSA (bovine serum albumin). 5 μg/mL of the bispecific antibody was then added to the medium and cells were incubated for 10 minutes upon which HGF was added for further 10 minutes in a final concentration of 50 ng/mL. Cells were washed once with ice cold PBS containing 1 mM sodium vanadate upon which they were placed on ice and lysed in the cell culture plate with 100 μL lysis buffer (50 mM Tris-Cl pH7.5, 150 mM NaCl, 1% NP40, 0.5% DOC, aprotinine, 0.5 mM PMSF, 1 mM sodium-vanadate). Cell lysates were transferred to eppendorf tubes and lysis was allowed to proceed for 30 minutes on ice. Protein concentration was determined using the BCA method (Pierce). 30-50 μg of the lysate was separated on a 4-12% Bis-Tris NuPage gel (Invitrogen) and proteins on the gel were transferred to a nitrocellulose membrane. Membranes were blocked for one hour with TBS-T containing 5% BSA and developed with a phospho-specific c-Met antibody directed against Y1230, 1234, 1235 (44-888, Biosource) according to the manufacturer's instructions. Immunoblots were reprobed with an antibody binding to unphosphorylated c-Met (AF276, R&D).

### Her3 (ErbB3) Phosphorylation Assay

2×10e5 MCF7 cells were seeded per well of a 12-well plate in complete growth medium (RPMI 1640, 10% FCS). Cells were allowed to grow to 90% confluency within two days. Medium was then replaced with starvation medium containing 0.5% FCS. The next day the respective antibodies were supplemented at the indicated concentrations 1 hour prior addition of 500 ng/mL Heregulin (R&D). Upon addition of Heregulin cells were cultivated further 10 minutes before the cells were harvested and lysed. Protein concentration was determined using the BCA method (Pierce). 30-50 μg of the lysate was separated on a 4-12% Bis-Tris NuPage gel (Invitrogen) and proteins on the gel were transferred to a nitrocellulose membrane. Membranes were blocked for one hour with TBS-T containing 5% BSA and developed with a phospho-specific Her3/ErbB3 antibody specifically recognizing Tyr1289 (4791, Cell Signaling).

### Scatter Assay

A549 (4000 cells per well) or A431 (8000 cells per well) were seeded the day prior compound treatment in a total volume of 200 μL in 96-well E-Plates (Roche, 05232368001) in RPMI with 0.5% FCS. Adhesion and cell growth was monitored over night with the Real Time Cell Analyzer machine with sweeps every 15 min monitoring the impedance. The next day, cells were pre-incubated with 5 μL of the respective antibody dilutions in PBS with sweeps every five minutes. After 30 minutes 2.5 μL of a HGF solution yielding a final concentration of 20 ng/mL were added and the experiment was allowed to proceed for further 72 hours. Immediate changes were monitored with sweeps every minute for 180 minutes followed by sweeps every 15 minutes for the remainder of the time.

### Migration Assay

Migration assays were performed based on the Real Time Cell Analyzer Technology (Roche). For this purpose, the lower chamber of CIM devices with 8 μm pores were filled with 160 μL of HGF-conditioned media (50 ng/mL). The device was assembled and 100000 A431 cells in a total volume of 150 μL were seeded in the upper chamber. To this, bispecific antibodies or control antibodies were added. Migration was allowed to proceed for 24 h with regular sweeps every 15 min in between. Data was exported and is presented as an endpoint readout after 24 h.

### Flow Cytometry Assay (FACS)

### a) Relative Quantitation of Cell Surface Receptor Status

Cells were maintained in the logarithmic growth phase. Subconfluent cells were detached with accutase (Sigma), spun down (1500 rpm, 4° C., 5 min) and subsequently washed once with PBS containing 2% FCS. To determine the relative receptor status in comparison to other cell lines, 1×10e5 cells were either incubated with 5 μg/mL of Her3 or c-Met specific primary antibody for 30 min on ice. As specificity control an unspecific IgG (isotype control) was used. After the indicated time, cells were washed once with PBS containing 2% FCS followed by an incubation with a fluorophor coupled secondary antibody for 30 min on ice. Cells were washed as described and resuspended in an appropriate volume of BD CellFix solution (BD Biosciences) containing 7-AAD (BD Biosciences) to discriminate living and dead cells. Mean fluorescence intensity (mfi) of the cells was determined by flow cytometry (FACS Canto, BD). Mfi was determined at least in duplicates of two independent stainings. Flow cytometry spectra were further processed using the FlowJo software (TreeStar).

### a) Binding Assay

A431 were detached and counted. 1.5×10e5 cells were seeded per well of a conical 96-well plate. Cells were spun down (1500 rpm, 4° C., 5 min) and incubated for 30 min on ice in 50 μL of a dilution series of the respective bispecific antibody in PBS with 2% FCS (fetal calf serum). Cells were again spun down and washed once with 200 μL PBS containing 2% FCS followed by a second incubation of 30 min with a phycoerythrin-coupled antibody directed against human Fc which was diluted in PBS containing 2% FCS (Jackson Immunoresearch, 109116098). Cells were spun down washed twice with 200 μL PBS containing 2% FCS, resuspended in BD CellFix solution (BD Biosciences) and incubated for at least 10 min on ice. Mean fluorescence intensity (mfi) of the cells was determined by flow cytometry (FACS Canto, BD). Mfi was determined at least in duplicates of two independent stainings Flow cytometry spectra were further processed using the FlowJo software (TreeStar). Half-maximal binding was determined using XLFit 4.0 (IDBS) and the dose response one site model 205.

### b) Internalization Assay

Cells were detached and counted. 5×10e5 cells were placed in 50 μL complete medium in an eppendorf tube and incubated with 5 μg/mL of the respective bispecific antibody at 37° C. After the indicated time points cells were stored on ice until the time course was completed. Afterwards, cells were transferred to FACS tubes, spun down (1500 rpm, 4° C., 5 min), washed with PBS+2% FCS and incubated for 30 minutes in 50 μL phycoerythrin-coupled secondary antibody directed against human Fc which was diluted in PBS containing 2% FCS (Jackson Immunoresearch, 109116098). Cells were again spun down, washed with PBS+2% FCS and fluorescence intensity was determined by flow cytometry (FACS Canto, BD).

### c) Crosslinking Experiment

HT29 cells were detached counted and split in two populations which were individually stained with PKH26 and PKH67 (Sigma) according to the manufacturer's instructions. Of each of the stained populations 5×10e5 cells were taken, combined and incubated for 30 and 60 minutes with 10 μg/mL of the respective bispecific antibody in complete medium. After the indicated time points cells were stored on ice until the time course was completed. Cells were spun down (1500 rpm, 4° C., 5 min), washed with PBS+2% FCS and fluorescence intensity was determined by flow cytometry (FACS Canto, BD).

### Cell Titer Glow Assay

Cell viability and proliferation was quantified using the cell titer glow assay (Promega). The assay was performed according to the manufacturer's instructions. Briefly, cells were cultured in 96-well plates in a total volume of 100 μL for the desired period of time. For the proliferation assay, cells were removed from the incubator and placed at room temperature for 30 min. 100 μL of cell titer glow reagent were added and multi-well plates were placed on an orbital shaker for 2 min. Luminescence was quantified after 15 min on a microplate reader (Tecan).

### Wst-1 Assay

A Wst-1 viability and cell proliferation assay was performed as endpoint analysis, detecting the number of metabolic active cells. Briefly, 20 μL of Wst-1 reagent (Roche, 11644807001) were added to 200 μL of culture medium. 96-well plates were further incubated for 30 min to 1 h until robust development of the dye. Staining intensity was quantified on a microplate reader (Tecan) at a wavelength of 450 nm.

### Surface Plasmon Resonance

The binding affinity is determined with a standard binding assay at 25° C., such as surface plasmon resonance technique (BIAcore®, GE-Healthcare Uppsala, Sweden). For affinity measurements, 30 μg/ml of anti Fcγ antibodies (from goat, Jackson Immuno Research) were coupled to the surface of a CM-5 sensor chip by standard amine-coupling and blocking chemistry on a SPR instrument (Biacore T100). After conjugation, mono- or bispecific Her3/c-Met antibodies were injected at 25° C. at a flow rate of 5 μL/min, followed by a dilution series (0 nM to 1000 nM) of human HER3 or c-Met ECD at 30 μL/min. As running buffer for the binding experiment PBS/0.1% BSA was used. The chip was then regenerated with a 60 s pulse of 10 mM glycine-HCl, pH 2.0 solution.

Design of Expressed and Purified Bispecific <ErbB3-c-Met> Antibodies

All of the following expressed and purified bispecific <ErbB3-c-Met> antibodies comprise a constant region or at least the Fc part of IgG1 subclass (human constant IgG1 region of SEQ ID NO: 11) which is eventually modified as indicated below.

In Table 1: Trivalent, bispecific <ErbB3-c-Met> antibodies based on a full length ErbB-3 antibody (HER3 clone29) obtained via immunization (NMRI mice immunized with human HER3-ECD) and one single chain Fv fragment (for a basic structure scheme see FIG. 5,—eventually not all features mentioned in the Table are include in the figure) from a c-met antibody (c-Met 5D5) with the respective features shown in Table 1 one were expressed and purified according to the general methods described above. The corresponding VH and VL of HER3 clone29 and c-Met 5D5 are given in the sequence listing.

TABLE 1

| | Molecule Name scFv-Ab-nomenclature for bispecific antibodies | | | | |
|---|---|---|---|---|---|
| Features: | Her3/Met_scFvSSKHSS | Her3/Me_scFvSSKH | Her3/Met_scFvKH | Her3/Me_scFvKHSB | Her3/Met_scFvKHSBSS |
| Knobs-in-hole | S354C:T366W/ | T366W/ | T366W/ | T366W:K370E: | S354C:T366W:K370E: |
| mutations | Y349′C:T366′S: | T366′S:L368′A:Y407′V | T366′S:L368′A:Y407′V | K409D/ | K409D/ |
| | L368′A:Y407′V | | | E357′K:T366′S: | Y349′C:E357′K:T366′S: |
| | | | | L368′A:D399′K: | L368′A:D399′K: |
| | | | | Y407′V | Y407′V |
| Full length | Her3 | Her3 | Her3 | Her3 | Her3 |
| antibody | clone 29 | clone 29 | clone 29 | clone 29 | clone 29 |
| backbone derived | (chimeric) | (chimeric) | (chimeric) | (chimeric) | (chimeric) |
| from | | | | | |
| Single chain Fv | c-Met 5D5 | c-Met 5D5 | c-Met 5D5 | c-Met 5D5 | c-Met 5D5 |
| fragment derived | (humanized) | (humanized) | (humanized) | (humanized) | (humanized) |
| from | | | | | |
| Position of scFv | C-terminus | C-terminus knob | C-terminus | C-terminus | C-terminus knob |
| attached to | knob heavy | heavy chain | knob heavy | knob heavy | heavy chain |
| antibody | chain | | chain | chain | |
| single-chain-Fv- | (G₄S)₃ | (G₄S)₃ | (G₄S)₃ | (G₄S)₃ | (G₄S)₃ |
| linker | | | | | |
| Peptide connector | (G₄S)₂ | (G₄S)₂ | (G₄S)₂ | (G₄S)₂ | (G₄S)₂ |
| ScFv disulfide | + | + | − | + | + |
| VH44/VL100 | | | | | |
| stabilized(Yes/no = | | | | | |
| +/−) | | | | | |

In Table 2: Trivalent, bispecific <ErbB3-c-Met> antibodies based on a full length ErbB-3 antibody (Mab 205, obtained via immunization (NMRI mice immunized with human HER3-ECD)) and one single chain Fv or scFab fragment (for a basic structure scheme see FIG. 5 b and a—for detailed structure see Table) from a c-met antibody (c-Met 5D5) with the respective features shown in Table 2 were expressed and purified according to the general methods described above. The corresponding VH and VL of Mab205 and c-Met 5D5 are given in the sequence listing.

TABLE 2

| | Molecule Name scFv-Ab-nomenclature for bispecific antibodies | | | | | |
|---|---|---|---|---|---|---|
| Features: | MH_TvAB18 | MH_TvAB21 | MH_TvAB22 | MH_TvAB23 | MH_TvAB24 | MH_TvAB25 |
| Knobs-in-hole | S354C:T366W/ | S354C:T366W/ | S354C:T366W/ | S354C:T366W/ | S354C:T366W/ | S354C:T366W/ |
| mutations | Y349′C:T366′S: | Y349′C:T366′S: | Y349′C:T366′S: | Y349′C:T366′S: | Y349′C:T366′S: | Y349′C:T366′S: |
| | L368′A:Y407′V | L368′A:Y407′V | L368′A:Y407′V | L368′A:Y407′V | L368′A:Y407′V | L368′A:Y407′V |
| Full length | Mab | Mab 205 | Mab 205 | Mab 205 | Mab | Mab |
| antibody | 205 | (chimeric) | (chimeric) | (chimeric) | (chimeric) | (chimeric) |
| backbone derived | (chimeric) | | | | | |
| from | | | | | | |
| Single chain Fv | c-Met 5D5 | — | — | c-Met 5D5 | c-Met 5D5 | c-Met 5D5 |
| fragment derived | (humanized) | | | (humanized) | (humanized) | (humanized) |
| from | | | | | | |
| Single chain | — | c-Met 5D5 | c-Met 5D5 | — | — | — |
| scFab fragment | | (humanized) | (humanized) | | | |
| derived from | | | | | | |
| Position of scFv | C-terminus | C-terminus | N-terminus | N-terminus | N-terminus | N-terminus |
| or scFab | knob heavy | knob heavy | knob heavy | knob heavy | knob heavy | knob heavy |
| attached to | chain | chain | chain | chain | chain | chain |
| antibody | | | | | | |
| single-chain-Fv- | (G₃S)₄ | — | — | (G₃S)₄ | (G₃S)₇ | (G₃S)₇ |
| linker | | | | | | |
| linker (scFab) | — | (G₄S)₅ GG | (G₄S)₅ GG | — | — | — |
| Peptide | (G₄S)₂ | (G₄S)₂ | (G₄S)₂ | (G₄S)₂ | (G₄S)₂ | (G₄S)₂ |
| connector | | | | | | |
| ScFv or ScFab | + | − | − | + | − | − |
| disulfide VH44/ | | | | | | |
| VL100 stabilized | | | | | | |
| (Yes/no = +/−) | | | | | | |

In Table 3: Trivalent, bispecific <ErbB3-c-Met> antibodies based on a full length ErbB-3 antibody (Mab 205.10.2, obtained via immunization (NMRI mice immunized with human HER3-ECD) and subsequent humanization) and one scFab fragment (for a basic structure scheme see FIG. 5a) from a c-met antibody (c-Met 5D5) with the respective features shown in Table 3 were expressed and purified according to the general methods described above. The corresponding VH and VL of Mab 205.10.2 and c-Met 5D5 are given in the sequence listing.

TABLE 3

Molecule Name scFv-Ab-nomenclature
for bispecific antibodies

| Features: | MH_TvAB29 | MH_TvAB30 |
|---|---|---|
| Knobs-in-hole | S354C:T366W/ | S354C:T366W/ |
| mutations | Y349′C:T366′S: | Y349′C:T366′S: |
| | L368′A:Y407′V | L368′A:Y407′V |
| Full length | Mab | Mab |
| antibody | 205.10.2 | 205.10.2 |
| backbone derived | (humanized) | (humanized) |
| from | | |
| scFab fragment | c-Met 5D5 | c-Met 5D5 |
| derived from | (humanized) | (humanized) |
| Position of scFv | C-terminus | C-terminus knob |
| attached to | knob heavy | heavy chain |
| antibody | chain | |
| linker (scFab) | ((G₄S)₅ GG | (G₄S)₅ GG |
| Peptide | (G₄S)₂ | (G₄S)₂ |
| connector | | |
| ScFv or ScFab | + | − |
| disulfide VH44/ | | |
| VL100 stabilized | | |
| (Yes/no = +/−) | | |

In Table 4: Trivalent, bispecific <ErbB3-c-Met> antibodies based on a full length ErbB-3 antibody (HER3 clone29) and the VH and VL domain (for a basic structure scheme see FIGS. 3a, 3c, and 3 d—eventually not all features mentioned in the Table are include in the figures) from a c-Met antibody (c-Met 5D5) with the respective features shown in Table 4 were expressed and purified according to the general methods described above. The corresponding VH and VL of HER3 clone29 and c-Met 5D5 are given in the sequence listing.

TABLE 4
Trivalent, bispecific antibody with the VHVL-Ab-nomenclature in Table 2 were
expressed and purified (see also in the Examples below and FIG. 3c)

| | Molecule Name VHVL-Ab-nomenclature for bispecific antibodies | | | | |
|---|---|---|---|---|---|
| Features: | Her3/Met_KHSS | Her3/Met_SSKH | Her3/Met_SSKHSS | Her3/Met_1C | Her3/Met_6C |
| Knobs-in-hole | S354C:T366W/ | T366W/ | S354C:T366W/ | S354C:T366W/ | S354C:T366W/ |
| mutations | Y349′C:T366′S: | T366′S:L368′A:Y407′V | Y349′C:T366′S: | Y349′C:T366′S: | Y349′C:T366′S: |
| | L368′A:Y407′V | | L368′A:Y407′V | L368′A:Y407′V | L368′A:Y407′V |
| Full length | Her3 | Her3 | Her3 | Her3 | Her3 |
| antibody | clone 29 | clone 29 | clone 29 | clone 29 | clone 29 |
| backbone derived | (chimeric) | (chimeric) | (chimeric) | (chimeric) | (chimeric) |
| from | | | | | |
| VHVL fragment | c-Met 5D5 | c-Met 5D5 | c-Met 5D5 | c-Met 5D5 | c-Met 5D5 |
| derived from | (humanized) | (humanized) | (humanized) | (humanized) | (humanized) |
| Position of VH | C-terminus | C-terminus knob | C-terminus knob | C-terminus | C-terminus |
| attached to | knob heavy | heavy chain | heavy chain | knob heavy | knob heavy |
| antibody | chain | | | chain | chain |
| Position of VL | C-terminus | C-terminus hole | C-terminus hole | C-terminus | C-terminus |
| attached to | hole heavy | heavy chain | heavy chain | hole heavy | hole heavy |
| antibody | chain | | | chain | chain |
| Peptide | (G₄S)₃ | (G₄S)₃ | (G₄S)₃ | (G₄S)₁ | (G₄S)₆ |
| connector | | | | | |
| VHVL disulfide | − | + | + | − | − |
| VH44/VL100 | | | | | |
| stabilized (Yes/no = | | | | | |
| +/−) | | | | | |

In Table 5: Bivalent, bispecific <ErbB3-c-Met> antibodies wherein one binding arm is based on a full length ErbB-3 antibody (HER3Mab 205 or humanized versions Mab 205.10.1, Mab 205.10.2 or Mab 205.10.2) and the other binding arm is based on a scFab fragment from a c-met antibody (c-Met 5D5) with the respective features shown in Table 5 (for a basic structure scheme see FIG. 7) were expressed and purified according to the general methods described above. The corresponding VH and VL of HER3Mab 205 and c-Met 5D5 are given in the sequence listing.

TABLE 5

Molecule Name scFv-Ab-nomenclature
for bispecific antibodies

| Features: | MH_BvAB21 | MH_BvAB28 |
|---|---|---|
| Knobs-in-hole | S354C:T366W/ | S354C:T366W/ |
| mutations | Y349′C:T366′S: | Y349′C:T366′S: |
| | L368′A:Y407′V | L368′A:Y407′V |
| Full length | Mab205 | Mab205.10.2 |
| antibody | (chimeric) | (humanized) |
| backbone derived | | |
| from | | |
| Single chain Fab | c-Met 5D5 | c-Met 5D5 |
| fragment derived | (humanized) | (humanized) |
| from | | |
| Position of scFab | N-terminus of | N-terminus of |
| attached to | knob-CH2—CH3 | knob-CH2—CH3 |
| | fragment | fragment |
| single-chain- | (G₄S)₅ GG | (G₄S)₅ GG |
| Fab-linker | | |
| ScFab disulfide | − | + |
| VH44/VL100 | | |
| stabilized (Yes/no = | | |
| +/−) | | |

Example 1

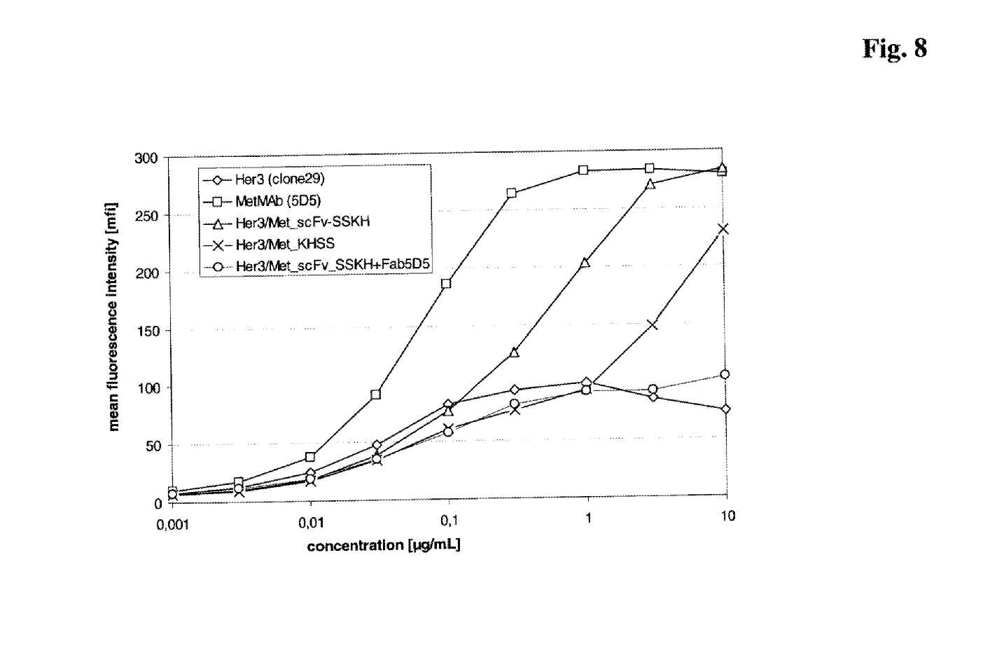

### FIG. 8

### Binding of Bispecific Antibodies to the Cell Surface of Cancer Cells

The binding properties of the bispecific antibodies to their respective receptor on the cell surface was analyzed on A431 cancer cells in a flow cytometry based assay. Cells were incubated with the mono- or bispecific primary antibodies and binding of these antibodies to their cognate receptors was detected with a secondary antibody coupled to a fluorophor binding specifically to the Fc of the primary antibody. The mean fluorescence intensity of a dilution series of the primary antibodies was plotted against the concentration of the antibody to obtain a sigmoidal binding curve. Cell surface expression of c-Met and Her3 was validated by incubation with the bivalent 5D5 and Her3 clone 29 antibody only. The Her3/c-Met KHSS antibody readily bind to the cell surface of A431. Under these experimental settings, the antibody can only bind via its Her3 part and consequently the mean fluorescence intensity does not exceed the staining for Her3 clone 29 alone.

### Example 2

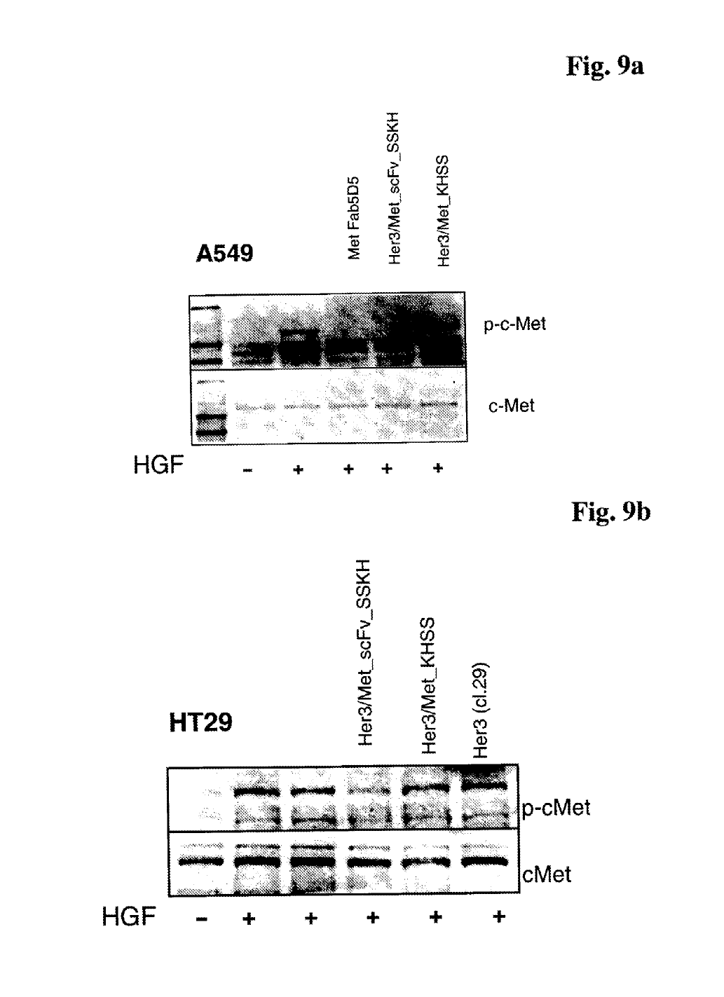

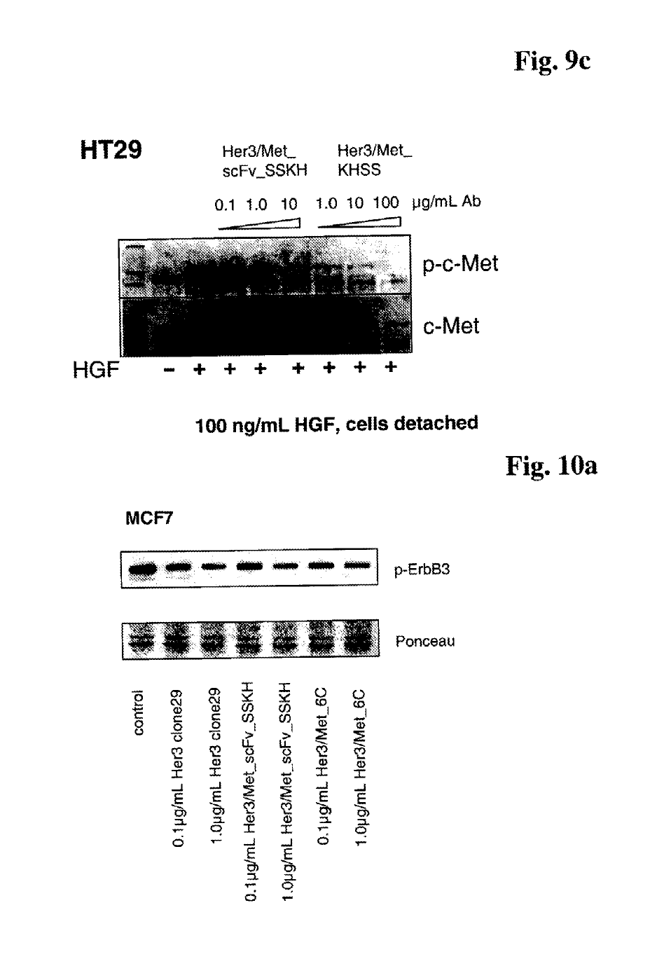

### FIG. 9

### Inhibition of HGF-Induced c-Met Receptor Phosphorylation by Bispecific her3/c-Met Antibody Formats

To confirm functionality of the c-Met part in the bispecific antibodies a c-Met phosphorylation assay was performed. In this experiment A549 lung cancer cells or HT29 colorectal cancer cells were treated with the bispecific antibodies or control antibodies prior exposure to HGF. Cells were then lysed and phosphorylation of the c-Met receptor was examined. Both cell lines can be stimulated with HGF as can be observed by the occurrence of a phospho-c-Met specific band in the immunoblot. Addition of the scFv antibody or the 5D5 Fab fragment inhibits receptor phosphorylation demonstrating functionality of the c-Met scFv component.

### Example 3

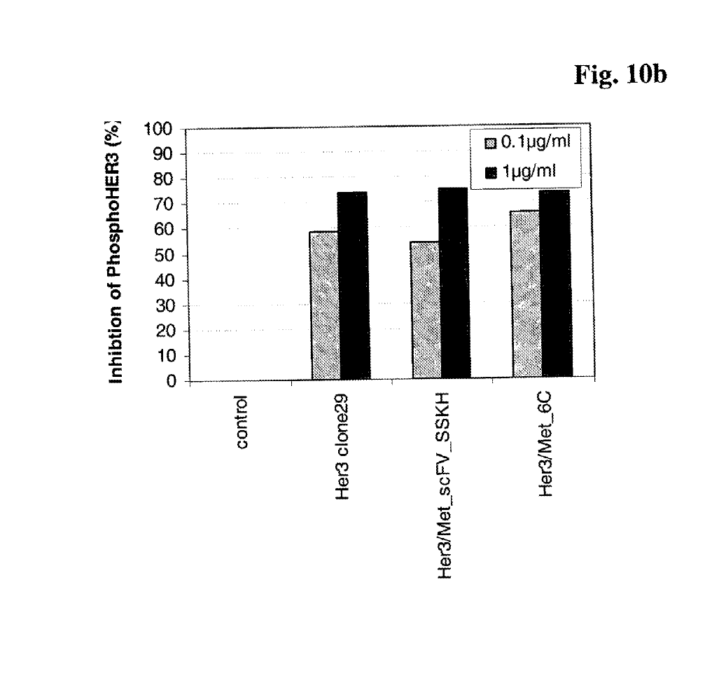

### FIG. 10

### Inhibition of HRG-Induced Her3 Receptor Phosphorylation by Bispecific Her3/c-Met Antibody Formats

To confirm functionality of the Her3 part in the bispecific antibodies a Her3 phosphorylation assay was performed. In this experiment MCF7 cells were treated with the bispecific antibodies or control antibodies prior exposure to HRG (Heregulin). Cells were then lysed and phosphorylation of the Her3 receptor was examined. Her3/c-Met_scFV_SSKH and Her3/c-Met_KHSS inhibit Her3 receptor phosphorylation to the same extent as the parental Her3 clone29 indicating that Her3 binding and functionality of the antibody are not compromised by the trivalent antibody format.

### Example 4

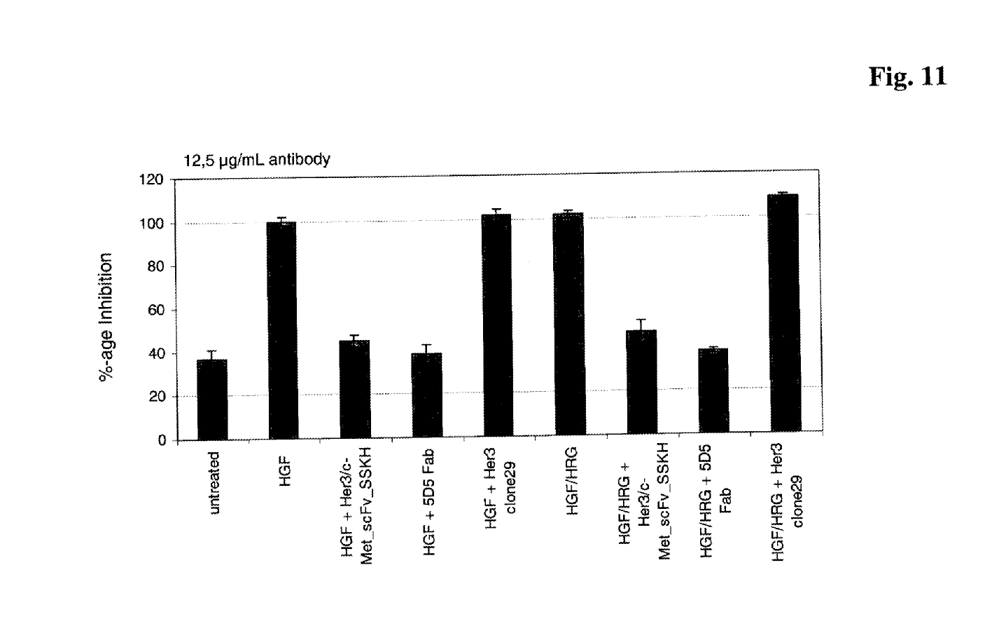

### FIGS. 11,12,13

### Inhibition of HGF-Induced HUVEC Proliferation by Bispecific Her3/c-Met Antibody Formats

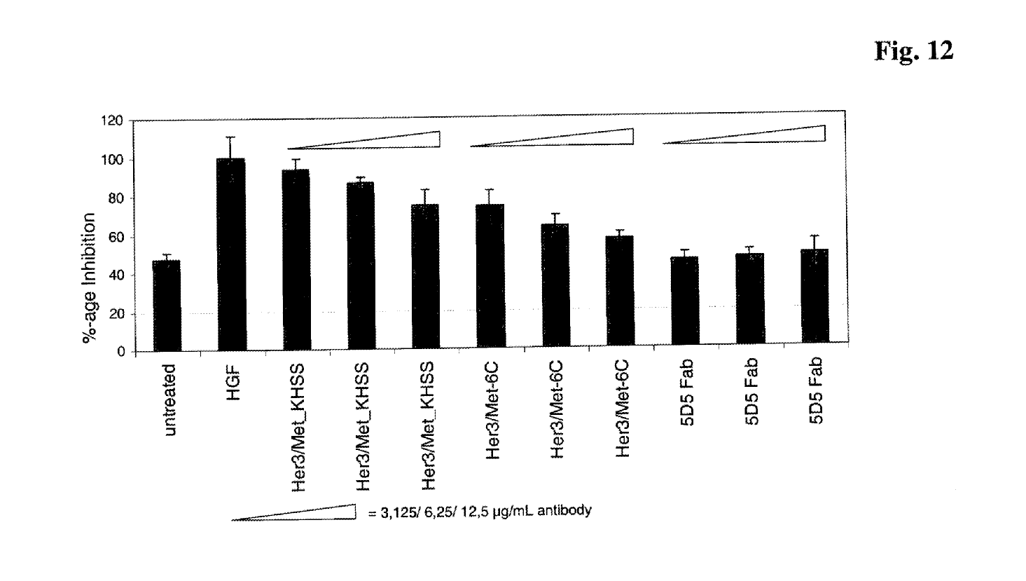

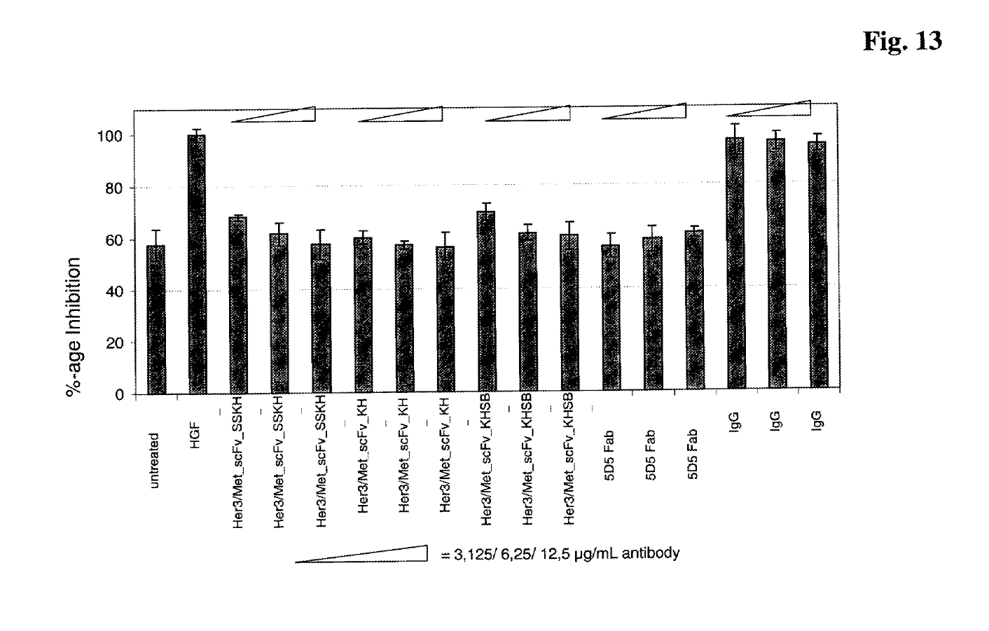

HUVEC proliferation assays can be performed to demonstrate the mitogenic effect of HGF. Addition of HGF to HUVEC leads to a twofold increase in proliferation. Addition of human IgG control antibody in the same concentration range as the bispecific antibodies has no impact on cellular proliferation while the 5D5 Fab fragment inhibits HGF-induced proliferation. If used at the same concentration, the Her3/c-Met_scFv_SSKH antibody inhibits proliferation as good as the Fab fragment (FIG. 11). Heregulin (HRG) addition alone (data not shown) or in combination with HGF results in no further increase of proliferation (FIG. 12). This confirms that this readout allows the functional analysis of the c-Met component in the bispecific antibody format without interference of the Her3 component. Titration of Her3/c-Met KHSS demonstrate a weak inhibitory effect of the antibody (FIG. 13). The effect is more pronounced for the Her3/Met-6C antibody indicating that a longer connector improves efficacy of the antibody. Three different scFv antibodies (Her3/c-Met_scFv_SSKH, Her3/c-Met_scFv_KH, Her3/c-Met_scFv_KHSB) exhibit the same degree of proliferation inhibition. This demonstrates the functionality of the c-Met component in the trivalent antibody format.

### Example 5

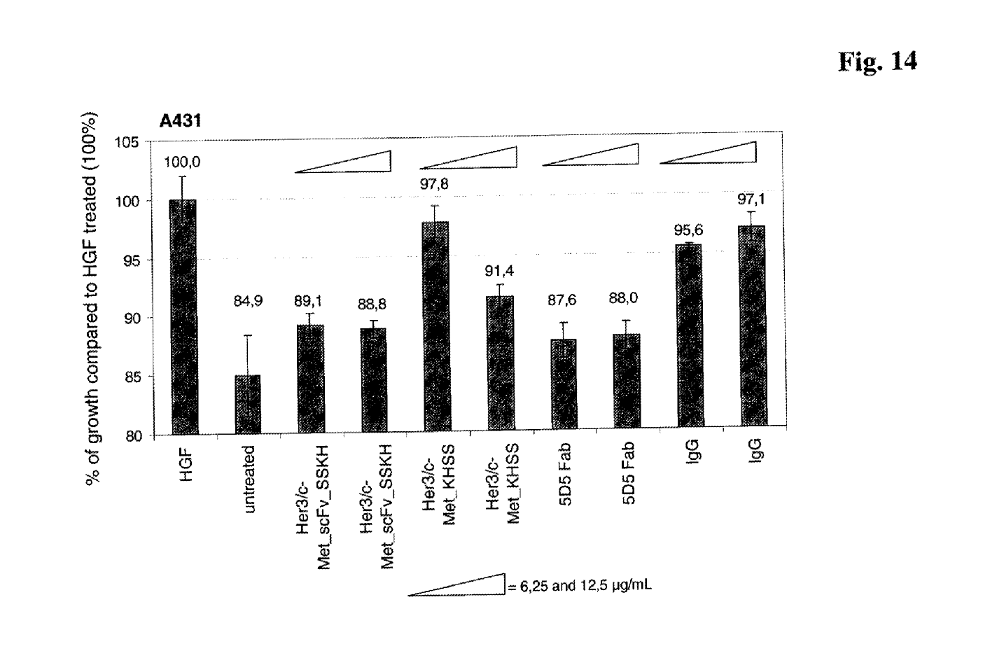

### FIG. 14

### Inhibition of Proliferation in the Cancer Cell Line A431 by Bispecific Her3/c-Met Antibody Formats

If A431 are seeded in serum reduced medium, addition of HGF induces apart from scattering a weak mitogenic effect. This was exploited to analyze the impact of Her3/c-Met_scFv_SSKH and Her3/c-Met_KHSS on HGF treated A431 proliferation. Indeed, the bispecific antibodies can largely inhibit the HGF-induced increase of proliferation (15%). Her3/c-Met_scFv_SSKH is as good as the 5D5 Fab fragment while Her3/c-Met_KHSS has to be dosed higher (12.5 μg/mL in contrast to 6.25 μg/mL) to obtain similar effects. A control human IgG1 antibody has no influence on HGF promoted A431 cell growth.

### Example 6

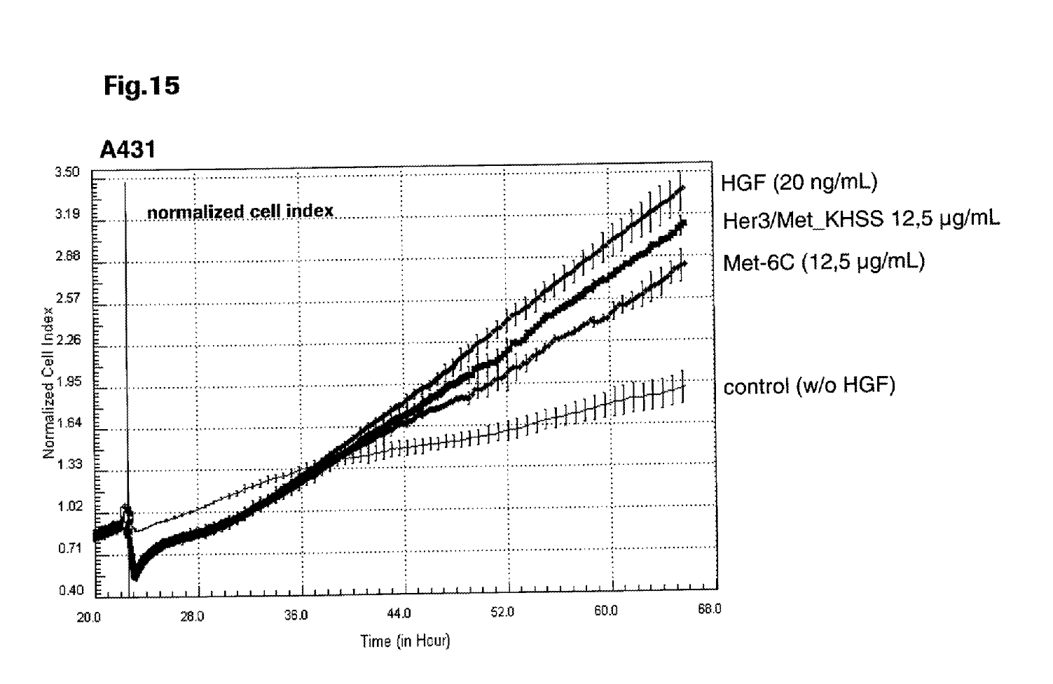

### FIGS. 15,16

### Analysis of Inhibition of HGF-Induced Cell-Cell Dissemination (Scattering) in the Cancer Cell Line A431 by Bispecific Her3/c-Met Antibody Formats

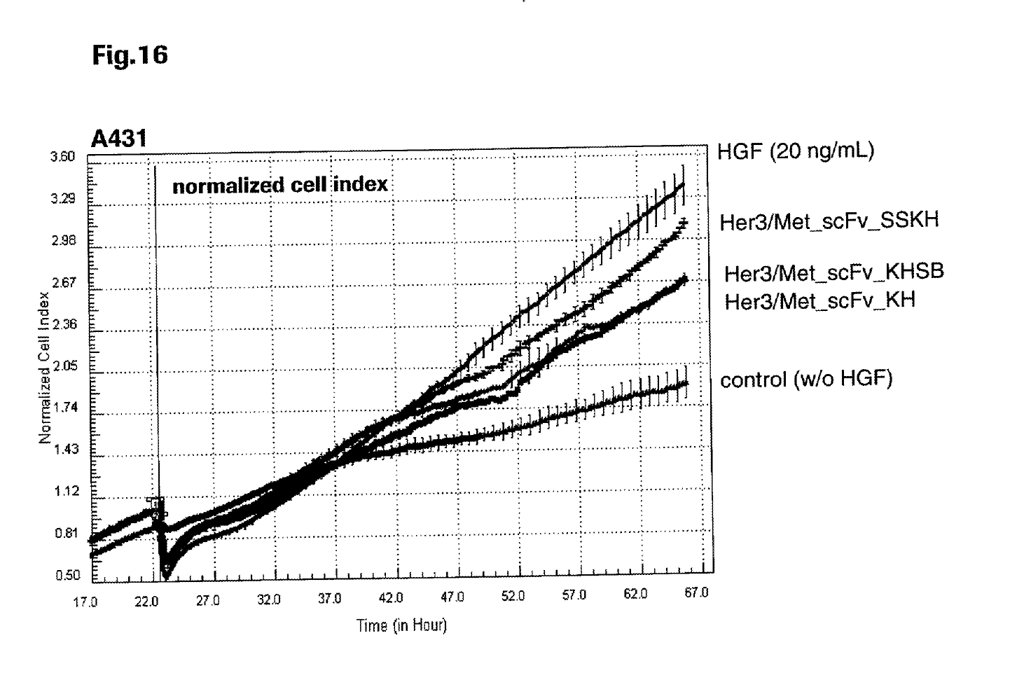

HGF-induced scattering includes morphological changes of the cell, resulting in rounding of the cells, filopodia-like protrusions, spindle-like structures and a certain motility of the cells. The Real Time Cell Analyzer (Roche) measures the impedance of a given cell culture well and can therefore indirectly monitor changes in cellular morphology and proliferation. Addition of HGF to A431 and A549 cells results in changes of the impedance which can be monitored as function of time. Her3/c-Met_KHSS and Her3/Met-6C inhibit HGF-induced scattering with Her3/Met-6C being more efficacious (20.7% and 43.7% scatter inhibition) (FIG. 15). Three different scFv antibodies (Her3/c-Met_scFv_SSKH, Her3/c-Met_scFv_KH, Her3/c-Met_scFv_KHSB) display medium efficacy in suppressing HGF-induced scattering as can be observed by the reduced slope of the curve drawing near the untreated control curve (29%, 51.9% and 49.7% scatter inhibition) (FIG. 16). If used at the same concentration of 12.5 μg/mL the Her3/c-Met_scFv_KH antibody and Her3/c-Met_scFv_KHSB perform equally well.

### Example 7

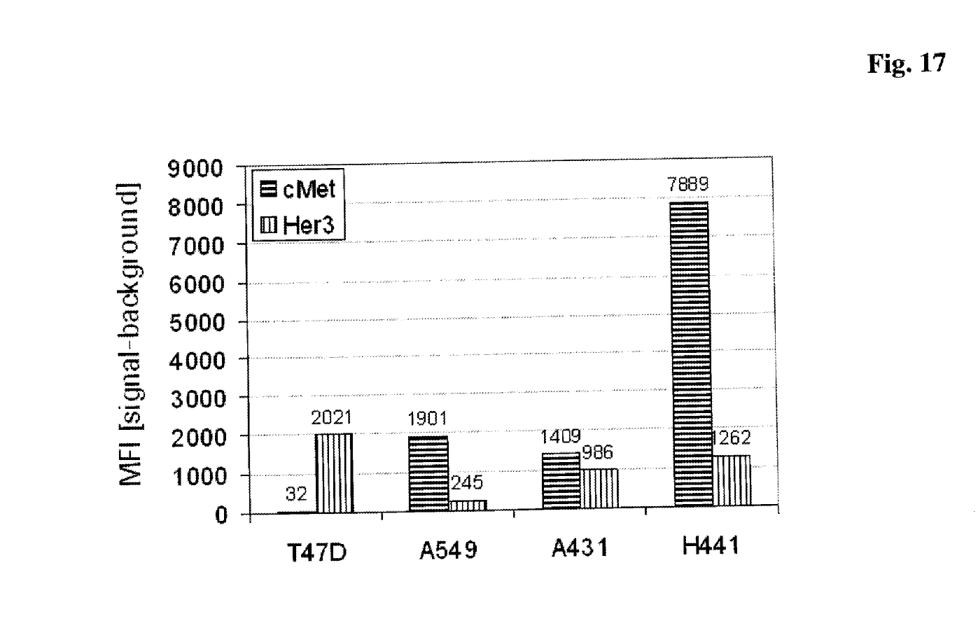

### FIG. 17

### Analysis of Cell Surface Expression of the Her3 and c-Met Receptor in the Cancer Cell Lines T47D, A549, A431, and H441

To identify cell lines with different ratios of cell surface Her3 and c-Met a FACS-based assay was performed. T47D did not show c-Met cell surface expression, which is in accordance with mRNA levels in this cell line (data not shown). A431 and A549 display similar levels of c-Met while H441, a cell line which overexpresses c-Met has very high c-Met levels. Vice versa T47D have high levels of Her3 while A549 display only low cell surface expression.

### Example 8

### FIG. 18 and Table Below

### Analysis of Antibody-Mediated Receptor Internalization in the Cancer Cell Lines A431, A549, and DU145 (Measured with Flow Cytometry Assay (FACS))

Incubation of cells with antibodies specifically binding to Her3 or c-Met has been shown to trigger internalization of the receptor. In order to assess the internalization capability of the bispecific antibodies, an experimental setup was designed to study antibody-induced receptor internalization. For this purpose, cells were incubated for different periods of time (0; 30; 60 and 120 minutes (=0 h, ½h, 1 h and 2 h) with the respective primary antibody at 37° C. Cellular processes were stopped by rapidly cooling the cells to 4° C. A secondary fluorophor-coupled antibody specifically binding to the Fc of the primary antibody was used to detect antibodies bound to the cell surface. Internalization of the antibody-receptor complex depletes the antibody-receptor complexes on the cell surface and results in decreased mean fluorescence intensity. Internalization was studied in three different cell lines (A431, A549, DU145). Incubation with Her3 clone29 demonstrates that this antibody induces receptor internalization in A431 and DU145 while the effect is less pronounced in A549 which have almost no receptor on their cell surface. Incubation with 5D5 leads to good receptor internalization in A549, DU145 and less pronounced in A431. Her3/c-Met_scFv_SSKH display almost no internalization in A549 and DU145 and only modest internalization in A431 (11% after 2 h). In summary, the scFv antibody format leads only to a very modest receptor internalization indicating that the bispecific antibody acts differently than the monospecific components which suggests a simultaneous binding of the bispecific scFv antibody to both receptors capturing them on the cell surface. Results are shown in FIG. 18 and the Table below:

TABLE 6
% Internalization of ErbB3 receptor by bispecific Her3/c-Met
antibody as compared to parent monospecific HER3 and
c-Met antibody measured with FACS assay after 2 h on
A431 cells. Measurement % of ErbB3 receptor on cell surface
measured at 0 h is set as 100% of ErbB3 receptor on cell surface.
(For the monospecific, bivalent <c-Met> parent antibody
Mab 5D5, % internalization of c-Met is calculated analogously
(see indication in brackets for B) below))

| | | % Internalization |
|---|---|---|
| | | of ErbB3 after 2 h on |
| | % ErbB3 receptor | A431 cells (ATCC |
| | on A431 | No. CRL-1555) |
| | cell surface | (=100 − % antibody |
| | measured after | on cell surface) |
| | 2 h (% c-Met | (% internalization |
| | for <c-Met> | of c-Met for |
| Antibody | Mab5D5) | <c-Met> Mab5D5) |
| A) Monospecific <ErbB3 > | | |
| parent antibodies | | |
| <ErbB3> Mab 205 (chimeric) | 60 | 40 |
| <ErbB3> HER3 clone 29 | 44 | 54 |
| B) Monospecific <c-Met> | | |
| parent antibody | | |
| Mab 5D5 | (61 (% c-Met | (39 (% c-Met |
| | receptor)) | internalization) |
| C) Bispecific <ErbB3-c-Met> | | |
| antibodies | | |
| MH_TvAb_18 | 101 | −1 |
| MH_BvAb_20 | 103 | −3 |
| MH_TvAb_21 | 99 | 1 |
| MH_TvAb22 | 99 | 1 |
| MH_TvAb23 | 89 | 11 |
| MH_TvAb24 | 90 | 10 |
| MH_TvAb25 | 89 | 11 |
| MH_BvAb28 | 102 | −2 |
| MH_TvAb29 | 95 | 5 |
| MH_TvAb30 | 95 | 5 |
| Her3/Met_6C | 94 | 6 |
| Her3/Met_SSKH | 89 | 11 |

### Example 10

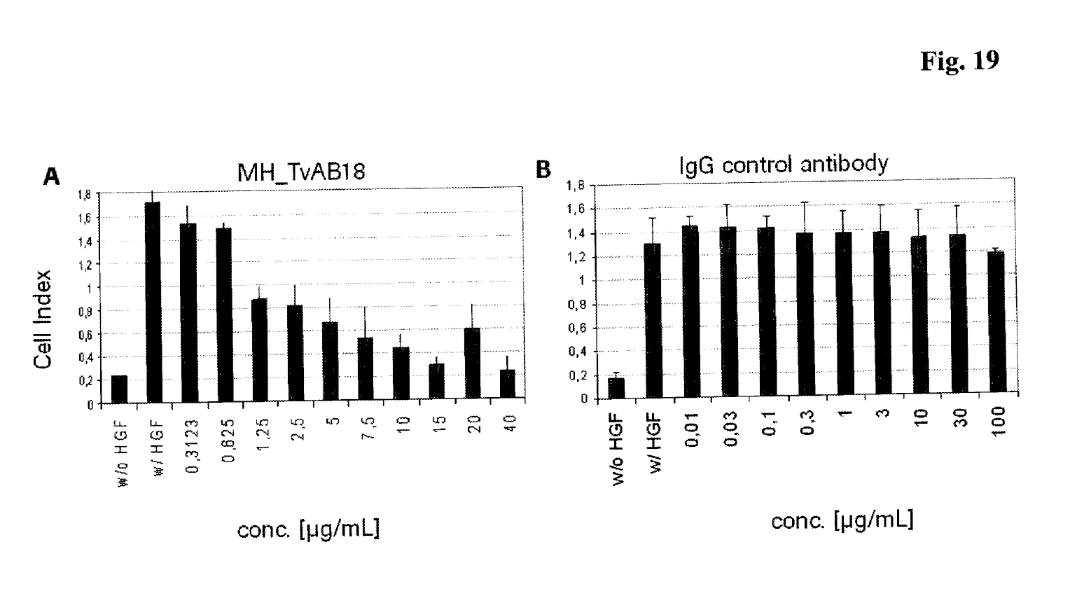

### FIG. 19

### Analysis of Antibody-Dependent Inhibition of HGF-Mediated Migration in the Cancer Cell Line A431

One important aspect of active c-Met signaling is induction of a migratory and invasive programme. Efficacy of a c-Met inhibitory antibody can be determined by measuring the inhibition of HGF-induced cellular migration. For this purpose, the HGF-inducible cancer cell line A431 was treated with HGF in the absence or presence of bispecific antibody or an IgG control antibody and the number of cells migrating through an 8 μm pore was measured in a time-dependent manner on an Acea Real Time cell analyzer using CIM-plates with an impedance readout. Independently, migration of cells was qualitatively visualized by staining the migrated cells (data not shown). The example demonstrates dose-dependent inhibition of HGF-induced cellular migration.

### Example 11

### Table Below

### Analysis of Sequential and Simultaneous Binding of Recombinant Her3, c-Met and FcgammaIII Receptor to Bispecific Antibodies

To better understand the mode of action of bispecific antibodies binding to Her3 and c-Met the receptor binding state was determined with the help of surface plasmon resonance measurements (Biacore). Different experimental setups were employed to assess binding of the bispecific antibodies to either recombinant Her3 or recombinant c-Met ectodomain (ECD) or both simultaneously. All of the tested bispecific antibodies were able to bind to Her3 and c-Met ECD simultaneously. Furthermore, binding of recombinant FcgammaIII protein to the complex of antibody:Her3:c-Met-ECD was determined. All of the antibodies could bind to the FcgammaIII receptor even in the presence of both ectodomains which provides a strong rationale for glycoengineering of the bispecific antibodies to enhance NK-dependent effector functions.

TABLE 7

| | Simul- | | | | |
|---|---|---|---|---|---|
| | taneous | | | | Simul- |
| | Binding | Affinity | Affinity | | taneous |
| | to both | c-Met | HER3 | FcgRIIIa | FcgRIIIa |
| Antibody | receptors | [nM] | [nM] | Binding | Binding |
| MH_BvAb_20 | yes | 1.2 | 0.9 | + | yes |
| MH_TvAb_21 | yes | 0.8 | 1.8 | ++ | yes |
| MH_TvAb22 | yes | 0.9 | 2.1 | + | yes |
| MH_TvAb23 | yes | 1.8 | 1.1 | + | yes |
| MH_TvAb24 | yes | 1.3 | 1.6 | + | yes |
| MH_TvAb25 | yes | 1.3 | 1.2 | + | yes |
| MH_TvAb30 | yes | 1.4 | 1.3 | +++ | yes |

### Example 12

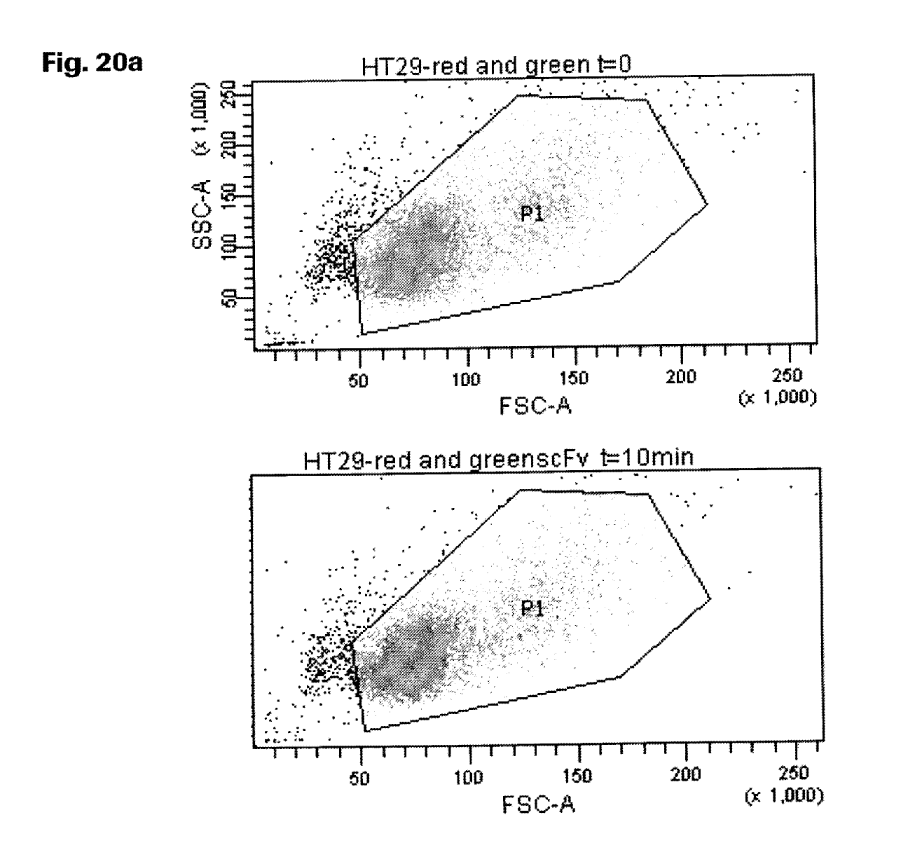

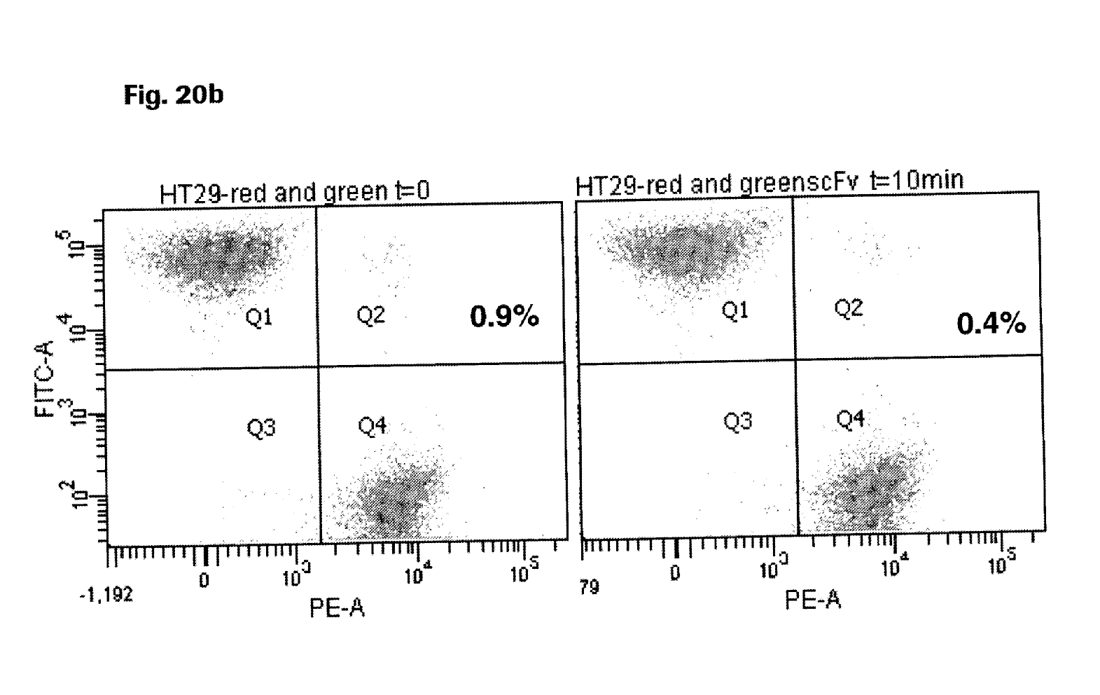

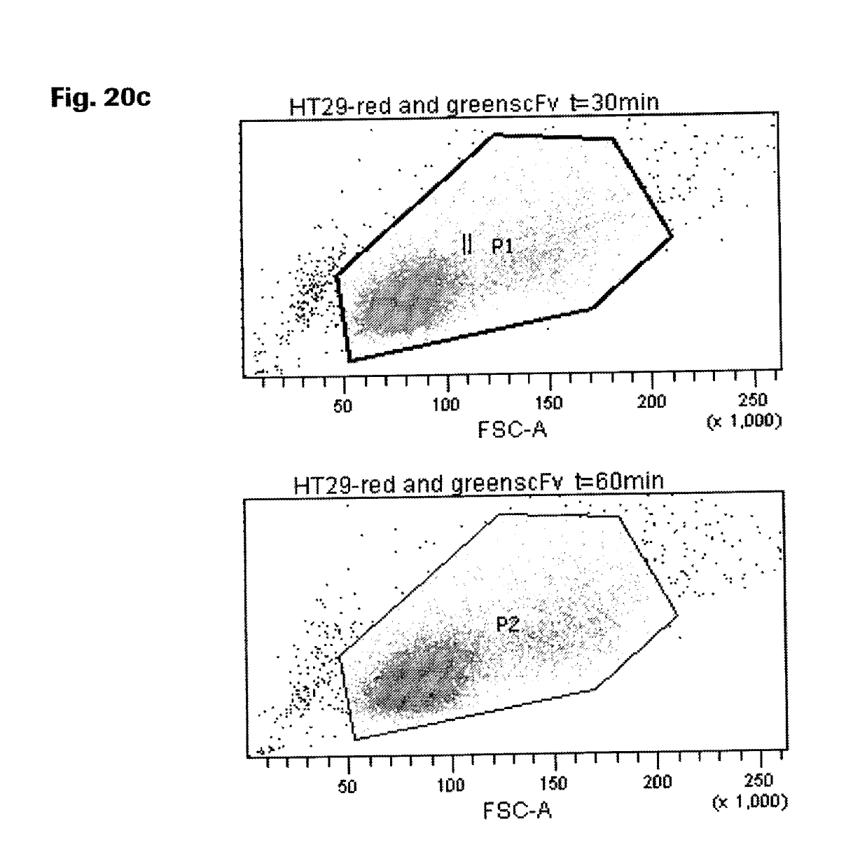

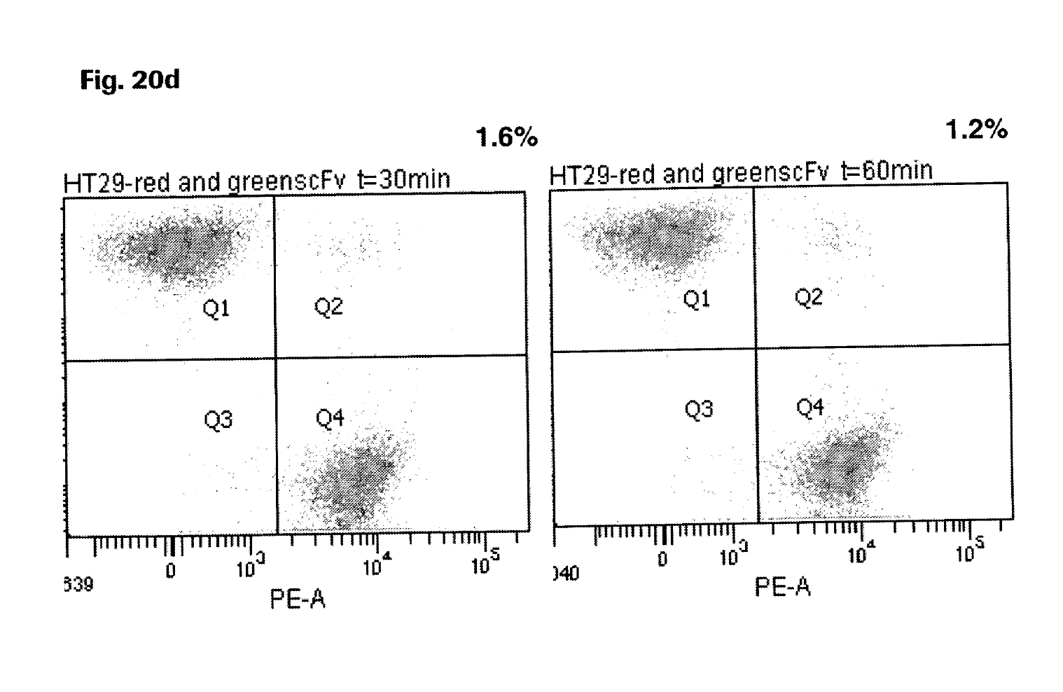

### FIG. 20

### Analysis of Cell-Cell Crosslinking by the Bispecific Her3/c-Met_scFv_SSKH Antibody in HT29 Cells

Due to the multivalency of the bispecific antibody format, cell-cell crosslinking is a possible mode of action which would also explain reduced receptor internalization. To study this phenomenon in more detail an experimental setup addressing this question was designed. For this purpose HT29 cells, expressing Her3 and c-Met on their cell surface, were split in two populations. One was stained with PKH26 (SIGMA), the other with PKH67 (Sigma), two membrane dyes the former green the latter red. Stained cells were mixed and incubated with Her3/c-Met_scFv_SSKH. In a flow cytometry based assay extensive crosslinking of cells would lead to an increase in the population of double positive (green+/red+) cells in the upper right quadrant. Based on this experiment no increase in cell-cell crosslinking could be observed under the given settings.

### Example 13

### Preparation of Glycoengineered of Bispecific Her3/c-Met Antibodies

The DNA sequences of bispecific Her3/c-Met antibody MH_TvAb18, MH_TvAb21 MH_TvAb 22, and MH_TvAb 30 were subcloned into mammalian expression vectors under the control of the MPSV promoter and upstream of a synthetic polyA site, each vector carrying an EBV OriP sequence.

Bispecific antibodies were produced by co-transfecting HEK293-EBNA cells with the mammalian bispecific antibody expression vectors using a calcium phosphate-transfection approach. Exponentially growing HEK293-EBNA cells were transfected by the calcium phosphate method. For the production of the glycoengineered antibody, the cells were co-transfected with two additional plasmids, one for a fusion GnTIII polypeptide expression (a GnT-III expression vector), and one for mannosidase II expression (a Golgi mannosidase II expression vector) at a ratio of 4:4:1:1, respectively. Cells were grown as adherent monolayer cultures in T flasks using DMEM culture medium supplemented with 10% FCS, and were transfected when they were between 50 and 80% confluent. For the transfection of a T150 flask, 15 million cells were seeded 24 hours before transfection in 25 ml DMEM culture medium supplemented with FCS (at 10% V/V final), and cells were placed at 37° C. in an incubator with a 5% CO2 atmosphere overnight. For each T150 flask to be transfected, a solution of DNA, CaCl2 and water was prepared by mixing 94 μg total plasmid vector DNA divided equally between the light and heavy chain expression vectors, water to a final volume of 469 μl and 469 μl of a 1M CaCl2 solution. To this solution, 938 μl of a 50 mM HEPES, 280 mM NaCl, 1.5 mM Na2HPO4 solution at pH 7.05 were added, mixed immediately for 10 sec and left to stand at room temperature for 20 sec. The suspension was diluted with 10 ml of DMEM supplemented with 2% FCS, and added to the T150 in place of the existing medium. Then additional 13 ml of transfection medium were added. The cells were incubated at 37° C., 5% CO2 for about 17 to 20 hours, then medium was replaced with 25 ml DMEM, 10% FCS. The conditioned culture medium was harvested 7 days post-transfection by centrifugation for 15 min at 210×g, the solution was sterile filtered (0.22 μm filter) and sodium azide in a final concentration of 0.01% w/v was added, and kept at 4° C.

The secreted bispecific afocusylated glycoengineered antibodies were purified by Protein A affinity chromatography, followed by cation exchange chromatography and a final size exclusion chromatographic step on a Superdex 200 column (Amersham Pharmacia) exchanging the buffer to 25 mM potassium phosphate, 125 mM sodium chloride, 100 mM glycine solution of pH 6.7 and collecting the pure monomeric IgG1 antibodies. Antibody concentration was estimated using a spectrophotometer from the absorbance at 280 nm.

The oligosaccharides attached to the Fc region of the antibodies were analyzed by MALDI/TOF-MS as described below (Example 14). Oligosaccharides were enzymatically released from the antibodies by PNGaseF digestion, with the antibodies being either immobilized on a PVDF membrane or in solution. The resulting digest solution containing the released oligosaccharides either prepared directly for MALDI/TOF-MS analysis or was further digested with EndoH glycosidase prior to sample preparation for MALDI/TOF-MS analysis.

### Example 14

### Analysis of Glycostructure of Bispecific Her3/c-Met Antibodies

For determination of the relative ratios of fucose- and non-fucose (a-fucose) containing oligosaccharide structures, released glycans of purified antibody material are analyzed by MALDI-T of-mass spectrometry. For this, the antibody sample (about 50 μg) is incubated over night at 37° C. with 5mU N-Glycosidase F (Prozyme# GKE-5010B) in 0.1M sodium phosphate buffer, pH 6.0, in order to release the oligosaccharide from the protein backbone. Subsequently, the glycan structures released are isolated and desalted using NuTip-Carbon pipet tips (obtained from Glygen: NuTip1-10 μl, Cat.Nr#NT1CAR). As a first step, the NuTip-Carbon pipet tips are prepared for binding of the oligosaccharides by washing them with 3 μL 1M NaOH followed by 20 μL pure water (e.g. HPLC-gradient grade from Baker, #4218), 3 μL 30% v/v acetic acid and again 20 μl pure water. For this, the respective solutions are loaded onto the top of the chromatography material in the NuTip-Carbon pipet tip and pressed through it. Afterwards, the glycan structures corresponding to 10 μg antibody are bound to the material in the NuTip-Carbon pipet tips by pulling up and down the N-Glycosidase F digest described above four to five times. The glycans bound to the material in the NuTip-Carbon pipet tip are washed with 20 μL pure water in the way as described above and are eluted stepwise with 0.5 μL 10% and 2.0 μL 20% acetonitrile, respectively. For this step, the elution solutions are filled in a 0.5 mL reaction vials and are pulled up and down four to five times each. For the analysis by MALDI-T of mass spectrometry, both eluates are combined. For this measurement, 0.4 μL of the combined eluates are mixed on the MALDI target with 1.6 μL SDHB matrix solution (2.5-Dihydroxybenzoic acid/2-Hydrorxy-5-Methoxybenzoic acid [Bruker Daltonics #209813] dissolved in 20% ethanol/5 mM NaCl at 5 mg/ml) and analyzed with a suitably tuned Bruker Ultraflex TOF/TOF instrument. Routinely, 50-300 shots are recorded and summed up to a single experiment. The spectra obtained are evaluated by the flex analysis software (Bruker Daltonics) and masses are determined for the each of the peaks detected. Subsequently, the peaks are assigned to fucose or a-fucose (non-fucose) containing glycol structures by comparing the masses calculated and the masses theoretically expected for the respective structures (e.g. complex, hybrid and oligo- or high-mannose, respectively, with and without fucose).

For determination of the ratio of hybride structures, the antibody sample are digested with N-Glycosidase F and Endo-Glycosidase H concomitantly N-glycosidase F releases all N-linked glycan structures (complex, hybride and oligo- and high mannose structures) from the protein backbone and the Endo-Glycosidase H cleaves all the hybride type glycans additionally between the two GlcNAc-residue at the reducing end of the glycan. This digest is subsequently treated and analyzed by MALDI-T of mass spectrometry in the same way as described above for the N-Glycosidase F digested sample. By comparing the pattern from the N-Glycosidase F digest and the combined N-glycosidase F/Endo H digest, the degree of reduction of the signals of a specific glyco structure is used to estimate the relative content of hybride structures.

The relative amount of each glycostructure is calculated from the ratio of the peak height of an individual glycol structure and the sum of the peak heights of all glyco structures detected. The amount of fucose is the percentage of fucose-containing structures related to all glyco structures identified in the N-Glycosidase F treated sample (e.g. complex, hybride and oligo- and high-mannose structures, resp.). The amount of afucosylation is the percentage of fucose-lacking structures related to all glyco structures identified in the N-Glycosidase F treated sample (e.g. complex, hybride and oligo- and high-mannose structures, resp.).

### Example 15

### In Vitro ADCC of Bispecific Her3/c-Met Antibodies

The Her3/c-Met bispecific antibodies according to the invention display reduced internalization on cells expressing both receptors. Reduced internalization strongly supports the rationale for glycoengineering these antibodies as a prolonged exposure of the antibody-receptor complex on the cell surface is more likely to be recognized by Nk cells. Reduced internalization and glycoengineering translate into enhanced antibody dependent cell cytotoxicity (ADCC) in comparison to the parent antibodies. An in vitro experimental setup to demonstrate these effects can be designed using cancer cells which express both Her3 and c-Met, on the cell surface, e.g. A431, and effector cells like a Nk cell line or PBMC's. Tumor cells are pre-incubated with the parent monospecific antibodies or the bispecific antibodies for up to 24 h followed by the addition of the effector cell line. Cell lysis is quantified and allows discrimination of mono- and bispecific antibodies.

The target cells, like A431 (cultivation in RPMI1640+2 mM L-Glutamine+10% FCS) (expressing both Her3 and c-Met) were collected with trypsin/EDTA (Gibco #25300-054) in exponential growth phase. After a washing step and checking cell number and viability the aliquot needed was labeled for 30 min at 37° C. in the cell incubator with calcein (Invitrogen #C3100MP; 1 vial was resuspended in 50 μl DMSO for 5 Mio cells in 5 ml medium). Afterwards, the cells were washed three times with AIM-V medium, the cell number and viability was checked and the cell number adjusted to 0.3 Mio/ml.

Meanwhile, PBMC as effector cells were prepared by density gradient centrifugation (Histopaque-1077, Sigma # H8889) according to the manufacturer's protocol (washing steps 1× at 400 g and 2× at 350 g 10 min each). The cell number and viability was checked and the cell number adjusted to 15 Mio/ml.

100 μl calcein-stained target cells were plated in round-bottom 96-well plates, 50 μl diluted antibody was added and 50 μl effector cells. In some experiments the target cells were mixed with Redimune® NF Liquid (ZLB Behring) at a concentration of 10 mg/ml Redimune.

As controls served the spontaneous lysis, determined by co-culturing target and effector cells without antibody and the maximal lysis, determined by 1% Triton X-100 lysis of target cells only. The plate was incubated for 4 hours at 37° C. in a humidified cell incubator.

The killing of target cells was assessed by measuring LDH release from damaged cells using the Cytotoxicity Detection kit (LDH Detection Kit, Roche #1 644 793) according to the manufacturer's instruction. Briefly, 100 μl supernatant from each well was mixed with 100 μl substrate from the kit in a transparent flat bottom 96 well plate. The Vmax values of the substrate's colour reaction was determined in an ELISA reader at 490 nm for at least 10 min. Percentage of specific antibody-mediated killing was calculated as follows: ((A−SR)/(MR−SR)×100, where A is the mean of Vmax at a specific antibody concentration, SR is the mean of Vmax of the spontaneous release and MR is the mean of Vmax of the maximal release.

### Example 16

### In Vivo Efficacy of Bispecific Her3/c-Met Antibodies in a Subcutaneous Xenograft Model with an Autocrine HGF Loop

A subcutaneous U87MG glioblastoma model has an autocrine HGF loop and displays Her3 and c-Met on the cell surface. Both receptors are phosphorylated in tumor explants which were lysed and subjected to immunoblot analysis (data not shown). U87MG cells are maintained under standard cell culture conditions in the logarithmic growth phase. Ten million cells are engrafted to SCID beige mice. Treatment starts after tumors are established and have reached a size of 100-150 mm3. Mice are treated with a loading dose of 20 mg/kg of antibody/mouse and then once weekly with 10 mg/kg of antibody/mouse. Tumor volume is measured twice a week and animal weights are monitored in parallel. Single treatments and combination of the single antibodies are compared to the therapy with bispecific antibody.

### Example 17

### In Vivo Efficacy of Bispecific HER3/c-Met Antibodies in a Subcutaneous Xenograft Model with a Paracrine HGF Loop

A subcutaneous BxPc-3 model, coinjected with Mrc-5 cells, mimics a paracrine activation loop for c-Met. BxPc-3 express c-Met as well as Her3 on the cell surface. BxPc-3 and Mrc-5 cells are maintained under standard cell culture conditions in the logarithmic growth phase. BxPc-3 and Mrc-5 cells are injected in a 10:1 ratio with ten million BxPc-3 cells and one million Mrc-5. Cells are engrafted to SCID beige mice. Treatment starts after tumors are established and have reached a size of 100-150 mm3. Mice are treated with a loading dose of 20 mg/kg of antibody/mouse and then once weekly with 10 mg/kg of antibody/mouse. Tumor volume is measured twice a week and animal weights are monitored in parallel. Single treatments and combination of the single antibodies are compared to the therapy with bispecific antibody.

### Example 18

### In Vivo Efficacy of Bispecific Her3/c-Met Antibodies in a Subcutaneous Xenograft Model with a Paracrine HGF Loop

Immunocompromised mice transgenic for human HGF serve as a source for systemic HGF. Such mice have been described in the literature and can be obtained from the Van Andel Institute. Subcutaneous injection of cancer cell lines, such as BxPc-3 or A549, expressing both receptors on the cell surface can be used to study efficacy of bispecific antibodies targeting Her3 and c-Met. Cells are maintained under standard cell culture conditions in the logarithmic growth phase. Ten million cells are engrafted to SCID beige mice carrying the transgene for HGF. Treatment starts after tumors are established and have reached a size of 100-150 mm3. Mice are treated with a loading dose of 20 mg/kg of antibody/mouse and then once weekly with 10 mg/kg of antibody/mouse. Tumor volume is measured twice a week and animal weights are monitored in parallel. Single treatments and combination of the single antibodies are compared to the therapy with bispecific antibody.

### Example 19

### In Vivo Efficacy of Bispecific Her3/c-Met Antibodies in an Orthotopic Xenograft Model with a Paracrine HGF Loop

A549 cancer cells express Her3 as well as c-Met on the cell surface. A549 cells are maintained under standard cell culture conditions in the logarithmic growth phase. Ten million cells are engrafted to SCID beige mice. Treatment starts after tumors are established and have reached a size of 100-150 mm3. Mice are treated with a loading dose of 20 mg/kg of antibody/mouse and then once weekly with 10 mg/kg of antibody/mouse. Tumor volume is measured twice a week and animal weights are monitored in parallel. Single treatments and combination of the single antibodies are compared to the therapy with bispecific antibody.

What is claimed is:

1. A bispecific antibody that specifically binds to human ErbB-3 and human c-Met comprising a first antigen-binding site that specifically binds to human ErbB-3 and a second antigen-binding site that specifically binds to human c-Met, wherein the bispecific antibody causes increase in internalization of ErbB-3 on A431 cells of no more than 15% when measured after 2 hours of A431 cell-antibody incubation as measured by a flow cytometry assay, as compared to internalization of ErbB-3 on A431 cells in the absence of antibody.

2. The antibody according to claim 1, wherein the antibody is a bivalent or trivalent bispecific antibody that specifically binds to human ErbB-3 and human c-Met, wherein the antibody comprises one or two antigen-binding sites that specifically bind to human ErbB-3 and one antigen-binding site that specifically binds to human c-Met.

3. The antibody according to claim 1 wherein the antibody is a trivalent, bispecific antibody that specifically binds to human ErbB-3 and human c-Met, wherein the antibody comprises two antigen-binding sites that specifically bind to human ErbB-3 and a third antigen-binding site that specifically binds to human c-Met.

4. The antibody according to claim 1 wherein the antibody is a bivalent, bispecific antibody that specifically binds to human ErbB-3 and human c-Met, wherein the antibody comprises one antigen-binding site that specifically binds to human ErbB-3 and a second antigen-binding site that specifically binds to human c-Met.

5. A bispecific antibody that specifically binds to human ErbB-3 and human c-Met, wherein the antibody comprises a first antigen-binding site that specifically binds to human ErbB-3 and a second antigen-binding site that specifically binds to human c-Met, wherein
i) the first antigen-binding site comprises in the heavy chain variable domain a CDR3H region with the amino acid sequence of SEQ ID NO: 53, a CDR2H region with the amino acid sequence of SEQ ID NO: 54, and a CDR1H region with the amino acid sequence of SEQ ID NO:55, and in the light chain variable domain a CDR3L region with the amino acid sequence of SEQ ID NO: 56, a CDR2L region with the amino acid sequence of SEQ ID NO:57, and a CDR1L region with the amino acid sequence of SEQ ID NO:58 or a CDR1L region with the amino acid sequence of SEQ ID NO:59; and
the second antigen-binding site comprises in the heavy chain variable domain a CDR3H region with the amino acid sequence of SEQ ID NO: 66, a CDR2H region with the amino acid sequence of SEQ ID NO: 67, and a CDR1H region with the amino acid sequence of SEQ ID NO: 68, and in the light chain variable domain a CDR3L region with the amino acid sequence of SEQ ID NO: 69, a CDR2L region with the amino acid sequence of SEQ ID NO: 70, and a CDR1L region with the amino acid sequence of SEQ ID NO: 71.
ii) the first antigen-binding site comprises in the heavy chain variable domain a CDR3H region with the amino acid sequence of SEQ ID NO: 60, a CDR2H region with the amino acid sequence of SEQ ID NO: 61, and a CDR1H region with the amino acid sequence of SEQ ID NO:62, and in the light chain variable domain a CDR3L region with the amino acid sequence of SEQ ID NO: 63, a CDR2L region with the amino acid sequence of SEQ ID NO:64, and a CDR1L region with the amino acid sequence of SEQ ID NO:65 or a CDR1L region with the amino acid sequence of SEQ ID NO:66; and
the second antigen-binding site comprises in the heavy chain variable domain a CDR3H region with the amino acid sequence of SEQ ID NO: 66, a CDR2H region with the amino acid sequence of, SEQ ID NO: 67, and a CDR1H region with the amino acid sequence of SEQ ID NO: 68, and in the light chain variable domain a CDR3L region with the amino acid sequence of SEQ ID NO: 69, a CDR2L region with the amino acid sequence of SEQ ID NO: 70, and a CDR1L region with the amino acid sequence of SEQ ID NO: 71.

6. The bispecific antibody according to claim 5, selected from the group of bispecific antibodies wherein
i) the first antigen-binding site comprises as heavy chain variable domain the amino acid sequence of SEQ ID NO: 47, and as light chain variable domain the amino acid sequence of SEQ ID NO: 48, and the second antigen-binding site comprises as heavy chain variable domain the amino acid sequence of SEQ ID NO: 3, and as light chain variable domain the amino acid sequence of SEQ ID NO: 4;
ii) the first antigen-binding site comprises as heavy chain variable domain the amino acid sequence of SEQ ID NO: 49, and as light chain variable domain the amino acid sequence of SEQ ID NO: 50, and the second antigen-binding site comprises as heavy chain variable domain the amino acid sequence of SEQ ID NO: 3, and as light chain variable domain the amino acid sequence of SEQ ID NO: 4;
iii) the first antigen-binding site comprises as heavy chain variable domain the amino acid sequence of SEQ ID NO: 49, and as light chain variable domain the amino acid sequence of SEQ ID NO: 51, and the second antigen-binding site comprises as heavy chain variable domain the amino acid sequence of SEQ ID NO: 3, and as light chain variable domain the amino acid sequence of SEQ ID NO: 4;
iv) the first antigen-binding site comprises as heavy chain variable domain the amino acid sequence of SEQ ID NO: 49, and as light chain variable domain the amino acid sequence of SEQ ID NO: 52, and the second antigen-binding site comprises as heavy chain variable domain the amino acid sequence of SEQ ID NO: 3, and as light chain variable domain the amino acid sequence of SEQ ID NO: 4; or
v) the first antigen-binding site comprises as heavy chain variable domain the amino acid sequence of SEQ ID NO: 1, and as light chain variable domain the amino acid sequence of SEQ ID NO: 2, and the second antigen-binding site comprises as heavy chain variable domain the amino acid sequence of SEQ ID NO: 3, and as light chain variable domain the amino acid sequence of SEQ ID NO: 4; or

7. The bispecific antibody according to claim 5, wherein
the first antigen-binding site comprises as heavy chain variable domain the amino acid sequence of SEQ ID NO: 49, and as light chain variable domain the amino acid sequence of SEQ ID NO: 51, and the second antigen-binding site comprises as heavy chain variable domain the amino acid sequence of SEQ ID NO: 3, and as light chain variable domain the amino acid sequence of a SEQ ID NO: 4;

8. The bispecific antibody according to claim 1, wherein the antibody comprises a constant region with the amino acid sequence of IgG1 or IgG3 subclass.

9. The bispecific antibody according to claim 1, wherein the antibody is glycosylated with a sugar chain at Asn297 and wherein the amount of fucose within the sugar chain is 65% or lower.

10. A nucleic acid encoding a bispecific antibody according to claim 1.

11. A nucleic acid encoding a bispecific antibody according to claim 5.

12. A nucleic acid encoding a bispecific antibody according to claim 6,

13. A pharmaceutical composition comprising a bispecific antibody according to claim 1.

14. A pharmaceutical composition comprising a bispecific antibody according to claim 5.

15. A pharmaceutical composition comprising a bispecific antibody according to claim 6.


SEQUENCE LISTING (93 sequences)

SEQ ID NO: 1 - protein, 118 aa, Mus musculus
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSS

SEQ ID NO: 2 - protein, 107 aa, Mus musculus
DIQMTQTTSSLSASLGDRVTISCRARQDISNYLNWYQRKPDGTVKLLIYYTSRLHSGVPSRFSGSGSGTDYSLTISNLEQEDIATYFCQQGNTFPWTFGGGTKLEIK

SEQ ID NO: 3 - protein, 119 aa, Artificial Sequence [sequence omitted]

SEQ ID NO: 4 - protein, 113 aa, Artificial Sequence [sequence omitted]

SEQ ID NO: 5 - protein, 448 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Description of Artificial Sequence Synthetic polypeptide
DIQMTQTTSSLSASLGDRVTISCRARQDISNYLNWYQRKPDGTVKLLIYYTSRLHSGVPSRFSGSGSGTDYSLTISNLEQEDIATYFCQQGNTFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 449 aa, Artificial Sequence
  Description of Artificial Sequence Synthetic polypeptide
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 220 aa, Artificial Sequence
  Description of Artificial Sequence Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 226 aa, Artificial Sequence
  Description of Artificial Sequence Synthetic polypeptide
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 10 - protein, 220 aa, Artificial Sequence; identical to SEQ ID NO: 8
  Description of Artificial Sequence Synthetic polypeptide

SEQ ID NO: 11 - protein, 582 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSS

SEQ ID NO: 12 - protein, 577 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 13 - protein, 214 aa, Artificial Sequence; identical to SEQ ID NO: 6
  Description of Artificial Sequence Synthetic polypeptide

SEQ ID NO: 14 - protein, 582 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSS

SEQ ID NO: 15 - protein, 577 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGCGTKVEIKR

SEQ ID NO: 16 - protein, 214 aa, Artificial Sequence; identical to SEQ ID NO: 6
  Description of Artificial Sequence Synthetic polypeptide

SEQ ID NO: 17 - protein, 582 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSS

SEQ ID NO: 18 - protein, 577 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGCGTKVEIKR

SEQ ID NO: 19 - protein, 214 aa, Artificial Sequence; identical to SEQ ID NO: 6
  Description of Artificial Sequence Synthetic polypeptide

SEQ ID NO: 20 - protein, 572 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSS

SEQ ID NO: 21 - protein, 567 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 22 - protein, 214 aa, Artificial Sequence; identical to SEQ ID NO: 6
  Description of Artificial Sequence Synthetic polypeptide

SEQ ID NO: 23 - protein, 597 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSS

SEQ ID NO: 24 - protein, 592 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 25 - protein, 214 aa, Artificial Sequence; identical to SEQ ID NO: 6
  Description of Artificial Sequence Synthetic polypeptide

SEQ ID NO: 26 - protein, 706 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGCGTKVEIKR

SEQ ID NO: 27 - protein, 448 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 214 aa, Artificial Sequence; identical to SEQ ID NO: 6
  Description of Artificial Sequence Synthetic polypeptide

SEQ ID NO: 29 - protein, 706 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGCGTKVEIKR

SEQ ID NO: 30 - protein, 448 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 214 aa, Artificial Sequence; identical to SEQ ID NO: 6
  Description of Artificial Sequence Synthetic polypeptide

SEQ ID NO: 32 - protein, 706 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 33 - protein, 448 aa, Artificial Sequence; identical to SEQ ID NO: 30
  Description of Artificial Sequence Synthetic polypeptide

SEQ ID NO: 34 - protein, 214 aa, Artificial Sequence; identical to SEQ ID NO: 6
  Description of Artificial Sequence Synthetic polypeptide

SEQ ID NO: 35 - protein, 706 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVEGFYPSDIAVEWESNGQPENNYKTTPPVLDSDGSFFLYSDLTVDKSRWQQGNVFSCSVMHEALHNHYTQKSLSLSPGKGGGGSGGGGSEVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGCGTKVEIKR

SEQ ID NO: 36 - protein, 448 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDKLTKNQVSLSCAVKGFYPSDIAVEWESNGQPENNYKTTPPVLKSDGSFFLVSKLTVDKSRWQQGNVFSCSVMHEALHNHYTQKSLSLSPGK

SEQ ID NO: 37 - protein, 214 aa, Artificial Sequence; identical to SEQ ID NO: 6
  Description of Artificial Sequence Synthetic polypeptide

SEQ ID NO: 38 - protein, 706 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLWCLVEGFYPSDIAVEWESNGQPENNYKTTPPVLDSDGSFFLYSDLTVDKSRWQQGNVFSCSVMHEALHNHYTQKSLSLSPGKGGGGSGGGGSEVQLVESGGGLVQPGGSLRLSCAASGYTFTSYWLHWVRQAPGKCLEWVGMIDPSNSDTRFNPNFKDRFTISADTSKNTAYLQMNSLRAEDTAVYYCATYRSYVTPLDYWGQGTLVTVSSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGCGTKVEIKR

SEQ ID NO: 39 - protein, 448 aa, Artificial Sequence
  Description of Artificial Sequence Synthetic polypeptide
DVQLQESGPGLVKPSQSLSLTCSVTGYSITSAYYWNWIRQFPGNKLEWMGYISYGGSNSYAPSLKNRFSITRDTSKNQFFLKLNSVTTEDTATYYCARESDYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDKLTKNQVSLSCAVKGFYPSDIAVEWESNGQPENNYKTTPPVLKSDGSFFLVSKLTVDKSRWQQGNVFSCSVMHEALHNHYTQKSLSLSPGK

SEQ ID NO: 40 - protein, 214 aa, Artificial Sequence; identical to SEQ ID NO: 6
  Description of Artificial Sequence Synthetic polypeptide

SEQ ID NO: 41 - protein, 330 aa, Homo sapiens [sequence omitted]

SEQ ID NO: 42 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 43 - protein, 107 aa, Homo sapiens [sequence omitted]

SEQ ID NO: 44 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 45 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 46 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 47 - protein, 120 aa, Mus musculus
QGQMQQSGAELVKPGASVKLSCKTSGFTFRSSYISWLKQKPRQSLEWIAWIYAGTGSPSYNQKFTGKAQLTVDTSSSTAYMQLSSLTSEDSAIYYCARHRDYYSNSLTYWGQGTLVTVSA

SEQ ID NO: 48 - protein, 113 aa, Mus musculus
DIVMTQSPSSLTVTAGENVTMSCKSSQSLLNSGNQKNYLTWYQQKPGQPPKLLIYWASTRESGVPNRFTGSGSGTDFTLAISSVQAEDLSIYYCQSDYSYPYTFGQGTKLEIK

SEQ ID NO: 49 - protein, 120 aa, Artificial Sequence
  Description of Artificial Sequence Synthetic polypeptide
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSS

SEQ ID NO: 50 - protein, 113 aa, Artificial Sequence
  Description of Artificial Sequence Synthetic polypeptide
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQSDYSYPYTFGQGTKLEIK

SEQ ID NO: 51 - protein, 113 aa, Artificial Sequence
  Description of Artificial Sequence Synthetic polypeptide
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIK

SEQ ID NO: 52 - protein, 113 aa, Artificial Sequence
  Description of Artificial Sequence Synthetic polypeptide
DIVMTQSPDSLAVSLGERATINCKSSQSLLNSGNQKNYLTWYQQKPGQPPKLLIYWASTRESGVPDRFSGSGSGTDFTLTISSLQAEDVSIYYCQSDYSYPYTFGQGTKLEIK

SEQ ID NO: 53 - protein, 11 aa, Artificial Sequence
  Description of Artificial Sequence Synthetic peptide
HRDYYSNSLTY

SEQ ID NO: 54 - protein, 17 aa, Artificial Sequence
  Description of Artificial Sequence Synthetic peptide
WISAYNGNTNYAQKLQG

SEQ ID NO: 55 - protein, 10 aa, Artificial Sequence
  Description of Artificial Sequence Synthetic peptide
GYTFRSSYIS

SEQ ID NO: 56 - protein, 9 aa, Artificial Sequence
  Description of Artificial Sequence Synthetic peptide
QSDYSYPYT

SEQ ID NO: 57 - protein, 7 aa, Artificial Sequence
  Description of Artificial Sequence Synthetic peptide
WASTRES

SEQ ID NO: 58 - protein, 17 aa, Artificial Sequence
  Description of Artificial Sequence Synthetic peptide
KSSQSLLNSGNQKNYLT

SEQ ID NO: 59 - protein, 17 aa, Artificial Sequence
  Description of Artificial Sequence Synthetic peptide
KSSQSVLNSGNQKNYLT

SEQ ID NO: 60 - protein, 9 aa, Artificial Sequence
  Description of Artificial Sequence Synthetic peptide
ESDYAYFDY

SEQ ID NO: 61 - protein, 16 aa, Mus musculus
YISYGGSNSYAPSLKN

SEQ ID NO: 62 - protein, 6 aa, Mus musculus
SAYYWN

SEQ ID NO: 63 - protein, 9 aa, Mus musculus
QQGNTFPWT

SEQ ID NO: 64 - protein, 7 aa, Mus musculus
YTSRLHS

SEQ ID NO: 65 - protein, 11 aa, Mus musculus
RARQDISNYLN

SEQ ID NO: 66 - protein, 10 aa, Artificial Sequence
  Description of Artificial Sequence Synthetic peptide
YRSYVTPLDY

SEQ ID NO: 67 - protein, 17 aa, Artificial Sequence
  Description of Artificial Sequence Synthetic peptide
MIDPSNSDTRFNPNFKD

SEQ ID NO: 68 - protein, 5 aa, Artificial Sequence
  Description of Artificial Sequence Synthetic peptide
SYWLH

SEQ ID NO: 69 - protein, 9 aa, Artificial Sequence
  Description of Artificial Sequence Synthetic peptide
QQYYAYPWT

SEQ ID NO: 70 - protein, 7 aa, Artificial Sequence
  Description of Artificial Sequence Synthetic peptide
WASTRES

SEQ ID NO: 71 - protein, 17 aa, Artificial Sequence
  Description of Artificial Sequence Synthetic peptide
KSSQSLLYTSSQKNYLA

SEQ ID NO: 72 - protein, 24 aa, Artificial Sequence
  Description of Artificial Sequence Synthetic peptide
GGGSGGGSGGGSGGGSGGGSGGGS

SEQ ID NO: 73 - protein, 27 aa, Artificial Sequence
  Description of Artificial Sequence Synthetic peptide
GGGSGGGSGGGSGGGSGGGSGGGSGGG

SEQ ID NO: 74 - protein, 25 aa, Artificial Sequence
  Description of Artificial Sequence Synthetic peptide
GGGGSGGGGSGGGGSGGGGSGGGGS

SEQ ID NO: 75 - protein, 28 aa, Artificial Sequence
  Description of Artificial Sequence Synthetic peptide
GGGGSGGGGSGGGGSGGGGSGGGGSGGG

SEQ ID NO: 76 - protein, 32 aa, Artificial Sequence
  Description of Artificial Sequence Synthetic polypeptide
GGGSGGGSGGGSGGGSGGGSGGGSGGGSGGGS

SEQ ID NO: 77 - protein, 35 aa, Artificial Sequence
  Description of Artificial Sequence Synthetic polypeptide
GGGSGGGSGGGSGGGSGGGSGGGSGGGSGGGSGGG

SEQ ID NO: 78 - protein, 35 aa, Artificial Sequence
  Description of Artificial Sequence Synthetic polypeptide
GGGGSGGGGSGGGGSGGGGSGGGGSGGGGSGGGGS

SEQ ID NO: 79 - protein, 38 aa, Artificial Sequence
  Description of Artificial Sequence Synthetic polypeptide
GGGGSGGGGSGGGGSGGGGSGGGGSGGGGSGGGGSGGG

SEQ ID NO: 80 - protein, 43 aa, Artificial Sequence
  Description of Artificial Sequence Synthetic polypeptide
GGGSGGGSGGGSGGGSGGGSGGGSGGGSGGGSGGGSGGGSGGG

SEQ ID NO: 81 - protein, 43 aa, Artificial Sequence
  Description of Artificial Sequence Synthetic polypeptide
GGGGSGGGGSGGGGSGGGGSGGGGSGGGGSGGGGSGGGGSGGG

SEQ ID NO: 82 - protein, 32 aa, Artificial Sequence
  Description of Artificial Sequence Synthetic polypeptide
GGGGSGGGGSGGGGSGGGGSGGGGSGGGGSGG

SEQ ID NO: 83 - protein, 24 aa, Artificial Sequence
  Description of Artificial Sequence Synthetic peptide
GGGSGGGSGGGSGGGSGGGSGGGS

SEQ ID NO: 84 - protein, 30 aa, Artificial Sequence
  Description of Artificial Sequence Synthetic polypeptide
GGGGSGGGGSGGGGSGGGGSGGGGSGGGGS

SEQ ID NO: 85 - protein, 15 aa, Artificial Sequence
  Description of Artificial Sequence Synthetic peptide
GGGGSGGGGSGGGGS

SEQ ID NO: 86 - protein, 20 aa, Artificial Sequence
  Description of Artificial Sequence Synthetic peptide
GGGGSGGGGSGGGGSGGGGS

SEQ ID NO: 87 - protein, 5 aa, Artificial Sequence
  Description of Artificial Sequence Synthetic peptide
GGGGS

SEQ ID NO: 88 - protein, 19 aa, Artificial Sequence
  Description of Artificial Sequence Synthetic peptide
MGWSCIILFLVATATGVHS

SEQ ID NO: 89 - protein, 10 aa, Artificial Sequence
  Description of Artificial Sequence Synthetic peptide
GGGGSGGGGS

SEQ ID NO: 90 - protein, 16 aa, Artificial Sequence
  Description of Artificial Sequence Synthetic peptide
GGGSGGGSGGGSGGGS

SEQ ID NO: 91 - protein, 28 aa, Artificial Sequence
  Description of Artificial Sequence Synthetic peptide
GGGSGGGSGGGSGGGSGGGSGGGSGGGS

SEQ ID NO: 92 - protein, 27 aa, Artificial Sequence
  Description of Artificial Sequence Synthetic peptide
GGGGSGGGGSGGGGSGGGGSGGGGSGG

SEQ ID NO: 93 - protein, 30 aa, Artificial Sequence; identical to SEQ ID NO: 84
  Description of Artificial Sequence Synthetic polypeptide